(19) United States
(12) Patent Application Publication
(10) Pub. No.: US 2005/0203014 A1
(43) Pub. Date: Sep. 15, 2005

(54) Composition and methods for modulating CNS activity

(75) Inventor: Lee L. Rubin, Wellesley, MA (US)

Correspondence Address: FISH & NEAVE IP GROUP;ROPES & GRAY LLP, ONE INTERNATIONAL PLACE, BOSTON, MA, 02110-2624, US

(73) Assignee: Curis, Inc., Cambridge, MA (US)

(21) Appl. No.: 11/018,739

(22) Filed: Dec. 20, 2004

Related U.S. Application Data
(63) Continuation-in-part of application No. PCT/US04/42271, filed on Dec. 15, 2004.
(60) Provisional application No. 60/531,201, filed on Dec. 19, 2003.

(51) Int. Cl. A61K 38/17; A61K 48/00; C12Q 1/68

(52) U.S. Cl. 514/12; 435/6; 514/44

(57) ABSTRACT

The present invention concerns the methods and compositions for treating depression and other behavioral and/or emotional disorders of the central nervous system by administering an agonist of hedgehog signaling. Other disorders amenable to treatment by the subject method include attention deficit hyperactive disorders, non-Alzheimer dementia, and various symptoms of memory loss. The present invention also concerns the methods and compositions for enhancing memory and/or cognitive functions, both in a patient suffering from ailment affecting these functions, and in a subject with no diagnosed deficit in memory or cognitive function. The methods and compositions of the present invention stimulate neurogenesis and differentiation, and enhance synaptic transmission of neurons.

### RELATED APPLICATIONS

This application is a continuation of International Application Serial No. [not yet assigned], filed Dec. 15, 2004, entitled COMPOSITION AND METHODS FOR MODULATING CNS ACTIVITY, which claims priority to U.S. Provisional Application No. 60/531,201, filed Dec. 19, 2003, the specifications of each of which are incorporated by reference herein.

### BACKGROUND OF THE INVENTION

The hh Gene Family

The first hedgehog (hh) gene was identified by a genetic screen in the fruitfly Drosophila melanogaster (Nüsslein-Volhard, C. and Wieschaus, E. (1980) Nature 287, 795-801). This screen identified a number of mutations affecting embryonic and larval development. In 1992 and 1993, the molecular nature of the Drosophila hh gene was reported (Cf, Lee et al. (1992) Cell 71, 33-50), and since then, several hh homologues have been isolated from various vertebrate species. While only one hh gene has been found in the genome of Drosophila and other invertebrates, multiple hh genes are present in vertebrates.

The vertebrate family of hh genes includes at least four members, i.e., paralogs, of the single Drosophila hh gene. Exemplary hh genes and proteins are described in PCT publications WO 95/18856 and WO 96/17924. Three of these members, herein referred to as desert hedgehog (Dhh), sonic hedgehog (Shh) and indian hedgehog (Ihh), apparently exist in all vertebrates, including fish, birds, and mammals. A fourth member, herein referred to as tiggie-winkle hedgehog (Thh), appears specific to fish. Dhh is expressed principally in the testes, both in mouse embryonic development and in the adult rodent and human; Ihh is involved in bone development during embryogenesis and in bone formation in the adult; and, Shh, as described above, is primarily involved in morphogenic and neuroinductive activities.

The various Hedgehog (Hh) proteins consist of a signal peptide, a highly conserved N-terminal region, and a more divergent C-terminal domain. In addition to signal sequence cleavage in the secretory pathway, Hh precursor proteins undergo an internal autoproteolytic cleavage which depends on conserved sequences in the C-terminal portion. This autocleavage leads to a 19 kDa N-terminal peptide, which stays tightly associated with the surface of cells in which it was synthesized, and a C-terminal peptide of 26-28 kDa, which is freely diffusible both in vitro and in vivo. Biochemical studies have shown that the autoproteolytic cleavage of the Hh precursor protein proceeds through an internal thioester intermediate which subsequently is cleaved in a nucleophilic substitution. It is likely that the nucleophile is a small lipophilic molecule which becomes covalently bound to the C-terminal end of the N-peptide, tethering it to the cell surface. As a result of the tethering, a high local concentration of N-terminal Hh peptide is generated on the surface of the Hh producing cells. It is this N-terminal peptide which is both necessary and sufficient for short- and long-range Hh signaling activities in Drosophila and vertebrates.

Role of Hedgehog in Development and Differentiation

Members of the Hh family of signaling molecules mediate many important short- and long-range patterning processes during invertebrate and vertebrate development. In the fly, a single hh gene regulates segmental and imaginal disc patterning. In the establishment of segment polarity in early embryos, it has short-range effects which appear to be directly mediated, while in the patterning of the imaginal discs, it induces long-range effects via the induction of secondary signals. In contrast, in vertebrates, the hh gene family is involved in the control of left-right asymmetry, polarity in the CNS, somites and limb, organogenesis, chondrogenesis and spermatogenesis.

In vertebrates, several hh genes have been cloned in the past few years. Of these genes, Shh has received most of the experimental attention, as it is expressed in different organizing centers which are the sources of signals that pattern neighboring tissues. Recent evidence indicates that Shh is involved in these interactions, which were observed in the laboratory in mouse, rat, chick, and zebrafish. Shh appears to play an important role in the development of the central nervous system (“CNS”).

Detailed description of the Hh family of proteins and their roles in development can be found in U.S. Publication No. 2003-0139457, the disclosure of which is incorporated herein by reference in its entirety.

One protein known to directly interact with Hh polypeptide is encoded by a gene patched (Chen, Y. et al. (1996) Cell 87:553). Patched was originally identified in Drosophila as a segment polarity gene, one of a group of developmental genes that affect cell differentiation. Patched proteins possess two large extracellular domains, twelve transmembrane segments, and several cytoplasmic segments. See Hooper, J. E. et al. (1989) Cell 59:751; and Nakano, Y. et al. (1989) Nature 341:508; Johnson, R. L. et al. (1996) Science 272:1668; and Hahn, H. et al. (1996) Cell 85:841. Genetic and functional studies demonstrate that patched is part of the Hh signaling cascade, an evolutionarily conserved pathway that regulates expression of a number of downstream genes. See Perrimon, N. (1995) Cell 80:517; and Perrimon, N. (1996) Cell 86:513. Patched is thought to participate in a Hh receptor complex along with another transmembrane protein encoded by the smoothened gene.

The human homologue of patched was cloned and mapped to chromosome 9q22.3. See Johnson, supra; and Hahn, supra.

The smoothened gene encodes a transmembrane protein, which is downstream of the receptor and through which the Hh signal is transmitted into an intracellular signal. Alcedo, J. et al. (1996) Cell 86(2): 221-232; van den Heuvel, M. et al. (1996) Nature 382:547-551. Also found in the Hh signaling pathway are proteins encoded by gli-1, gli-2 and gli-3 genes. Gli-1 is an activating transcription factor and Gli-3 is a repressive transcription factor. Dai, P. et al. (1999) J. Biol. Chem. 274:8143-8152.

Depression

According to the National Institute of Mental Health, each year, about 19 million American adults suffer from some form of depression, equaling to approximately 1 in 10 adults. Women are twice as likely to experience depressive episodes as men. However, depression in men is thought to be underreported and often obscured behind a variety of physical complaints, such as low energy, aches and pains, a loss of appetite, or trouble sleeping.

Depression strikes as many as 2.5% of children and 8.3% of teens in the United States. While a full-blown depression most often starts in mid-adulthood, low-grade depression, or dysthymia, may begin during childhood or the teenage years. Depression in children and teens often coexists with behavioral problems, anxiety, or substance abuse. According to a study in the American Journal of Psychiatry, age alone does not seem to have any significant effect on depression. However, depression is likely underreported in older people. The symptoms of depression are sometimes mistaken for dementia, and the stigmatic view of depression as a personal weakness is still widespread among the older generation. Lack of Medicare coverage for the treatment of depression may also result in underreporting.

Three main categories of depression are currently known: major depression, or unipolar depression; dysthymia, a lasting, low-level depression; and bipolar disorder, also known as bipolar affective disorder or manic depression. Another category is termed cyclothymia, which is marked by manic and depressive states, yet neither are of sufficient intensity nor duration to merit a diagnosis of bipolar disorder or major depressive disorder.

Major depression lasts at least two weeks, during which time a patient experiences at least four of the following signs of depression: a change in appetite that sometimes leads to weight loss or gain; insomnia or, less often, oversleeping; a slowdown in talking and performing other tasks or, conversely, restlessness and an inability to sit still; loss of energy or feeling tired much of the time; feelings of worthlessness or excessive, inappropriate guilt.

Dysthymia is a low-level drone of depression that lasts for at least two years in adults and one year in children and teens. The depressed mood does not lift for more than two months, and at least two of the following symptoms are seen: overeating or a loss of appetite; insomnia or sleeping too much; little energy or feeling tired; low self-esteem; trouble concentrating or making decisions; hopelessness.

Bipolar disorder always includes one or more episodes of high or manic behavior. It also often includes episodes of depression. During a manic episode, a patient typically feels terrifically elated, expansive, or irritated over the course of a week or longer, accompanied with at least three of the following symptoms: grandiose ideas or pumped-up self-esteem; far less need for sleep than normal; an urgent desire to talk; racing thoughts and distractibility; increased activity that may be poured into a goal or expressed as agitation; a pleasure-seeking urge that might be funneled into sexual sprees, over-spending, or a variety of schemes, often with disastrous consequences.

Depression has many potential causes. Often it is triggered not by a single factor but by a combination of several factors, e.g., genetic vulnerability, certain forms of stress, or change in brain chemistry. The link between stress and depression has been suggested, and certain kinds of stress have greater impact, e.g., early-life trauma or losses (such as physical or sexual abuse in childhood, death of a parent or the withdrawal of a loved one's affection). Studies indicate that the experience of these events increases the risk of developing depression later in life. Depression can also be recurring. Studies show that major depression is a highly recurrent illness. See, e.g. Solomon, D. A. et al. (2001) Am. J. Psychiatry 158(5):819-20; Stoudemire, A. (1997) J Neuropsychiatry Clin. Neurosci. 9(2):208-21. Other forms of depression such as bipolar disorder are also considered to be highly recurring.

Genetic studies show that while no single gene prompts depression, a combination of genetic variation may heighten your vulnerability to certain forms of this disease. The genetic components of depression have not been pinned down. The clearest genetic connection is seen in bipolar disorder (e.g., a first degree relative's experience of full-blown mania results in a 12% change of developing bipolar disorder). Experts believe that depressive disorders probably result when genetic variations that create vulnerability interact with and are amplified by environmental factors, including early-life trauma or losses or chronic stress.

Symptoms of depression or mania can also be a side effect of certain medications, such as steroids or blood pressure medication. Medical illnesses or medications are thought to be at the root of about 10%-15% of all depressions. Among the best known medical causes of mood disorder are two thyroid hormone imbalances. An excess of thyroid hormone, or hyperthyroidism, can trigger manic symptoms. Hypothyroidism, a condition in which too little thyroid hormone is produced in the body, often leads to exhaustion and depression, and affects millions of Americans, mainly women or the elderly. Other possible medical causes of mood disorder include: degenerative neurological conditions, such as multiple sclerosis, Parkinson's disease, Alzheimer's disease, and Huntington's disease; stroke; certain nutritional deficiencies, such as a lack of vitamin B12; other endocrine disorders, such as over- or under-activity of the parathyroid or adrenal glands resulting in hormonal imbalance; certain immune disorders, such as lupus; certain infectious diseases, such as mononucleosis, hepatitis, and human immunodeficiency virus (HIV); certain cancers, such as pancreatic or brain cancer.

However, the causal relationships between the onset of depression and these medical conditions, with the associated stress, are unclear. Studies have indicated that elders who suffer from chronic depression lasting at least six years have an 88% higher risk of getting all types of cancer. Other research indicates that depression slows the recovery of seniors hospitalized for health reasons. A connection between Alzheimer's disease and depression has also been hinted: a higher risk of developing Alzheimer's disease or experiencing a decline in mental powers is noted among those who were depressed, although only among those with more than eight years of education.

Additionally, hormones may also play a role in depression. Estrogen and progesterone may play a role in depression for some women suffering from premenstrual syndrome (“PMS”). Postpartum depression, the weepy, anxious, emotional rollercoaster known as the “baby blues,” which 70% of new mothers experience within the first 10 days after childbirth, is another form of depressive disorders that affects women only. In men, decrease of testosterone levels as they age seems to be a link to depression, irritability, anxiety, low energy, poor concentration and memory, and disturbed sleep.

Studies have shown neurological and biochemical changes in the brain of a patient suffering from depression, which may explain the symptoms of depression such as derailed sleep, suppressed appetite, agitation, exhaustion, or apathy. The homeostasis of neurotransmitters in the brain necessary for the normal functioning of the brain may be disrupted or imbalanced in severely depressed or manic patients. For example, receptors may be oversensitive or insensitive to a specific neurotransmitter, resulting in excessive or inadequate response to a given amount of the neurotransmitter. Conversely, the amount of a neurotransmitter available to its receptor may be too much or too little, caused by abnormal levels of its production and secretion, or its reuptake or exclusion from the system. Therefore, restoring the balance of such neurotransmitter signaling is an effective treatment for depression in some cases. For example, serotonin, a neurotransmitter that helps regulate sleep, appetite, moods, and inhibits pain, is found to be at low levels in depressed people; selective serotonin reuptake inhibitors (SSRIs), e.g., floxetine, increase the available concentration of serotonin by limiting its uptake, and is effective as antidepressants for some depressed patients.

In addition to biochemical changes, depression is associated with physical changes in the brain of a patient, which may include lesions, loss of neurons or atrophy of certain regions of the brain such as hippocampus and prefrontal cortex. See, for example, Sheline, Y. I. et al., (1996) Proc. Nat. Acad. Sci. USA 93: 3908-3913. Although it is unclear whether these cell losses contribute to the pathogenesis or are consequences of depression, there are indications that antidepressants may reverse such neurological alterations and help with the symptoms.

Typically, physicians diagnose depression by investigating possible medical causes and eliciting information that helps color in the picture. The first step may be a physical exam or screening tools such as self-report scales (a checklist of symptoms to fill out), scales completed by a clinician, and/or or a clinical interview by a doctor or therapist based on the key criteria for depression or bipolar disorder. Other tests can also be useful to confirm a diagnosis, tease out information, or distinguish depression from other psychological or neurological problems; they include psychological tests, such as the Minnesota Multi-phasic Personality Inventory (“MMPI”), the Rorschach (“inkblot”), or Thematic Apperception Test, neuropsychological tests such as the modified Halstead-Reitan battery, neurological tests such as an electroencephalogram (“EEG”) or MRI, and/or tests for biological causes of depression such as tests of thyroid function.

Attention Deficit Disorders

An attention-deficit disorder (ADD) is a developmental disorder characterized by developmentally inappropriate degrees of inattention, overactivity, and impulsivity. Symptoms are neurologically-based, arise in early childhood, and are chronic in nature in most cases. Symptoms are not due to gross neurological impairment, sensory impairment, language or motor impairment, mental retardation, or emotional disturbance.

ADD with and without hyperactivity are separate and unique childhood disorders. They are not subtypes of an identical attention disturbance. It has been noted that children with ADD/−H are more frequently described as depressed, learning disabled, or “lazy” while children with ADD/+H are more frequently labeled as conduct or behavior disordered.

Memory and Cognitive Functions

Memory, or the function of a living organism to store information and retrieve it at a later time in a functional form, comprises multiple processes and requires the function of many different brain areas. Human memory provides declarative recall, i.e., facts and events accessible to conscious recollection, and non-declarative recall, i.e., procedural memory of skills and operations not stored regarding time and place.

The processing of information to be added to memory occurs in several stages. A newly acquired experience initially is susceptible to various forms of disruption. With time, however, the new experience becomes resistant to disruption. This observation has been interpreted to indicate that a labile, working, short-term memory is “consolidated” into a more stable, long term memory. The initial phase of memory consolidation occurs in the first few minutes after we are exposed to a new idea or learning experience. The next phase occurs over a longer period of time, such as during sleep. If a learning experience has on-going meaning to us, the next week or so serves as a further period of memory consolidation. In effect, in this phase, the memory moves from short-term to long-term storage.

Various mechanisms have been proposed for the formation of long-term memory. A wide range of observations suggest an evolutionarily conserved molecular mechanism for the formation of long-term memory. These observations include increase in release of synaptic transmitter and number of synaptic receptors as well as decrease in Km of the receptors, synthesis of new memory factors either in the pre-synaptic or post-synaptic element, new synaptic connections, and increase in the active area in the pre-synaptic membrane. Synaptic plasticity, the change in the strength of neuronal connections in the brain, is thought to underlie long-term memory storage.

On the molecular level, a series of classic studies showed that inhibition of mRNA and protein synthesis during a critical time window could disrupt the formation of long-term memory. Initial learning and recall of previously stored information was not impaired by the transient blockage of protein synthesis. This led to a hypothesis that new gene expression is necessary for the conversion or consolidation of a short-term modification of the brain into a long-term memory.

Memory consolidation, or long-term memory, is also believed to play a crucial role in a variety of neurological and mental disorders, including mental retardation, Alzheimer's disease and depression. Indeed, loss or impairment of long-term memory is significant feature of such diseases.

Dementia

Dementia is defined as a mental disorder characterized by a decline of previously attained intellectual abilities, involving personality changes and impairment of memory, judgement and abstract thinking. It is more or less sustained in time, arbitrarily measurable in months or years rather than in days or weeks. Although long lasting, some varieties of dementia may be arrested or reversed. The term “dementia” is not applied to isolated focal loss of function such as occurs in amnesia, aphasia, agnosia, or apraxia. The decline usually involves memory, other cognitive capacities, and adaptive behavior. There is usually no major alteration of consciousness. The patient may or may not be aware of the dementia. In almost all cases, there is significant deterioration of memory and of one or more other intellectual functions such as language, spatial or temporal orientation, judgment, and abstract thought. Some criteria for dementia require defects in one or more components of intellectual function other than memory; some require that the defect be global, that is, involve all components of intellectual function.

Dementia can be caused by a number of brain disorders, including Alzheimer's disease, Huntington's disease, multiple sclerosis and Parkinson's disease. Other types of dementia are vascular, or multi-infarct dementia, Lewy body dementia, frontal lobe dementia such as Pick's disease, subcortical dementias (such as Huntington or progressive supranuclear palsy), focal cortical atrophy syndromes (such as primary aphasia), metabolic-toxic dementias (such as chronic hypothyroidism or B12 deficiency), and dementias caused by infections (such as syphilis, neuroAIDS or chronic meningitis).

### BRIEF SUMMARY OF THE INVENTION

The present invention provides methods for modulating activities of central nervous system (“CNS”) through modification of the signaling through the Hh family of proteins in the CNS. The present invention thus provides methods for treating behavioral and emotional disorders as well as for enhancing or restoring memory and/or cognitive function of a subject.

One aspect of the present invention provides methods for modulating activity of CNS of a mammal by stimulating neuronal stem cells via a Hh signaling pathway, thereby promoting differentiation and/or migration of the neuronal stem cells and/or directly regulating the synaptic transmission of the basal ganglia region. A Hh signaling pathway may be activated by a Hh polypeptide or any agonist of Hh activity that mimics its activity, or any compound or composition that ultimately increases activity of Gli, especially Gli-1. The Hh signaling pathway may, for example, be enhanced by an antagonist of the negative regulatory elements or negative feedback elements within the pathway (e.g., an antagonist of the patched receptor).

Moreover, the present invention provides methods for treating behavioral and/or emotional disorders by modulating the activity of CNS via the Hh signaling pathway.

Another aspect of the present invention is method of enhancement of cognitive function and memory function of a patient. Activating the Hh signaling pathway, thereby stimulating differentiation and migration of neuronal stem cells, by various agents results in improved cognition and memory. Yet another aspect of the present invention provides methods of treatment of disorders which are accompanied by neuronal cell loss or lesion, by stimulating neurogenesis, activating the neuronal stem cells to differentiate and migrate to the site of the damage. Such differentiation and migration can be promoted by activating the Hh signaling pathway by various agents. The enhanced neurogenesis may augment already-upregulated neurogenesis, which may be a body's remedial response to the pathological state.

An aspect of the present invention also provides enhancement of memory and cognition, caused by diseases such as AIDS-related dementia, and to alleviate symptoms of these diseases and other disorders such as depression which correlate with degradation of memory and cognitive functions. Additionally, the present invention provides enhancement of memory and cognition in subjects who do not suffer from general symptoms of memory- and cognition-impairing disorders, but still benefit from improvement in the memory and cognition function.

The present invention contemplates the use of a Hh agonist, preferably in pharmaceutical compositions as described below, for the treatment or prophylaxis of depression, panic disorder, obsessive compulsive disorders, anxiety, pain (in particular chronic pain), psychoactive substance abuse, migraine headaches, social anxiety/phobic disorder, and posttraumatic stress syndrome, as well as an appetite suppressant. For any of these purposes, treatment includes partial or total alleviation of one or more symptoms of a condition, and prophylaxis includes delaying the onset of or reducing the severity of one or more symptoms of a condition. Although the methods described herein are expected to be effective in any animal, particularly mammals, treatment of humans is preferred in certain embodiments.

Another aspect of the present invention provides the pharmaceutical compositions which stimulate the Hh signaling pathway. The pharmaceutical compositions comprise a Hh polypeptide or its functional equivalent, or an agonist of Hh activity. The pharmaceutical compositions may also comprise an antagonist of the negative feedback system or of repressive elements of the Hh pathway. The pharmaceutical compositions may further comprise additional therapeutic agents, such as neuronal growth factors or neurotrophic factors.

In one embodiment, the agent to stimulate the pathway in the methods of the present invention is a Hh polypeptide or its functional equivalent. Preferably, the agent is a Hh polypeptide. More preferably, the agent is a Shh polypeptide. In one embodiment, the agent is a fragment of a Hh polypeptide. More preferably, it is an N-terminal fragment containing a region that binds to a receptor for a Hh polypeptide. Even more preferably, the fragment is a 19 kDa N-terminal fragment of a human Hh polypeptide. In another embodiment, the agent is a polypeptide which shares at least 60, 70, 80, or 90% amino acid sequence homology with any of the Hh amino acid sequences depicted as SEQ ID NOs: 10 to 19.

In certain embodiments, the Hh polypeptides used to practice the methods of the present invention are modified by a lipophilic moiety or moieties at one or more internal sites of the mature, processed extracellular domain, and may or may not be also derivatized with lipophilic moieties at the N or C-terminal residues of the mature polypeptide. In other embodiments, the polypeptide is modified at the C-terminal residue with a hydrophobic moiety other than a sterol. In still other embodiments, the polypeptide is modified at the N-terminal residue with a cyclic (preferably polycyclic) lipophilic group. Various combinations of the above are also contemplated. For exemplary modifications of a polypeptide including a Hh polypeptide, see U.S. application Ser. No. 09/579,680, the disclosure of which is incorporated by reference herein in its entirety.

In another embodiment of the present invention, the agent to practice the method of invention is a small molecule agonist. Preferably, the small molecule is a compound having a molecular weight less than about 2500 amu, even more preferably less than about 1500 amu.

In one embodiment, the agent is an anti-idiotypic antibody against an antibody to a protein of Hh family. Such an anti-idiotypic antibody mimics the action of a Hh polypeptide.

In certain embodiments, the methods include co-administration of the agent with one or more of a neuronal growth factor, a neuronal survival factor, or a neuronal tropic factor.

In other embodiments, the subject method can be carried out by administering a gene activation construct, wherein the gene activation construct is deigned to recombine with a genomic hh gene of the patient to provide a heterologous transcriptional regulatory sequence operatively linked to a coding sequence of the hh gene.

In other embodiments, the Hh agonist of the present invention is an RNAi construct, wherein the construct inhibits the expression of a negative regulatory element in the Hh signaling pathway, causing the release of repression or suppression of the Hh signaling and resulting in the activation of the pathway.

In still other embodiments, the subject method can be practiced with the administration of a gene therapy construct encoding a Hh polypeptide or its equivalent. For instance, the gene therapy construct can be provided in a composition selected from a recombinant viral particle, a liposome, and a poly-cationic nucleic acid binding agent.

In yet another aspect, the invention provides a method for conducting a pharmaceutical business by determining an appropriate formulation and dosage of a Hh agonist in the treatment of depression or another behavioral or emotional disorder, and licensing, to a third party, the rights for further development and sale of the formulation.

In still a further aspect, the invention relates to a method for conducting a pharmaceutical business, by determining an appropriate formulation and dosage of a Hh agonist in the treatment of depression or another behavioral or emotional disorder, conducting therapeutic profiling of identified formulations for efficacy and toxicity in animals, and providing a distribution network for selling a preparation as having an acceptable therapeutic profile. In certain embodiments, the method further includes an additional step of providing a sales group for marketing the preparation to healthcare providers.

### DETAILED DESCRIPTION OF THE INVENTION

I. Overview

The invention is based on the physiological functions of the hedgehog (hh) family of proteins, which is known to play a crucial role in development and differentiation of embryonic stem cells of animals. Recently it has been observed that a Hedgehog (Hh) polypeptide also plays a role in an adult animal, guiding differentiation and migration of stems cells into various functional cells in the appropriate locations. Hh polypeptides play a role in the neurogenesis, neuronal differentiation, and migration of neuronal stem cells in an animal's central nervous system, and also in the maintenance and protection of neurons. See, for example, Bezard, E. et al. (2003) FASEB J. express article 10.1096/fj.03-0291 je, published online, Pascuala, O. et al. (2002) J. Physiol.—Paris 96: 135-166, and Machold, R. et al. (2003) Neuron 39: 937-950, the disclosures of which are incorporated by reference herein.

It has recently been observed that various anti-depressants stimulate neurogenesis in hippocampus and that the neurogenesis contributes to the effect of the anti-depressants. Antidepressants such as fluoxetine or imipramine increase neurogenesis in the dentate gyrus of the rat hippocampus. When this neurogenesis was disrupted by irradiating the hippocampus, the test subject mice no longer responded to antidepressant treatment, as measured by the novelty suppressing feeding (NSF) test and by the chronic unpredictable stress paradigm. In addition, knock-out mice lacking 5-HT_{1A} receptors are non-responsive to fluoxetine, a serotonin selective reuptake inhibitor, but respond to imipramine and desipramine, tricyclic antidepressants, indicating there are two independent molecular pathways. See Santarelli, L. et al. (2003) Science 301:805-809. Therefore, a Hh polypeptide or a Hh agonist, which stimulates neurogenesis and differentiation, is expected to act as an antidepressant.

In addition, Hh polypeptides seem to be directly involved in the regulation of electrical activity of subthalmic nucleus (STN) neurons of adult animals. Within minutes of application of an N-terminal fragment of the Shh polypeptide, the electrical activity of a subset of STN neurons in rat brain slices are inhibited, reducing the synaptic transmission. The STN is a key element of the basal ganglia, which is now recognized to play a role in the emotional and cognitive activities in addition to controlling voluntary and involuntary movements. Therefore, the Hh polypeptide is implicated to directly participate in regulation of emotional and cognitive response in a subject. Further, application of lipid modified form of sonic hedgehog (Shh) to a brainstem slice preparation reversibly modified the activity of adult nucleus tractus solitarius (NTS) neurons, causing an inhibition followed by a delayed burst of action potentials. The NTS is a brainstem structure involved in regulation of respiratory, cardio-vascular and alimentary functions. Because Shh is produced in an area of the brain immediately adjacent to the NTS, Shh may exert a neuromodulatory function in the adult NTS. See Pascuala, above.

Many neurological disorders are associated with degeneration of discrete populations of neuronal elements and may be treatable with a therapeutic regimen which includes a Hh agonist. It has recently been observed that patients suffering from severe depression exhibit atrophy of the hippocampus. Treatment of patients suffering from such degenerative conditions can include the application of Hh polypeptides, or agents which mimic their effects, in order to control, for example, differentiation and apoptotic events which give rise to loss of neurons (e.g., to enhance survival of existing neurons) as well as promote differentiation and repopulation by progenitor cells in the area affected. Treatment of a subject animal with a Hh agonist before an assault on the CNS in a controlled laboratory experiment results in smaller lesions and retention of the neuronal volume.

Dementia and memory loss is seen in several degenerative diseases characterized by the death of neurons in various parts of the central nervous system, especially the cerebral cortex. Some forms of dementia are associated with degeneration of the thalamus or the white matter underlying the cerebral cortex. For example, Alzheimer's disease (AD) is associated with deficits in several neurotransmitter systems, both those that project to the neocortex and those that reside with the cortex. For instance, the nucleus basalis in patients with AD has been observed to have a profound (75%) loss of neurons compared to age-matched controls. Recently, it has been shown that in the hippocampus of human patients with AD, neurogenesis is upregulated, as shown by the increased expression of immature neuronal marker proteins. Jin, K. et al. (2003) Proc. Natl. Acad. Sci. USA, Early edition, www.pnas.org/cgi/doi/10.1073/pnas.2634794100. Such increased neurogenesis may be the body's natural defense against pathological cell loss and may be applicable to other degenerative diseases that exhibit loss of neurons. Augmenting and enhancing the neurogenesis is expected to be a viable treatment option.

Although AD is by far the most common form of dementia, several other disorders can produce dementia, including vascular, or multi-infarct, dementia, Lewy body dementia, Pick's disease, Huntington's disease, progressive supranuclear palsy, focal cortical atrophy syndromes (such as primary aphasia), metabolic-toxic dementias (such as chronic hypothyroidism or B12 deficiency), and dementias caused by infections (such as syphilis, neuroAIDS or chronic meningitis). In some diseases, the cognitive dysfunction results from the isolation of cortical areas by the degeneration of efferents and afferents. Huntington's disease involves the degeneration of intrastriatal and cortical cholinergic neurons and GABAergic neurons. Pick's disease is a severe neuronal degeneration in the neocortex of the frontal and anterior temporal lobes, sometimes accompanied by death of neurons in the striatum.

The methods of the present invention are amenable also to the treatment of disorders of the cerebellum which result in hypotonia or ataxia, such as those lesions in the cerebellum which produce disorders in the limbs ipsilateral to the lesion. For instance, a preparation of a hh homolog can used to treat a restricted form of cerebellar cortical degeneration involving the anterior lobes (vermis and leg areas) such as is common in alcoholic patients.

The present invention is generally directed to the methods and compositions for treatment of emotional and behavioral disorders, such as depression, and for enhancement of cognition and memory and treatment of neurological disorders that involves loss of memory and cognitive functions, such as various dementias. Here we provide methods and compositions for treatment of such diseases based on the stimulation of the Hh signaling pathway. The methods of invention may be practiced using a Hh polypeptide or its functional equivalents, including peptide fragments and mutant proteins. The methods may also be practiced using small peptides, peptidomimefics, or organic molecules, as well as antibodies. The methods also may be practiced by carrying out gene therapy, i.e., introducing certain gene constructs, comprising, for example, a hedgehog, smoothened, or gli-1 gene, to the subject so that a functional protein is produced from within the subject cells.

II. Definitions

For convenience, certain terms employed in the specification, examples, and appended claims are collected here.

As used herein, the terms “agent” and “compound” include both protein and non-protein moieties. An agent may be a small organic molecule, a polypeptide, a protein, a peptide complex, a peptidomimefic, a non-peptidyl agent, or a polynucleotide.

As used herein, “ameliorates” means alleviate, lessen, or and decrease the extent of a symptom or decrease the number of occurrence of episodes of a disease manifestation.

As used herein, “antibody” means an immunoglobulin molecule comprising two heavy chains and two light chains and which recognizes an antigen. The immunoglobulin molecule may derive from any of the commonly known classes, including but not limited to IgA, secretory IgA, IgG and IgM. IgG subclasses are also well known to those in the art and include but are not limited to human IgG1, IgG2, IgG3 and IgG4. It includes, by way of example, both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. Optionally, an antibody can be labeled with a detectable marker. Detectable markers include, for example, radioactive or fluorescent markers. Antibodies may also be modified by coupling them to other biologically or chemically functional moieties such as cross-linking agents or peptides.

The terms “antidepressant,” “antidepressants,” and “antidepressant moiety” refer to CNS active moieties or prodrug forms thereof, whose main effect is to prevent, treat or ameliorate acute or chronic depression. Exemplary antidepressants include bicyclic antidepressants, such as caroxazone, fencamine, indalpine, indeloxazine HCl, nomifensine, oxitriptan (L-5HTP), paroxetine and sertraline; hydrazides, such as benmoxine, iproclozide, iproniazid, isocarboxazid, octamoxin and phenelzine; pyrrolidones, such as rolicyprine, rolipram and sertindole; tetracyclic antidepressants, such as maprotiline; tricyclic antidepressants such as amoxapine, demexiptiline, desipramine, metapramine, nortiptaline, opipramol, propizepine, protriptyline and tianeptine; and other antidepressants, such as adrafinil, benactyzine, dioxadrol, duloxetine, febarbamate, fenpentadiol, fluvoxamine, hematoporphyrin, hypericine, levophacetoperane, milnacipran, minaprine, moclobemide, pyrisuccideanol, roxindole, sulpiride, toloxatone, tranylcypromine, 1-tryptophan, venlafaxine and viloxazine.

The terms “antipsychotic,” “antipsychotics,” and “antipsychotic moieties” are used interchangeably and refer to CNS active moieties, or prodrug forms thereof, whose main effect is to prevent, treat or ameliorate an acute or chronic psychotic. Exemplary antipsychotics include benzamides, such as amisulpride, nemonapride and sulpiride; benzisoxazoles; butyrophenones, such as benperidol, bromperidol, droperidol, haloperidol, moperone, pipamperone, spiperone, timiperone and trifluperidol; phothiazines, such as acetophenazine, carphenazine, dixyrazine, fluphenazine, pericyazine, perimethazine, perphenazine, piperacetazine and pipotiazine; thioxanthenes, such as clopenthixol and flupentixol; other tricyclic antipsychotic compounds, such as carpipramine, clocapramine, mosaprimine, olanzapine, risperidone, 9-hydroxy-risperidone, opipramol and seroquel; and other antipsychotics, such as aripiprazole, buramate, penfluridol, pimozide and ziprasidone.

The terms “anxiolytic,” “anxiolytics,” “antianxiety moiety,” “anxiolytic moiety” and “antianxiety moiety” refer to CNS active moieties or prodrug forms thereof, whose primary function is to alleviate, prevent, treat or ameliorate acute or chronic anxiety disorders. Severe anxiety disorders include general anxiety disorder (GAD), panic disorders, phobias, obsessive-compulsive disorder (OCD) and post-traumatic stress disorder (PSTD). Exemplary anxiolytics include arylpiperazines, such as enciprazine and flesinoxan; benzodiazepine derivatives, such as chlordiazepoxide, clorazepate, flutazolam, lorazepam, mexazolam, diazepam, alprazolam, clonazepam, chlordiazepoxide, nordazepam and oxazepam; carbamates, such as emylcamate, hydroxyphenamate, meprobamate, phenprobamate and tybamate; and other anxiolytic compounds, such as benzoctamine, glutamic acid, hydroxyzine, mecloralurea, mephenoxalone, propranolol, atenolol, buspirone, valproate, neurontin, carbamazepine, and oxanamide and selective serotonin reuptake inhibitors (SSRI's), such as fluoxetine, fluvoxamine, indalpine, indeloxazine HCl, milnacipran, paroxetine and sertralin.

The term “anxiety disorders” includes, but is not limited to obsessive-compulsive disorder, psychoactive substance anxiety disorder, post-traumatic stress disorder, generalized anxiety disorder, social anxiety disorder, phobia, social phobia, anxiety disorder NOS, and organic anxiety disorder.

The term “autistic disorder” as used herein means a condition characterized as an Autistic Disorder in the DSM-IV-R as category 299.xx, including 299.00, 299.80, and 299.10, preferably 299.00.

The term “bipolar disorder” as used herein refers to a condition characterized as a Bipolar disorder, in the DSM-IV-R as category 296.xx, including both Bipolar Disorder I and Bipolar Disorder II.

As used herein, the term “dsRNA” refers to small interference RNA (siRNA) molecules, or other RNA molecules including a double stranded feature and able to be processed to siRNA in cells, such as hairpin RNA moieties.

The term “ED₅₀” means the dose of a drug which produces 50% of its maximum response or effect.

An “effective amount” of, e.g., a Hh agonist, with respect to the subject method of treatment, refers to an amount of the agonist in a preparation which, when applied as part of a desired dosage regimen brings about, e.g., a change in the rate of cell proliferation and/or the state of differentiation of a cell and/or rate of survival of a cell according to clinically acceptable standards for the disorder to be treated or the effect desired, such as enhanced memory or cognition.

The term “excessive aggression” as used herein refers to a condition characterized by aggression that is so excessive that it interferes with the individual's daily functions, relationships, and may threaten the safety of the individual, for example in a situation in which violent suicide is contemplated. The excessive aggression which may be treated using the method claimed herein is independent of a psychotic condition and not directly related to the consumption of a drug or other substance.

The term “gain-of-function,” as it refers to genes inhibited by the subject RNAi method, refers to a increase in the level of expression of a gene when compared to the level in the absence of RNAi constructs.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The term “Hh agonist” refers to an agent which potentiates or recapitulates the bioactivity of Hh, such as to activate transcription of target genes, especially gli-1. The term “Hh agonist” as used herein refers not only to any agent that may act by directly activating the normal function of a Hh or smoothened polypeptide, but also to any agent that activates the Hh signaling pathway, including any agent that relieves repression or suppression of a negative regulatory element of the pathway, such as the Patched protein. Thus, an Hh agonist may be an inhibitor of a negative regulatory element of the Hh signaling pathway. As used herein, the term “Hh agonist” includes RNA interference (RNAi) modulators that suppress the expression of negative-control elements within the Hh signaling pathway. Preferred Hh agonists can be used to mimic or enhance the activity or effect of Hh polypeptide in a smoothened-dependent manner. Another type of preferred Hh agonists disrupt the association of the Smoothened and Patched polypeptides, relieving the repressive effect of Patched and activating the Hh pathway.

The term “Hh polypeptide” refers any protein expressed from a gene belonging to the hh gene family, its mutants and functionally equivalent polypeptides. A “gene family” means a group of genes that share a common function and exhibit common sequence homology.

The term “hh RNAi agonist” refers to an RNAi agent that inhibits the bioactivity of a hh signaling component (for example gli-3), such that it represses the expression of the target hh signaling component which normally acts as a suppressor or a repressor of the hh signaling. For example, certain preferred hh RNAi agonists can be used to overcome a ptc gain-of-function and/or a gli-3 gain-of-function. Other preferred RNAi agonists can be used to relieve suppression in hh signal transduction. An RNAi agonist may be directed to a gene encoding a protein in the Hh signaling pathway. In most cases, the RNAi agonist would inhibit the activity of the target protein by, for example, decreasing production of a protein encoded by a gene in the Hh pathway which negatively regulates the pathway, thus upregulating Hh signaling.

As used herein, “inhibits” means that the amount is reduced as compared with the amount that would occur in a control sample. In a preferred embodiment, inhibits means that the amount is reduced by more than 50%, even more preferably by more than 75% or even 100%.

As used herein, “instruction material” means a document or recorded media including a written or audible instruction for the use of a pharmaceutical composition. An instruction material includes a label on a bottle, a paper inserted a box, printing on the box or carton, instructions provided by a website at an address given in any of these locations, etc.

The term “LD₅₀” means the dose of a drug which is lethal in 50% of test subjects.

As used herein, the phrase “mediates RNAi” refers to the ability to distinguish which RNAs are to be degraded by the RNAi process, e.g., degradation occurs in a sequence-specific manner rather than by a sequence-independent dsRNA response, e.g., a PKR response.

The term “non-Alzheimer dementia” as used herein means any form of dementia and mental impairment characterized by deterioration of intellectual faculties, such as memory, concentration, and judgment, resulting from an organic disease or a disorder of the brain, not accompanied with other hallmarks of Alzheimer's disease. It is sometimes accompanied by emotional disturbance and personality changes.

The term “preventing” is art-recognized, and when used in relation to a condition, such as recurrence or onset of a disease such as depression, a syndrome complex such as dementia or any other medical condition, is well understood in the art, and includes administration of a composition which reduces the frequency of, or delays the onset of, symptoms of a medical condition in a subject relative to a subject which does not receive the composition. Thus, prevention of depression includes, for example, reducing the recurrence of depressive episodes in a population of patients receiving a prophylactic treatment relative to an untreated control population, and/or delaying the onset of depression in a treated population compared to untreated population. Prevention of memory impairment includes, for example, reducing the number of episodes of failed recollection in a population of patients receiving a prophylactic treatment relative to an untreated control population, and/or delaying the appearance of memory deficiency in a treated population versus an untreated control population, e.g., by a statistically and/or clinically significant amount. Prevention of deficiency in cognitive function includes, for example, reducing the number of episodes of cognitive impairment in a treated population versus an untreated control population, and/or delaying the onset of symptoms of cognitive impairment in a treated population versus an untreated control population.

The term “psychotic condition” as used herein means pathologic psychological conditions which are psychoses or may be associated with psychotic features. Such conditions include, but are not limited to the psychotic disorders which have been characterized in the DSM-IV-R, Diagnostic and Statistical Manual of Mental Disorders, Revised, 4th Ed. (1994), including schizophrenia and acute mania. The DSM-IV-R was prepared by the Task Force on Nomenclature and Statistics of the American Association, and provides clear descriptions of diagnostic categories. The skilled artisan will recognize that there are alternative nomenclatures, nosologies, and classification systems for pathologic psychological conditions and that these systems evolve with medical scientific progress.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “siRNA” stands for a small interfering RNA.

The term “small molecule” refers to a compound having a molecular weight less than about 2500 amu, preferably less than about 2000 amu, even more preferably less than about 1500 amu, still more preferably less than about 1000 amu, or most preferably less than about 750 amu.

As used herein, “statistically normal range” means scoring no less than 20 percentile in a test or assay accepted by one skilled in the art as reproducible and representative of the tested quantity. A score is considered to be within a statistically normal range when, within a given population, at least 20% of the scores obtained in the same test are lower than the score being considered. Put differently, assuming a normal distribution, a score that falls below 0.84 standard deviation of the mean of the comparable test scores is considered outside the statistically normal range.

A “subject” or “patient” to be treated by the subject method can mean either a human or non-human animal.

As used herein, “treating” means either slowing, stopping or reversing the progression of the disorder. In the preferred embodiment, “treating” means reversing the progression to the point of eliminating the disorder.

The term “acylamino” is art-recognized and refers to a moiety that can be represented by the general formula: 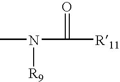
wherein R₉ is as defined above, and R′₁₁ represents a hydrogen, an alkyl, an alkenyl or —(CH₂)ₘ—R₈, where m and R₈ are as defined above.

Herein, the term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

The terms “alkenyl” and “alkynyl” refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethylthio, and the like.

The terms “amine” and “amino” are art-recognized and refer to both unsubstituted and substituted amines, e.g., a moiety that can be represented by the general formula: 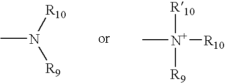
wherein R₉, R₁₀ and R₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In still more preferred embodiments, the term ‘amine’ does not encompass amides, e.g., wherein one of R₉ and R₁₀ represents a carbonyl. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “amido” is art-recognized as an amino-substituted carbonyl and includes a moiety that can be represented by the general formula: 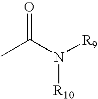
wherein R₉, R₁₀ are as defined above. Preferred embodiments of the amide will not include imides which may be unstable.

The term “aralkyl”, as used herein, refers to an alkyl group substituted with an aryl group (e.g., an aromatic or heteroaromatic group).

The term “aryl” as used herein includes 5-, 6-, and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The term “carbocycle”, as used herein, refers to an aromatic or non-aromatic ring in which each atom of the ring is carbon.

The term “carbonyl” is art-recognized and includes such moieties as can be represented by the general formula: 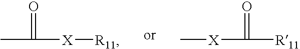
wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R₁₁′ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Preferred heteroatoms are boron, nitrogen, oxygen, phosphorus, sulfur and selenium.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

A “phosphonamidite” can be represented in general formula: 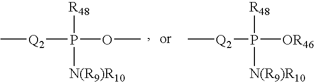
wherein R₉ and R₁₀ are as defined above, Q₂ represents O, S or N, and R₄₈ represents a lower alkyl or an aryl, Q₂ represents O, S or N.

A “phosphoramidite” can be represented in general formula: 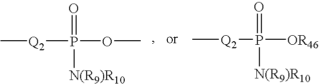
wherein R₉ and R₁₀ are as defined above, and Q₂ represents O, S or N.

A “phosphoryl” can in general be represented by the formula: 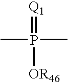
wherein Q₁ represented S or O, and R₄₆ represents hydrogen, a lower alkyl or an aryl. When used to substitute, for example, an alkyl, the phosphoryl group of the phosphorylalkyl can be represented by the general formula: 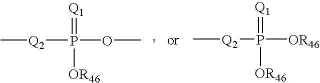
wherein Q₁ represented S or O, and each R₄₆ independently represents hydrogen, a lower alkyl or an aryl, Q₂ represents O, S or N. When Q₁ is an S, the phosphoryl moiety is a “phosphorothioate”.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R₈, m and R₈ being defined above.

As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety that can be represented by the general formula: 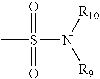
in which R₉ and R₁₀ are as defined above.

The term “sulfate” is art recognized and includes a moiety that can be represented by the general formula: 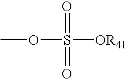
in which R₄₁ is as defined above.

The term “sulfonamido” is art recognized and includes a moiety that can be represented by the general formula: 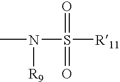
in which R₉ and R₁₁′ are as defined above.

The term “sulfonate” is art-recognized and includes a moiety that can be represented by the general formula: 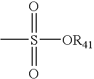
in which R₄₁ is an electron pair, hydrogen, alky, cycloalkyl, or aryl.

The terms “sulfoxido” or “sulfinyl”, as used herein, refers to a moiety that can be represented by the general formula: 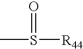
in which R₄₄ is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

III. Exemplary Methods and Compositions

Methods of Treatment

Activating the Hh signaling pathway stimulates neurogenesis, differentiation and migration of neuronal stem cells. A Hh polypeptide also directly modulates synaptic electrical neurotransmission. Therefore, according to the present invention, a Hh agonist is used in methods to treat various disorders and conditions that benefit from increased neuronal growth and differentiation, and from modulated synaptic activity.

One aspect of the present invention provides methods for modulating activity of CNS of a mammal by stimulating the neuronal stem cells via a Hh signaling pathway, thereby promoting differentiation and migration of the neuronal stem cells. The methods of the present invention comprise administering a Hh agonist to a subject experiencing certain deficits in CNS neuronal functions or a subject that benefits from enhancement of certain CNS functions.

More specifically, the present invention provides methods for treating behavioral and/or emotional disorders by modulating the activity of central nervous system via the Hh signaling pathway.

The present invention contemplates the use of a Hh agonist, preferably in pharmaceutical compositions as described below, for the treatment or prophylaxis of emotional disorders such as depression, panic disorder, obsessive compulsive disorders, anxiety, and social anxiety/phobic disorder. For any of these purposes, treatment includes partial or total alleviation of one or more symptoms of a condition, and prophylaxis includes delaying the onset of or reducing the severity of one or more symptoms of a condition.

A specific aspect of the present invention is treatment of depression. Anti-depressant small molecules have been shown to stimulate neurogenesis in hippocampus and that the neurogenesis contributes to the effect of the anti-depressants. A Hh agonist stimulates neurogenesis in the hippocampus and is expected to to show a similar effect compared to known antidepressants.

Another aspect of the present invention provides methods of enhancement of cognitive function and/or memory function of a subject. An aspect of the present invention also provides enhancement of cognition, which is additionally contemplated to treat diseases that exhibit associated dementia, and to alleviate symptoms of these diseases and other disorders such as depression which exhibit degradation of memory and cognitive functions. Still another aspect of the invention relates to the use of Hh agonists for prophylactically preventing the occurrence of learning and/or memory defects in a subject, and thus, altering the learning ability and/or memory capacity of the subject. In certain embodiments, the subject method can be used to treat patients who have been diagnosed as having or being at risk of developing disorders in which diminished declarative memory is a symptom, e.g., as opposed to procedural memory. As a result, the methods of the present invention may be useful for preventing memory impairment. Contemplated causes of memory impairment include toxicant exposure, brain injury, age-associated memory impairment, mild cognitive impairment, epilepsy, mental retardation in children, and dementia resulting from a disease, such as in certain cases of Parkinson's disease, AIDS, head trauma, Huntington's disease, Pick's disease, Creutzfeldt-Jakob disease, post cardiac surgery, Downs Syndrome, Anterior Communicating Artery Syndrome, and other symptoms of stroke. Yet another aspect of the present invention provides methods of treatment of disorders which are accompanied by neuronal cell loss or lesion, by stimulating the neuronal stem cells to differentiate and migrate to the site of the damage. Such differentiation and migration can be promoted by activating the Hh signaling pathway by various agents. In addition, the present invention may be useful in enhancing memory in normal individuals.

The most common cause of dementia in the elderly is Alzheimer's disease (AD). AD is an etiologically unknown, non-infectious neurological disorder that shows progressive dementia. About 3 to 5% of people over 65 suffer from AD. While the definitive characteristic of AD is a postmortem observation of amyloid plaques and neurofibrillary tangles (malformations within nerve cells) in the brain of a patient, guidelines have been established to aid the diagnosis of AD in a living patient. The National Institute of Neurological and Communicative Disorders and Stroke—Alzheimer's Disease and Related Disorders Association (NINCDS-ADRDA) has devised a list of indicative symptoms to diagnose AD. There are several criteria for likelihood of the subject suffering from AD.
A. Criteria for the clinical diagnosis of Probable AD
   1. Dementia established by
      a1. Clinical examination, and
      a2. Documented by the MMSE, BDS, or other similar examination, and
      a3. Confirmed by neuropsychological test
      b. Deficits in two or more areas of cognition
      c. Progressive worsening of memory and other cognitive functions
      d. No disturbance of consciousness
      e. Onset between ages 40 and 90, most often after age 65
      f. Absence of systemic disorders or other brain diseases that could account for the progressive deficits in memory and cognition
   2. The diagnosis of probable AD is supported by
      a. Progressive deterioration of specific cognitive functions such as language (aphasia), motor skills (apraxia), and perception (agnosia);
      b. Impaired activities of daily living and altered patterns of behavior
      c. Family history of similar disorders, particularly if neuropathologically confirmed
      d. Laboratory results of
         d1. Normal lumbar puncture as evaluated by standard techniques;
         d2. Normal pattern or nonspecific EEG changes, such as increased slow wave activity;
      e. Evidence of cerebral atrophy on CT with progression documented by serial observation
   3. Other clinical features consistent with probable AD after exclusion of other causes of dementia
      a. Plateaus in the course of progression of the illness
      b. Associated symptoms of depression, insomnia, incontinence, delusions, illusions, hallucinations, catastrophic verbal, emotional, or physical outburst, sexual disorders, and weights loss
      c. Other neurological abnormalities in some patients, especially those with more advanced disease, including motor signs such as increased muscle tone, myoclonus, or gait disorder
      d. Seizures in advanced disease
      e. CT normal for age
   4. Features that make the diagnosis of probable AD uncertain or unlikely
      a. Sudden, apoplectic onset
      b. Focal neurologic findings such as hemiparesis, sensory loss, visual field deficits, and incoordination early in the course of the illness
      c. Seizures or gait disturbances at the onset of symptoms or very early in the course of the illness
B. Diagnosis of Possible AD
   1. May be made on the basis of the dementia syndrome;
      a. In the absence of other neurologic, psychiatric, or systemic disorders sufficient to cause dementia; and
      b. in the presence of variations in the onset, presentation, or clinical course
   2. May be made in the presence of a second systemic or brain disorder sufficient to produce dementia but considered to be the cause of the dementia
   3. Should be used in research studies when a single gradually progressive severe cognitive deficit is identified in the absence of another identifiable cause
C. Criteria for diagnosis of Definite AD
   1. Clinical criteria for probable AD
   2. Histopathologic evidence obtained from biopsy or autopsy
   3. Subtype classification for research purposes
   4. Familial occurrence
   5. Onset before age 65
   6. Presence of trisomy-21
   7. Coexistence of other relevant conditions such as PD

The DSM-IV criteria of AD are as follows:
A. The development of multiple cognitive deficits manifested by both:
   1. Memory impairment (impaired ability to learn new information or to recall previously learned information).
   2. One (or more) of the following cognitive disturbances:
      a. Aphasia (language disturbance)
      b. Apraxia (impaired ability to carry out motor activities despite intact motor function)
      c. Agnosia (failure to recognize or identify objects despite intact sensory function)
      d. Disturbance in executive functioning (i.e., planning, organizing, sequencing, abstracting)
B. The cognitive deficits in A1 and A2 each cause significant impairment in social or occupational functioning and represent a significant decline from a previous level of functioning.
C. The course is characterized by gradual onset and continuing cognitive decline.
D. The cognitive deficits in criteria A1 and A2 are not due to any of the following:
   1. Other central nervous system conditions that cause progressive deficits in memory and cognition (e.g., cerebrovascular disease, Parkinson's disease, Huntington's disease, subdural hematoma, normal-pressure hydrocephalus, brain tumor)
   2. Systemic conditions that are known to cause dementia (e.g., hypothyroidism, hyperthyroidism, vitamin B 12 or folic acid deficiency, niacin deficiency, hypercalcemia, neurosyphilis, HIV infection)
   3. Substance-induced conditions
E. The deficits do not occur exclusively during the course of delirium
F. The disturbance is not better accounted for by another Axis I disorder (e.g., major depressive disorder, schizophrenia)

Hallmarks of Alzheimer's disease include progressive nature of dementia, characteristic positron emission tomography showing reduced 2FDG metabolism in parietal and temporal lobe association and posterior cingulate cortices. Reductions are usually bilateral, yet there often is an asymmetry in the severity or the extent of hypometabolism. Patients with advanced clinical symptoms often demonstrate reduced metabolism in the prefrontal association cortices as well. Metabolism is relatively spared in primary sensory and motor cortical regions, including the somatomotor, auditory and visual cortices. Subcortical structures, including the basal ganglia, thalamus, brainstem and cerebellum, are also preserved in typical AD. The overall distribution of metabolism in AD reflects in part the known regional losses of neurons and synapses but likely also includes effects of cortical disconnection resulting in reduced afferent input to the association areas. Additionally, increase in biomarkers such as total tau, and phosphorylated tau in the cerebrospinal fluid aids the diagnosis of Alzheimer's disease. Genetic factors that increase the risk of Alzheimer's, such as being homozygous for allele 4 of ApoE protein, support the diagnosis. For a recent review of biological markers of AD, see Frank, R. A. et al. (2003) Neurobiol. Aging 24:521-536, the disclosure of which is incorporated herein by reference in its entirety.

In various embodiments, the present invention contemplates modes of treatment and prophylaxis which utilize one or more Hh agonists. These agonists may be useful for decreasing the occurrence of learning and/or memory defects in an organism, and thus maintaining the learning ability and/or memory function of the organism. In other embodiments, the preparations of the present invention can be used simply to enhance normal memory function.

The methods and compositions of the present invention can be used for the treatment of movement disorders. Hh agonists can be used to treat patients suffering from ataxia, corticobasal ganglionic degeneration (CBGD), dyskinesia, dystonia, tremors, hereditary spastic paraplegia, Huntington's disease, multiple sclerosis, multiple system atrophy, myoclonus, Parkinson's disease, progressive supranuclear palsy, restless legs syndrome, Rett syndrome, spasticity, Sydenham's chorea, other choreas, athetosis, ballism, stereotypy, tardive dyskinesia/dystonia, tics, Tourette's syndrome, olivopontocerebellar atrophy (OPCA), diffuse Lewy body disease, hemibalismus, hemi-facial spasm, restless leg syndrome, Wilson's disease, stiff man syndrome, akinetic mutism, psychomotor retardation, painful legs moving toes syndrome, a gait disorder, a drug-induced movement disorder, or other movement disorder.

The methods and compositions of the present invention can be used to treat or otherwise reduce the severity of behavioral disorders such as attention deficit disorder (ADD), attention deficit hyperactivity disorder (ADHD), and cognitive disorders such as dementias (including age related dementia, HIV-associated dementia, AIDS dementia complex (ADC), HIV encephalopathy and senile dementia).

Characteristics of ADHD have been demonstrated to arise in early childhood for most individuals. This disorder is marked by chronic behaviors lasting at least six months with an onset often before seven years of age. At this time, four subtypes of ADHD have been defined as follows:
1. Inattentive type
2. Hyperactive/impulsive type
3. Combined type
4. Not otherwise specified is defined by an individual who demonstrates some characteristics but an insufficient number of symptoms to reach a full diagnosis. These symptoms, however, disrupt everyday life.

The criteria for diagnosing ADHD, according to the American Psychiatric ion Diagnostic and Statistical Manual (DSM-IV), include:
A. Either (1) or (2) of the following:
(1). six (or more) of the following symptoms of inattention have persisted for at least 6 months to a degree that is maladaptive and inconsistent with developmental level:
   Inattention
   (a) often fails to give close attention to details or makes careless mistakes in schoolwork, work, or other activities
   (b) often has difficulty sustaining attention in tasks or play activities
   (c) often does not seem to listen when spoken to directly
   (d) often does not follow through on instructions and fails to finish schoolwork, chores, or duties in the workplace (not due to oppositional behavior or failure to understand instructions)
   (e) often has difficulty organizing tasks and activities
   (f) often avoids, dislikes, or is reluctant to engage in tasks that require sustained mental effort (such as schoolwork or homework).
   (g) often loses things necessary for tasks or activities (e.g. toys, school assignments, pencils, books, or tools)
   (h) is often easily distracted by extraneous stimuli
   (i) is often forgetful in daily activities
(2). six (or more) of the following symptoms of hyperactivity-impulsivity have persisted for at least 6 months to a degree that is maladaptive and inconsistent with developmental level:
   Hyperactivity
   (a) often fidgets with hands or feet or squirms in seat
   (b) often leaves seat in classroom or in other situations in which remaining seated is expected
   (c) often runs about or climbs excessively in situations in which it is inappropriate (in adolescents or adults, may be limited to subjective feelings of restlessness)
   (d) often has difficulty playing or engaging in leisure activities quietly
   (e) is often “on the go” or often acts as if “driven by a motor”
   (f) often talks excessively
   Impulsivity
   (g) often blurts out answers before questions have been completed
   (h) often has difficulty awaiting turn
   (i) often interrupts or intrudes on others (e.g., butts into conversations or games)
B. Some hyperactive-impulsive or inattentive symptoms that caused impairment were present before age 7 years.
C. Some impairment from the symptoms is present in two or more settings (e.g. at school [or work] and at home).
D. There must be clear evidence of clinically significant impairment in social, academic, or occupational functioning.
E. The symptoms do not occur exclusively during the course of a Pervasive Developmental Disorder, Schizophrenia, or other Psychotic Disorder and are not better accounted for by another mental disorder (e.g. Mood Disorder, Anxiety Disorder, Dissociative Disorder, or a Personality Disorder)

The methods and compositions of the present invention can be used as part of therapy for treating patients suffering from autistic disorders.

The methods and compositions of the present invention can be used as part of therapy for patients suffering from dyssomnias, parasomnias, sleep disorders associated with medical or psychiatric conditions, or other sleep disorders. In certain preferred embodiments, the dyssomnias are selected from intrinsic sleep disorders, extrinsic sleep disorders, and circadian rhythm sleep disorders. Examples of intrinsic sleep disorders include psychophysiological insomnia, sleep state misperception, idiopathic insomnia, narcolepsy, recurrent hypersomnia, idiopathic hypersomnia, posttraumatic hypersomnia, obstructive sleep apnea syndrome, central sleep apnea syndrome, central alveolar hypoventilation, periodic limb movement disorder, restless leg syndrome (RLS), etc. Examples of extrinsic sleep disorders include inadequate sleep hygiene, environmental sleep disorder, altitude insomnia, adjustment sleep disorder, insufficient sleep syndrome, limit-setting sleep disorder, sleep-onset association disorder, food allergy insomnia, nocturnal eating/drinking syndrome, hypnotic-dependent sleep disorder, stimulant-dependent sleep disorder, alcohol-dependent sleep disorder, toxin-induced sleep disorder, etc. Examples of circadian rhythm sleep disorders include time-zone change (jet lag) syndrome, shift-work sleep disorder, irregular sleep/wake pattern, delayed sleep-phase syndrome, advanced sleep-phase syndrome, non-24-hour sleep/wake disorder, etc.

In certain embodiments, the invention contemplates the treatment of amnesia. Complaints of memory problems are common. Poor concentration, poor arousal and poor attention all may disrupt the memory process to a degree. The subjective complaint of memory problems therefore must be distinguished from true amnesias. This is usually done at the bedside in a more gross evaluation and through specific neuropsychological tests. Defects in visual and verbal memory can be separated through such tests. In amnesias there is by definition a preservation of other mental capacities such as logic. The neurobiologic theory of memory would predict that amnesias would have relatively few pathobiologic variations. Clinically the problem of amnesias often appears as a result of a sudden illness in an otherwise healthy person. Amnesias are described as specific defects in declarative memory. Faithful encoding of memory requires a registration, rehearsal, and retention of information. The first two elements appear to involve the hippocampus and medial temporal lobe structures. The retention or storage appears to involve the heteromodal association areas. Amnesia can be experienced as a loss of stored memory or an inability to form new memories. The loss of stored memories is known as retrograde amnesia. The inability to form new memories is known as anterograde amnesia.

Exemplary forms of amnesias which may be treated by the subject method include amnesias of short duration, alcoholic blackouts, Wernicke-Korsakoff's (early), partial complex seizures, transient global amnesia, those which are related to medication, such as triazolam (Halcion), and basilar artery migraines. The subject method may also be used to treat amnesias of longer duration, such as post concussive or as the result of Herpes simplex encephalitis.

The methods and compositions of the present invention can be used to treat or otherwise reduce the severity of any other CNS related condition. Such conditions may include, for example, learning disabilities, memory-loss conditions, eating disorders, or drug addiction (e.g., nicotine addiction). In certain embodiments, the CNS-related condition is not a neurodegenerative disease and/or a movement disorder.

The subject method can also be used to treat normal individuals for whom improved declarative memory is desired.

Certain embodiments of the invention relates to a method for treating any of the disorders described above, more specifically depression and ADHD (adult or child), comprising co-administering (e.g., simultaneously or at different times) to the subject an amount of a Hh agonist sufficient to treat the attention component of ADHD, and optionally an amount of a dopamine reuptake inhibitor sufficient to treat the movement disorder component. Activating the Hh pathway is expected to positively modulate appropriate neurogenesis and augment synaptic transmission, alleviating symptoms of ADHD that stems from deficient neuronal signaling. In certain embodiments, the Hh agonist and the dopamine reuptake inhibitor are administered simultaneously. In certain embodiments, the Hh agonist and the dopamine reuptake inhibitor are administered as part of a single composition. In certain embodiments, the composition is for oral administration or for transdermal administration.

Furthermore, one aspect of the present invention relates to the methods and compositions using a combination of a Hh agonist and a dopamine re-uptake inhibitor. A variety of dopamine transporter inhibitors (also called dopamine uptake inhibitors; herein referred to as active compounds) of diverse structure are known. See, e.g., S. Berger, U.S. Pat. No. 5,217,987; J. Boja et al. (1995) Molec. Pharmacol. 47: 779-786; C. Xu et al. (1995) Biochem. Pharmacol. 49:339-50; B. Madras et al. (1994) Eur. J. Pharmacol. 267: 167-73; F. Carroll et al. (1994) J. Med. Chem. 37: 2865-73; A. Eshleman et al. (1994) Molec. Pharmacol. 45: 312-16; R. Heikkila and L. Manzino (1984) Eur. J. Pharmacol. 103: 241-8. Dopamine transporter inhibitors are, in general, ligands that bind in a stereospecific manner to the dopamine transporter protein. Examples of such compounds are:
(1) tricyclic antidepressants such as buprion, nomifensine, and amineptin;
(2) 1,4-disubstituted piperazines, or piperazine analogs, such as 1-[2-[bis(4-fluorophenyl)methoxy]ethyl]-4-(3-phenylpropyl)piperazine dihydrochloride (or GBR12909), 1-[2-[bis(phenyl) methoxy]ethyl]-4-(3-phenylpropyl)piperazine dihydrochloride (for GBR12934), and GBR13069;
(3) tropane analogs, or (disubstituted phenyl) tropane-2 beta-carboxylic acid methyl esters, such as 3 [beta]-(4-fluorophenyl)tropane-2 [beta]-carboxylic acid methyl ester (or WIN 35,428) and 3 [beta]-(4-iodophenyl)tropane-2 [beta]-carboxylic acid isopropyl ester (RTI-121);
(4) substituted piperidines, or piperidine analogs, such as N-[1-(2-benzo[beta]-thiophenyl)cyclohexyl]piperidine, indatraline, and 4-[2-[bis(4-fluorophenyl)methoxy]ethyl]-1-(3-phenylpropyl)piperidine (or 0-526);
(5) quinoxaline derivatives, or quinoxaline analogs, such as 7-trifluoromethyl-4-(4-methyl-1-piperazinyl)pyrrolo[1,2-[alpha]]-quinoxaline (or CGS 12066b); and
(6) other compounds that are inhibitors of dopamine reuptake, such as mazindol, benztropine, bupropion, phencyclidine, methylphenidate, etc.

The methods of present invention may be carried out using various agents that stimulate the Hh signaling pathway. Such agents include Hh polypeptides and their functional equivalents, small bioactive molecules, antibodies, etc. The Hh agonists may be inhibitors or suppressors of the Hh signaling pathway. Examples of such inhibitor are RNAi constructs. These agents and compositions comprising them are described below in detail.

In certain preferred embodiments, the Hh agonists used to practice the methods of the present invention activate Hh-mediated signal transduction with an ED₅₀ of 1 mM or less, more preferably of 1 μM or less, and even more preferably of 1 nM or less.

In certain embodiments, the subject method can be carried out by administering RNAi modulators of suppressors of the Hh signaling pathway. The Hh signaling pathway comprises multiple regulatory elements, some of which, like patched, are identified as negative regulators. Inhibiting or antagonizing these negative regulators will result in activation of the Hh signaling pathway. In certain preferred embodiments, the subject Hh agonists can be chosen on the basis of their selectively for the Hh signaling pathway. This selectivity can be for the Hh signaling pathway versus other pathways, such as the wingless pathway which shares certain components with the Hh pathway; or for selectivity between particular Hh signaling pathways using one of several homologs, e.g., ptc-1 v. ptc-2, etc.

In other embodiments, the subject method can be carried out by administering a gene activation construct, wherein the gene activation construct is deigned to recombine with a genomic hh gene of the patient to provide a heterologous transcriptional regulatory sequence operatively linked to a coding sequence of the hh gene.

In still other embodiments, the subject method can be practiced with the administration of a gene therapy construct encoding a Hh polypeptide or its equivalent. For instance, the gene therapy construct can be provided in a composition selected from a recombinant viral particle, a liposome, and a poly-cationic nucleic acid binding agent. These gene therapy agents are described below.

In one embodiment of the methods described herein, the subject is any animal or artificially modified animal capable of becoming afflicted with the disorder. The subjects include but are not limited to a human being, a primate, an equine, an ovine, an avian, a bovine, a porcine, a canine, a feline or a murine subject. In a preferred embodiment, the subject is a human being.

In one embodiment of the methods described herein, the agent is administered by any of the following routes: intralesional, intraperitoneal, subcutaneous, intramuscular or intravenous injection; infusion; liposome-mediated delivery; topical, intrathecal, gingival pocket, rectal, intrabronchial, nasal, transmucosal, intestinal, oral, ocular or otic delivery. The compounds and/or agents of the subject invention may be delivered via a capsule which allows sustained release of the agent or the peptide over a period of time. Controlled or sustained release compositions include formulation in lipophilic depots (e.g., fatty acids, waxes, oils). Also encompassed by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In certain embodiments, a source of a Hh agonist is stereotactically provided within or proximate to the area of degeneration.

In one embodiment of the methods described herein, the effective amount of the agent is between about 1 mg and about 50 mg per kg body weight of the subject. In one embodiment, the effective amount of the agent is between about 2 mg and about 40 mg per kg body weight of the subject. In one embodiment, the effective amount of the agent is between about 3 mg and about 20 mg per kg body weight of the subject. However, it is understood by one skilled in the art that the dose of the composition of the invention will vary depending on the subject and upon the particular route of administration used. It is routine in the art to adjust the dosage to suit the individual subjects. Additionally, the effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective.

The compound may be delivered hourly, daily, weekly, monthly, yearly (e.g., in a time release form) or as a one time delivery. The delivery may be continuous delivery for a period of time, e.g., intravenous delivery. In one embodiment of the methods described herein, the agent is administered at least once per day. In one embodiment, the agent is administered daily. In one embodiment, the agent is administered every other day. In one embodiment, the agent is administered every 6 to 8 days. In one embodiment, the agent is administered weekly.

In certain embodiments, the methods include co-administration with the agent to stimulate the Hh signaling pathway, one or more a neuronal growth factor, a neuronal survival factor, or a neuronal trophic factor. Examples include nerve growth factor, ciliary neurotrophic growth factor, schwanoma-derived growth factor, glial growth factor, striatal-derived neuronotrophic factor, platelet-derived growth factor, and scatter factor (HGF-SF).

Polypeptides and Mutants

In one embodiment, the agent to stimulate the pathway is a Hh polypeptide or its functional equivalent. The term “functional equivalent” includes fragments, mutants, and muteins of the protein that exhibit similar biological and physiological function as the protein to which they are compared.

In certain embodiments, the Hh polypeptides are modified by chemical moiety to enhance their stability or to alter the solubility or affinity to certain environment. Such modification may be an addition of a lipophilic moiety or moieties at one or more internal sites of the mature, processed extracellular domain, and may or may not be also derivatized with lipophilic moieties at the N or C-terminal residues of the mature polypeptide. In other embodiments, the polypeptide is modified at the C-terminal residue with a hydrophobic moiety other than a sterol. In still other embodiments, the polypeptide is modified at the N-terminal residue with a cyclic (preferably polycyclic) lipophilic group. Various combinations of the above are also contemplated.

As discussed above, several distinct genes of the hh family are found in vertebrates. The amino acid sequences of exemplary vertebrate Hh polypeptides are described herein as SEQ ID Nos: 1-8. Also described is the single Drosophila Hh polypeptide. The corresponding nucleic acid sequences are also described. According to the appended sequence listing, (see also Table 1) a chicken Shh polypeptide is encoded by SEQ ID No: 1; a mouse Dhh polypeptide is encoded by SEQ ID No:2; a mouse Ihh polypeptide is encoded by SEQ ID No:3; a mouse Shh polypeptide is encoded by SEQ ID No:4 a zebrafish Shh polypeptide is encoded by SEQ ID No:5; a human Shh polypeptide is encoded by SEQ ID No:6; a human Ihh polypeptide is encoded by SEQ ID No:7; a human Dhh polypeptide is encoded by SEQ ID No. 8; and a zebrafish Thh is encoded by SEQ ID No. 9.

TABLE 1
Guide to hedgehog sequences in Sequence Listing

| | Nucleotide | Amino Acid |
|---|---|---|
| Chicken Shh | SEQ ID No. 1 | SEQ ID No. 10 |
| Mouse Dhh | SEQ ID No. 2 | SEQ ID No. 11 |
| Mouse Ihh | SEQ ID No. 3 | SEQ ID No. 12 |
| Mouse Shh | SEQ ID No. 4 | SEQ ID No. 13 |
| Zebrafish Shh | SEQ ID No. 5 | SEQ ID No. 14 |
| Human Shh | SEQ ID No. 6 | SEQ ID No. 15 |
| Human Ihh | SEQ ID No. 7 | SEQ ID No. 16 |
| Human Dhh | SEQ ID No. 8 | SEQ ID No. 17 |
| Zebrafish Thh | SEQ ID No. 9 | SEQ ID No. 18 |
| Drosophila hh | SEQ ID No. 19 | SEQ ID No. 20 |

Preferably, the agent to use in the inventive methods and compositions is a Hh polypeptide. More preferably, the agent is a sonic Hh polypeptide. In one embodiment, the agent is a fragment of a Hh polypeptide. More preferably, it is an N-terminal fragment containing a binding site to a receptor for a Hh polypeptide. Even more preferably, the fragment is a 19 kDa N-terminal fragment of a human Hh polypeptide. In another embodiment, the agent is a polypeptide which shares at least 60, 70, 80, 90% amino acid sequence homology with any of the Hh amino acid sequences depicted as SEQ ID NOs: 10 to 18. Homology can be assessed by any conventional analysis algorithm such as for example, the Pileup sequence analysis software (Program Manual for the Wisconsin Package, 1996).

The agent of the methods and composition of the present invention may be mutants of Hh polypeptides. “Mutants” of a protein comprise an altered amino acid sequence, for example by one or more amino acid deletions, substitutions or additions such that the mutant retains the ability to bind to a target of the unaltered protein. Conservative substitutions are more likely to yield mutants that are functionally equivalent to the original protein. Such conservative substitutions may ones within the following groups: (1) glycine and alanine; (2) valine, isoleucine, and leucine; (3) aspartic acid and glutamic acid; (4) asparagine and glutamine; (5)serine and threonine; (6) lysine and arginine; (7) phenylalanine and tyrosine. Such substitutions may also be homologous substitutions such as within the following groups: (a) glycine, alanine, valine, leucine, and isoleucine; (b) phenylalanine, tyrosine, and tryptophan; (c) lysine, arginine, and histidine; (d) aspartic acid, and glutamic acid; (e) asparagine and glutamine; (f) serine and threonine; (g) cysteine and methionine.

In another preferred embodiment, the invention features the use of a polypeptide that modulates, e.g., mimics or antagonizes, the biological activity of a Hh polypeptide, which is encoded by a nucleic acid that comprises all or a portion of the nucleotide sequence of the coding region of a gene identical or homologous to the nucleotide sequence designated by one of SEQ ID No:1, SEQ ID No:2, SEQ ID No:3, SEQ ID No:4, SEQ ID No:5, SEQ ID No:6, SEQ ID No:7, SEQ ID No:8, or SEQ ID No:9. Preferably, the nucleic acid comprises a Hh-encoding portion that hybridizes under stringent conditions to a coding portion of one or more of the nucleic acids designated by SEQ ID Nos: 1-9.

The term “equivalent” is understood to include nucleotide sequences encoding functionally equivalent Hh polypeptides or functionally equivalent peptides having an activity of a vertebrate Hh polypeptide such as described herein. Equivalent nucleotide sequences will include sequences that differ by one or more nucleotide substitutions, additions or deletions, such as allelic variants; and will, therefore, include sequences that differ from the nucleotide sequence of the vertebrate hh cDNAs shown in SEQ ID Nos: 1-9 due to the degeneracy of the genetic code. Equivalents will also include nucleotide sequences that hybridize under stringent conditions (i.e., equivalent to about 20-27° C. below the melting temperature (Tm) of the DNA duplex formed in about 1 M salt) to the nucleotide sequences represented in one or more of SEQ ID Nos: 1-9. In one embodiment, equivalents will further include nucleic acid sequences derived from and evolutionarily related to, a nucleotide sequences shown in any of SEQ ID Nos: 1-9.

In yet a further preferred embodiment, the nucleic acid encoding a Hh polypeptide useful in the present invention hybridizes under stringent conditions to a nucleic acid probe corresponding to at least 12 consecutive nucleotides of either sense or antisense sequence of one or more of SEQ ID Nos: 1-9; though preferably to at least 20 consecutive nucleotides; and more preferably to at least 40, 50 or 75 consecutive nucleotides of either sense or antisense sequence of one or more of SEQ ID Nos: 1-9.

Fusion Proteins

In one embodiment, the agent to practice the methods of the invention is a fusion protein which comprises all or a portion of a Hh polypeptide and any other peptide portion, such as a marker or another biologically active protein. Additional domains may be included in the subject fusion proteins of this invention. It is widely appreciated that fusion proteins can also facilitate the expression of proteins, and accordingly, can be used in the expression of the Hh polypeptides of the present invention. For example, the fusion proteins may include domains that facilitate their purification, e.g., “histidine tags” or a glutathione-5-transferase domain. They may include “epitope tags” encoding peptides recognized by known monoclonal antibodies for the detection of proteins within cells or the capture of proteins by antibodies in vitro. In a preferred embodiment, the recombinant Hh polypeptide is a fusion protein containing a domain which facilitates its purification, such as a Hh/GST fusion protein.

It may be necessary in some instances to introduce an unstructured polypeptide linker region between an analog peptide and other portions of the chimeric protein. The linker can facilitate enhanced flexibility of the fusion protein. The linker can also reduce steric hindrance between any two fragments of the fusion protein. The linker can also facilitate the appropriate folding of each fragment to occur. The linker can be of natural origin, such as a sequence determined to exist in random coil between two domains of a protein. An exemplary linker sequence is the linker found between the C-terminal and N-terminal domains of the RNA polymerase a subunit. Other examples of naturally occurring linkers include linkers found in the lcI and LexA proteins. Alternatively, the linker can be of synthetic origin. For instance, the sequence (Gly₄Ser)₃ can be used as a synthetic unstructured linker. Linkers of this type are described in Huston et al. (1988) Proc. Nat. Acad. Sci. USA 85:4879; and U.S. Pat. No. 5,091,513.

In some embodiments it is preferable that the design of a linker involve an arrangement of domains which requires the linker to span a relatively short distance, preferably less than about 10 Å. However, in certain embodiments, depending, e.g., upon the selected domains and the configuration, the linker may span a distance of up to about 50 Å.

Within the linker, the amino acid sequence may be varied based on the preferred characteristics of the linker as determined empirically or as revealed by modeling. For instance, in addition to a desired length, modeling studies may show that side groups of certain amino acids may interfere with the biological activity of the fusion protein. Considerations in choosing a linker include flexibility of the linker, charge of the linker, and presence of some amino acids of the linker in the naturally occurring subunits. The linker can also be designed such that residues in the linker contact DNA, thereby influencing binding affinity or specificity, or to interact with other proteins. For example, a linker may contain an amino acid sequence that is recognized by a protease so that the activity of the chimeric protein could be regulated by cleavage. In some cases, particularly when it is necessary to span a longer distance between subunits or when the domains must be held in a particular configuration, the linker may optionally contain an additional folded domain.

Preparation of the Proteins

The agents described herein may be made by any means known to one skilled in the art. For example, a protein may be made by recombinant expression from a nucleic acid, such as a plasmid or vector comprising the encoding nucleic acid, wherein the plasmid or vector is in a suitable host cell, i.e., a host-vector system for the production of the polypeptide of interest. A variety of expression systems, both prokaryotic and eukaryotic, are known to a person skilled in the art for protein and peptide production and are commercially available. A small polypeptide of less than 50 amino acid residues may be chemically synthesized using methods well known to one skilled in the art.

The various cells, cell lines and DNA sequences that can be used for mammalian cell expression of the single-chain constructs of the invention are well characterized in the art and are readily available. Particular details of the transfection, expression, and purification of recombinant proteins are well documented in the art and are understood by those having ordinary skill in the art. Further details on the various technical aspects of each of the steps used in recombinant production of foreign genes in mammalian cell expression systems can be found in a number of texts and laboratory manuals in the art, such as Ausubel et al., ed., Current Protocols in Molecular Biology, John Wiley & Sons, New York, (1989).

As is known in the art, Hh polypeptides can be produced by standard biological techniques. For example, a host cell transfected with a nucleic acid vector directing expression of a nucleotide sequence encoding the subject polypeptides can be cultured under appropriate conditions to allow expression of the peptide to occur. The Hh polypeptide may be secreted and isolated from a mixture of cells and medium containing the recombinant Hh polypeptide. Alternatively, the peptide may be retained cytoplasmically by removing the signal peptide sequence from the recombinant hh gene and the cells harvested, lysed and the protein isolated. A cell culture includes host cells, media and other byproducts. Suitable media for cell culture are well known in the art.

Recombinant hh genes can be produced by ligating nucleic acid encoding an Hh polypeptide, or a portion thereof, into a vector suitable for expression in either prokaryotic cells, eukaryotic cells, or both. Expression vectors for production of recombinant forms of the subject Hh polypeptides include plasmids and other vectors. For instance, suitable vectors for the expression of a Hh polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR₃₂₂ ori, and in S. cerevisiae due to the replication determinant of the yeast 2 micron plasmid. In addition, drug resistance markers such as ampicillin can be used. In an illustrative embodiment, an Hh polypeptide is produced recombinantly utilizing an expression vector generated by sub-cloning the coding sequence of one of the hh genes represented in SEQ ID Nos: 1-9 or 19.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR₃₂₂, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning: A Laboratory Manual, 2nd Ed., ed. by Sambrook, Fritsch and Maniatis (Cold Spring Harbor Laboratory Press: 1989) Chapters 16 and 17.

In some instances, it may be desirable to express the recombinant Hh polypeptide by the use of a baculovirus expression system. Examples of such baculovirus expression systems comprise insect host cells such as Sf9 cells and a baculovirus-derived vector, such as pVL-derived vectors (such as pVL1392, pVL1393 and pVL941), pAcUW-derived vectors (such as pAcUW1), and pBlueBac-derived vectors (such as the 13-gal containing pBlueBac III).

When it is desirable to express only a portion of a Hh polypeptide, such as a form lacking a portion of the N-terminus, i.e. a truncation mutant which lacks the signal peptide, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing Hh-derived polypeptides in a host which produces MAP (e.g., E. coli or CM89 or S. cerevisiae), or in vitro by use of purified MAP (e.g., procedure of Miller et al., supra).

Additionally, in order to tailor the properties of the protein or functional equivalent thereof, one skilled appreciates that alterations may be made at the nucleic acid level from known protein sequences, such as by adding, substituting, deleting or inserting one or more nucleotides. Site-directed mutagenesis is the method of preference that may be employed to make mutated proteins. There are many site-directed mutagenesis techniques known to those of skill in the art, including but not limited to oligonucleotide-directed mutagenesis using PCR, such as is described in Sambrook, or using commercially available kits.

After having determined which amino acid residues contribute to the receptor-binding domain, it is possible for the skilled artisan to design synthetic peptides having amino acid sequences that define a pre-selected receptor-binding motif. A computer program useful in designing potentially bioactive peptidomimetics is described in U.S. Pat. No. 5,331,573, the disclosure of which is incorporated by reference herein.

In addition to choosing a desirable amino acid sequence, the skilled artisan using standard molecular modeling software packages, can design specific peptides having, for example, additional cysteine amino acids located at pre-selected positions to facilitate cyclization of the peptide of interest. Oxidation of the additional cysteine residues results in cyclization of the peptide thereby constraining the peptide in a conformation that mimics the conformation of the corresponding amino acid sequence in the native protein. It is contemplated, that any standard covalent linkage, for example, disulfide bonds, typically used to cyclize synthetic peptides maybe useful in the practice of the instant invention. Alternative cyclization chemistries are discussed in International Application PCT/WO 95/01800, the disclosure of which is incorporated herein by reference.

Alternatively, analogs which are small peptides, usually up to 50 amino acids in length, may be synthesized using standard solid-phase peptide synthesis procedures, for example, procedures similar to those described in Merrifield (1963) J. Am. Chem. Soc., 85:2149. For example, during synthesis, N-α-protected amino acids having protected side chains are added stepwise to a growing polypeptide chain linked by its C-terminal end to an insoluble polymeric support, e.g., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. Commonly used N-α-protecting groups include Boc, which is acid labile, and Fmoc, which is base labile.

Details of appropriate chemistries, resins, protecting groups, protected amino acids and reagents are well known in the art and so are not discussed in detail herein. See, for example, Atherton et al. (1963) Solid Phase Peptide Synthesis: A Practical Approach (IRL Press,), and Bodanszky (1993) Peptide Chemistry, A Practical Textbook, 2nd Ed., Springer-Verlag, and Fields et al. (1990) Int. J. Peptide Protein Res. 35:161-214, the disclosures of which are incorporated herein by reference.

Protein Recovery and Purification

The recombinant Hh polypeptide can be isolated from cell culture medium, host cells, or both using techniques known in the art for purifying proteins including preparative HPLC, e.g., gel filtration, partition and/or ion exchange chromatography, ultrafiltration, electrophoresis, and immunoaffinity purification with antibodies specific for such peptide. The choice of appropriate matrices and buffers are well known in the art and so are not described in detail herein.

Purification of an expressed protein can be facilitated and carried out reliably by engineering into the expression vector a purification tag such as poly-Histidine addition to the desired protein or polypeptide. Poly(His)-Hh polypeptides can be easily purified by affinity chromatography using a Ni²⁺ metal resin. The poly(His) leader sequence can then be subsequently removed by treatment with enterokinase (e.g., see Hochuli et al. (1987) J. Chromatography 411:177; and Janknecht et al. PNAS 88:8972).

GST-fusion proteins can enable easy purification of the Hh polypeptide, as for example by the use of glutathione-derivatized matrices (see, for example, Current Protocols in Molecular Biology, eds. Ausubel et al. (N.Y.: John Wiley & Sons, 1991)).

Protein Modification

There are a wide range of lipophilic moieties with which Hh polypeptides can be derivatized. The term “lipophilic group”, in the context of being attached to a Hh polypeptide, refers to a group having high hydrocarbon content thereby giving the group high affinity to lipid phases. A lipophilic group can be, for example, a relatively long chain alkyl or cycloalkyl (preferably n-alkyl) group having approximately 7 to 30 carbons. The alkyl group may terminate with a hydroxy or primary amine “tail”. To further illustrate, lipophilic molecules include naturally occurring and synthetic aromatic and non-aromatic moieties such as fatty acids, esters and alcohols, other lipid molecules, cage structures such as adamantane and buckminsterfullerenes, and aromatic hydrocarbons such as benzene, perylene, phenanthrene, anthracene, naphthalene, pyrene, chrysene, and naphthacene. For additional details of modifications of Hh polypeptides, see U.S. application Ser. No. 09/579,680, the disclosure of which is incorporated herein by reference in its entirety.

Particularly useful as lipophilic molecules are alicyclic hydrocarbons, saturated and unsaturated fatty acids and other lipid and phospholipid moieties, waxes, cholesterol, isoprenoids, terpenes and polyalicyclic hydrocarbons including adamantane and buckminsterfullerenes, vitamins, polyethylene glycol or oligoethylene glycol, (C1-C18)-alkyl phosphate diesters, —O—CH2—CH(OH)—O—(C12-C18)-alkyl, and in particular conjugates with pyrene derivatives. The lipophilic moiety can be a lipophilic dye suitable for use in the invention include, but are not limited to, diphenylhexatriene, Nile Red, N-phenyl-1-naphthylamine, Prodan, Laurodan, Pyrene, Perylene, rhodamine, rhodamine B, tetramethylrhodamine, Texas Red, sulforhodamine, 1,1′-didodecyl-3,3,3′,3′tetramethylindocarbocyanine perchlorate, octadecyl rhodamine B and the BODIPY dyes available from Molecular Probes Inc.

Other exemplary lipophilic moieties include aliphatic carbonyl radical groups include 1- or 2-adamantylacetyl, 3-methyladamant-1-ylacetyl, 3-methyl-3-bromo-1-adamantylacetyl, 1-decalinacetyl, camphoracetyl, camphaneacetyl, noradamantylacetyl, norbornaneacetyl, bicyclo[2.2.2.]-oct-5-eneacetyl, 1-methoxybicyclo[2.2.2.]-oct-5-ene-2-carbonyl, cis-5-norbornene-endo-2,3-dicarbonyl, 5-norbornen-2-ylacetyl, (1R)-(−)-myrtentaneacetyl, 2-norbornaneacetyl, anti-3-oxo-tricyclo[2.2.1.0<2,6>]-heptane-7-carbonyl, decanoyl, dodecanoyl, dodecenoyl, tetradecadienoyl, decynoyl or dodecynoyl.

Derivatizing the Hh Polypeptide

The Hh polypeptide can be linked to the hydrophobic moiety in a number of ways including by chemical coupling means, or by genetic engineering.

There are a large number of chemical cross-linking agents that are known to those skilled in the art. For the present invention, the preferred cross-linking agents are heterobifunctional cross-linkers, which can be used to link the Hh polypeptide and hydrophobic moiety in a stepwise manner. Heterobifunctional cross-linkers provide the ability to design more specific coupling methods for conjugating to proteins, thereby reducing the occurrences of unwanted side reactions such as homo-protein polymers. A wide variety of heterobifunctional cross-linkers are known in the art. These include: succinimidyl 4-(N-maleimidomethyl) cyclohexane-1-carboxylate (SMCC), m-Maleimidobenzoyl-N-hydroxysuccinimide ester (MBS); N-succinimidyl(4-iodoacetyl)aminobenzoate (SIAB), succinimidyl 4-(p-maleimidophenyl) butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

In addition to the heterobifunctional cross-linkers, there exist a number of other cross-linking agents including homobifunctional and photoreactive cross-linkers. Disuccinimidyl suberate (DSS), bismaleimidohexane (BMH) and dimethylpimelimidate.2 HCl (DMP) are examples of useful homobifunctional cross-linking agents, and bis-[B-(4-azidosalicylamido)ethyl]disulfide (BASED) and N-succinimidyl-6(4′-azido-2′-nitrophenyl-amino)hexanoate (SANPAH) are examples of useful photoreactive cross-linkers for use in this invention. For a recent review of protein coupling techniques, see Means et al. (1990) Bioconjugate Chemistry 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product.

Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds.

The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules. For instance, SMPB has a span of 14.5 angstroms.

Preparing protein-protein conjugates using heterobifunctional reagents is a two-step process involving the amine reaction and the sulfhydryl reaction. For the first step, the amine reaction, the protein chosen should contain a primary amine. This can be lysine epsilon amines or a primary alpha amine found at the N-terminus of most proteins. The protein should not contain free sulfhydryl groups. In cases where both proteins to be conjugated contain free sulfhydryl groups, one protein can be modified so that all sulfhydryls are blocked using for instance, N-ethylmaleimide (see Partis et al. (1983) J. Pro. Chem. 2:263, incorporated by reference herein). Ellman's Reagent can be used to calculate the quantity of sulfhydryls in a particular protein (see for example Ellman et al. (1958) Arch. Biochem. Biophys. 74:443 and Riddles et al. (1979) Anal. Biochem. 94:75, incorporated by reference herein).

The reaction buffer should be free of extraneous amines and sulfhydryls. The pH of the reaction buffer should be 7.0-7.5. This pH range prevents maleimide groups from reacting with amines, preserving the maleimide group for the second reaction with sulfhydryls.

The NHS-ester containing cross-linkers have limited water solubility. They should be dissolved in a minimal amount of organic solvent (DMF or DMSO) before introducing the cross-linker into the reaction mixture. The cross-linker/solvent forms an emulsion which will allow the reaction to occur.

The sulfo-NHS ester analogs are more water soluble, and can be added directly to the reaction buffer. Buffers of high ionic strength should be avoided, as they have a tendency to “salt out” the sulfo-NHS esters. To avoid loss of reactivity due to hydrolysis, the cross-linker is added to the reaction mixture immediately after dissolving the protein solution.

The reactions can be more efficient in concentrated protein solutions. The more alkaline the pH of the reaction mixture, the faster the rate of reaction. The rate of hydrolysis of the NHS and sulfo-NHS esters will also increase with increasing pH. Higher temperatures will increase the reaction rates for both hydrolysis and acylation.

Once the reaction is completed, the first protein is now activated, with a sulfhydryl reactive moiety. The activated protein may be isolated from the reaction mixture by simple gel filtration or dialysis. To carry out the second step of the cross-linking, the sulfhydryl reaction, the lipophilic group chosen for reaction with maleimides, activated halogens, or pyridyl disulfides must contain a free sulfhydryl. Alternatively, a primary amine may be modified with to add a sulfhydryl

In all cases, the buffer should be degassed to prevent oxidation of sulffiydryl groups. EDTA may be added to chelate any oxidizing metals that may be present in the buffer. Buffers should be free of any sulfhydryl containing compounds.

Maleimides react specifically with —SH groups at slightly acidic to neutral pH ranges (6.5-7.5). A neutral pH is sufficient for reactions involving halogens and pyridyl disulfides. Under these conditions, maleimides generally react with —SH groups within a matter of minutes. Longer reaction times are required for halogens and pyridyl disulfides.

The first sulfhydryl reactive-protein prepared in the amine reaction step is mixed with the sulfhydryl-containing lipophilic group under the appropriate buffer conditions. The conjugates can be isolated from the reaction mixture by methods such as gel filtration or by dialysis.

Exemplary activated lipophilic moieties for conjugation include: N-(1-pyrene)maleimide; 2,5-dimethoxystilbene-4′-maleimide, eosin-5-maleimide; fluorescein-5-maleimide; N-(4-(6-dimethylamino-2-benzofuranyl)phenyl)maleimide; benzophenone-4-maleimide; 4-dimethylaminophenylazophenyl-4′-maleimide (DABMI), tetramethylrhodamine-5-maleimide, tetramethylrhodamine-6-maleimide, Rhodamine Red™ C2 maleimide, N-(5-aminopentyl)maleimide, trifluoroacetic acid salt, N-(2-aminoethyl)maleimide, trifluoroacetic acid salt, Oregon Green™ 488 maleimide, N-(2-((2-(((4-azido-2,3,5,6-tetrafluoro)benzoyl) amino)ethyl)dithio)ethyl)maleimide (TFPAM-SS 1), 2-(1-(3-dimethylaminopropyl)-indol-3-yl)-3-(indol-3-yl) maleimide (bisindolylmaleimide; GF 109203×), BODIPY® FL N-(2-aminoethyl)maleimide, N-(7-dimethylamino-4-methylcoumarin-3-yl)maleimide (DACM), Alexa™ 488 C5 maleimide, Alexa™ 594 C5 maleimide, sodium salt N-(1-pyrene)maleimide, 2,5-dimethoxystilbene-4′-maleimide, eosin-5-maleimide, fluorescein-5-maleimide, N-(4-(6-dimethylamino-2-benzofuranyl)phenyl)maleimide, benzophenone-4-maleimide, 4-dimethylaminophenylazophenyl-4′-maleimide, 1-(2-maleimidylethyl)-4-(5-(4-methoxyphenyl)oxazol-2-yl)pyridinium methanesulfonate, tetramethylrhodamine-5-maleimide, tetramethylrhodamine-6-maleimide, Rhodamine Red™ C2 maleimide, N-(5-aminopentyl)maleimide, N-(2-aminoethyl)maleimide, N-(2-((2-(((4-azido-2,3,5,6-tetrafluoro)benzoyl)amino)ethyl)dithio)ethyl)maleimide, 2-(1-(3-dimethylaminopropyl)-indol-3-yl)-3-(indol-3-yl) maleimide, N-(7-dimethylamino-4-methylcoumarin-3-yl)maleimide (DACM), 11H-Benzo[α]fluorene, Benzo[α]pyrene.

In one embodiment, the Hh polypeptide can be derivatized using pyrene maleimide, which can be purchased from Molecular Probes (Eugene, Oreg.), e.g., N-(1-pyrene)maleimide or 1-pyrenemethyl iodoacetate (PMIA ester). As illustrated in FIG. 1, the pyrene-derived Hh polypeptide had an activity profile indicating that it was nearly 2 orders of magnitude more active than the unmodified form of the protein.

For those embodiments wherein the hydrophobic moiety is a polypeptide, the modified Hh polypeptide of this invention can be constructed as a fusion protein, containing the Hh polypeptide and the hydrophobic moiety as one contiguous polypeptide chain.

In certain embodiments, the lipophilic moiety is an amphipathic polypeptide, such as magainin, cecropin, attacin, melittin, gramicidin S, alpha-toxin of Staphylococcus aureus, alamethicin or a synthetic amphipathic polypeptide. Fusogenic coat proteins from viral particles can also be a convenient source of amphipathic sequences for the subject Hh polypeptides

Small Molecules

In certain embodiments, the agent to stimulate the Hh signaling pathway is a small molecule agonist.

Compounds useful in such methods and compositions include those represented by general formula (I): 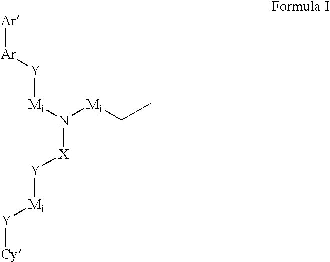
wherein, as valence and stability permit,
Ar and Ar′ independently represent substituted or unsubstituted aryl or heteroaryl rings;
Y, independently for each occurrence, is absent or represents —N(R)—, —O—, —S—, or —Se—;
X is selected from —C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;
R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;
Cy and Cy′ independently represent substituted or unsubstituted aryl, heterocyclyl, heteroaryl, or cycloalkyl, including polycyclic groups; and
i represents, independently for each occurrence, an integer from 0 to 5, preferably from 0 to 2.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Ar and Ar′ represent phenyl rings, e.g., unsubstituted or substituted with one or more groups including heteroatoms such as O, N, and S. In certain embodiments, at least one of Ar and Ar′ represents a phenyl ring. In certain embodiments, at least one of Ar and Ar′ represents a heteroaryl ring, e.g., a pyridyl, thiazolyl, thienyl, pyrimidyl, etc. In certain embodiments, Y and Ar′ are attached to Ar in a meta and/or 1,3-relationship.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuran, benzopyrrole, benzopyridine, etc. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, X is selected from —C(═O)—, —C(═S)—, and —S(O₂)—.

In certain embodiments, R represents H or lower alkyl, e.g., H or Me.

In certain embodiments, Cy represents a substituted or unsubstituted non-aromatic carbocyclic or heterocyclic ring, i.e., including at least one sp³ hybridized atom, and preferably a plurality of Sp hybridized atoms. In certain embodiments, Cy includes an amine within the atoms of the ring or on a substituent of the ring, e.g., Cy is pyridyl, imidazolyl, pyrrolyl, piperidyl, pyrrolidyl, piperazyl, etc., and/or bears an amino substituent. In certain embodiments, Cy is a 5- to 7-membered ring. In certain embodiments, Cy is directly attached to N. In embodiments wherein Cy is a six-membered ring directly attached to N and bears an amino substituent at the 4 position of the ring relative to N, the N and amine substituents may be disposed trans on the ring.

In certain embodiments, substituents on Ar or Ar′ are selected from halogen, lower alkyl, lower alkenyl, aryl, heteroaryl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein p and n, individually for each occurrence, represent integers from 0 to 10, preferably from 0 to 5.

In certain embodiments, compounds useful in the present invention may be represented by general formula (II): 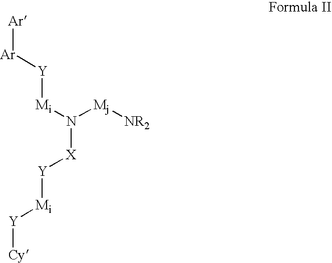
wherein, as valence and stability permit,
Ar and Ar′ independently represent substituted or unsubstituted aryl or heteroaryl rings;
Y, independently for each occurrence, is absent or represents —N(R)—, —O—, —S—, or —Se—;
X is selected from —C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne, wherein some or all occurrences of M in Mⱼ form all or part of a cyclic structure;
R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;
Cy′ represents a substituted or unsubstituted aryl, heterocyclyl, heteroaryl, or cycloalkyl, including polycyclic groups;
j represents, independently for each occurrence, an integer from 0 to 10, preferably from 2 to 7; and
i represents, independently for each occurrence, an integer from 0 to 5, preferably from 0 to 2.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Ar and Ar′ represent phenyl rings, e.g., unsubstituted or substituted with one or more groups including heteroatoms such as O, N, and S. In certain embodiments, at least one of Ar and Ar′ represents a phenyl ring. In certain embodiments, at least one of Ar and Ar′ represents a heteroaryl ring, e.g., a pyridyl, thiazolyl, thienyl, pyrimidyl, etc. In certain embodiments, Y and Ar′ are attached to Ar in a meta and/or 1,3-relationship.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N or NR₂.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuran, benzopyrrole, benzopyridine, etc. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, X is selected from —C(═O)—, —C(═S)—, and —S(O₂)—.

In certain embodiments, R represents H or lower alkyl, e.g., H or Me.

In certain embodiments, NR₂ represents a primary amine or a secondary or tertiary amine substituted with one or two lower alkyl groups, aryl groups, or aralkyl groups, respectively, preferably a primary amine or secondary amine.

In certain embodiments, substituents on Ar or Ar′ are selected from halogen, lower alkyl, lower alkenyl, aryl, heteroaryl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein p and n, individually for each occurrence, represent integers from 0 to 10, preferably from 0 to 5.

In certain embodiments, compounds useful in the present invention may be represented by general formula (III): 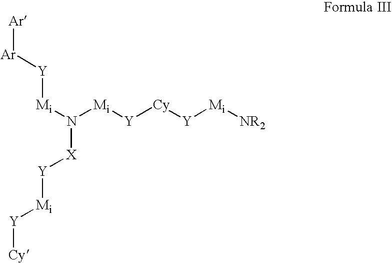
wherein, as valence and stability permit,
Ar and Ar′ independently represent substituted or unsubstituted aryl or heteroaryl rings;
Y, independently for each occurrence, is absent or represents —N(R)—, —O—, —S—, or —Se—;
X is selected from —C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;
R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;
Cy and Cy′ independently represent substituted or unsubstituted aryl, heterocyclyl, heteroaryl, or cycloalkyl, including polycyclic groups; and
i represents, independently for each occurrence, an integer from 0 to 5, preferably from 0 to 2.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Ar and Ar′ represent phenyl rings, e.g., unsubstituted or substituted with one or more groups including heteroatoms such as O, N, and S. In certain embodiments, at least one of Ar and Ar′ represents a phenyl ring. In certain embodiments, at least one of Ar and Ar′ represents a heteroaryl ring, e.g., a pyridyl, thiazolyl, thienyl, pyrimidyl, etc. In certain embodiments, Y and Ar′ are attached to Ar in a meta and/or 1,3-relationship.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N or NR₂.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuran, benzopyrrole, benzopyridine, etc. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, X is selected from —C(═O)—, —C(═S)—, and —S(O₂)—.

In certain embodiments, R represents H or lower alkyl, e.g., H or Me.

In certain embodiments, NR₂ represents a primary amine or a secondary or tertiary amine substituted with one or two lower alkyl groups, aryl groups, or aralkyl groups, respectively, preferably a primary amine or a secondary amine.

In certain embodiments, Cy represents a substituted or unsubstituted non-aromatic carbocyclic or heterocyclic ring, i.e., including at least one sp³ hybridized atom, and preferably a plurality of sp hybridized atoms. In certain embodiments, Cy is directly attached to N and/or to NR₂. In certain embodiments, Cy is a 5- to 7-membered ring. In embodiments wherein Cy is a six-membered ring directly attached to N and bears an amino substituent at the 4 position of the ring relative to N, the N and amine substituents may be disposed trans on the ring.

In certain embodiments, substituents on Ar or Ar′ are selected from halogen, lower alkyl, lower alkenyl, aryl, heteroaryl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein n and p, individually for each occurrence, represent integers from 0 to 10, preferably from 0 to 5.

In certain embodiments, compounds useful in the subject methods include compounds represented by general formula (IV): 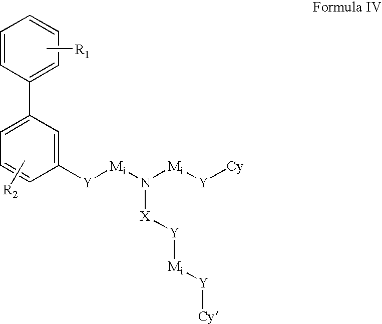
wherein, as valence and stability permit,
Cy′ represents a substituted or unsubstituted aryl or heteroaryl ring, including polycyclics;
Y, independently for each occurrence, is absent or represents —N(R)—, —O—, —S—, or —Se—;
X is selected from —C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;
R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;
R₁ and R₂ represent, independently and as valency permits, from 0-5 substituents on the ring to which it is attached, selected from halogen, lower alkyl, lower alkenyl, aryl, heteroaryl, carbonyl, thiocarbonyl, ketone, aldehyde, amino, acylamino, amido, amidino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above;
Cy represents substituted or unsubstituted aryl, heterocyclyl, heteroaryl, or cycloalkyl, including polycyclic groups;
i represents, independently for each occurrence, an integer from 0 to 5, preferably from 0 to 2; and
p and n, individually for each occurrence, represent integers from 0 to 10, preferably from 0 to 5.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Cy′ represents a substituted or unsubstituted bicyclic or heterocyclic ring system, preferably both bicyclic and heteroaryl, such as benzothiophene, benzofuran, benzopyrrole, benzopyridine, etc. In certain embodiments, Cy′ is directly attached to X. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N.

In certain embodiments, X is selected from —C(═O)—, —C(═S)—, and —S(O₂)—.

In certain embodiments, R represents H or lower alkyl, e.g., H or Me.

In certain embodiments, Cy represents a substituted or unsubstituted non-aromatic carbocyclic or heterocyclic ring, i.e., including at least one sp³ hybridized atom, and preferably a plurality of Sp hybridized atoms. In certain embodiments, Cy includes an amine within the atoms of the ring or on a substituent of the ring, e.g., Cy is pyridyl, imidazolyl, pyrrolyl, piperidyl, pyrrolidyl, piperazyl, etc., and/or bears an amino substituent. In certain embodiments, Cy is directly attached to N. In certain embodiments, Cy is a 5- to 7-membered ring. In embodiments wherein Cy is a six-membered ring directly attached to N and bears an amino substituent at the 4 position of the ring relative to N, the N and amine substituents may be disposed trans on the ring.

In certain embodiments, R₁ and R₂ represent, independently and as valency permits, from 0-5 substituents on the ring to which it is attached, selected from halogen, lower alkyl, lower alkenyl, carbonyl, thiocarbonyl, ketone, aldehyde, amino, acylamino, cyano, nitro, hydroxyl, sulfonyl, sulfoxido, sulfate, sulfonate, sulfamoyl, sulfonamido, —(CH₂)palkyl, —(CH₂)palkenyl, —(CH₂)palkynyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above.

In certain embodiments, compounds useful in the present invention may be represented by general formula (V): 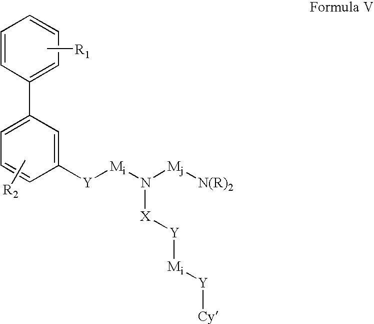
wherein, as valence and stability permit,
Cy′ represents a substituted or unsubstituted aryl or heteroaryl ring, including polycyclics;
Y, independently for each occurrence, is absent or represents —N(R)—, —O—, —S—, or —Se—;
X is selected from —C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;
R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;
R₁ and R₂ represent, independently and as valency permits, from 0-5 substituents on the ring to which it is attached, selected from halogen, lower alkyl, lower alkenyl, aryl, heteroaryl, carbonyl, thiocarbonyl, ketone, aldehyde, amino, acylamino, amido, amidino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above;
Cy′ represents a substituted or unsubstituted aryl, heterocyclyl, heteroaryl, or cycloalkyl, including polycyclic groups;
j represents, independently for each occurrence, an integer from 0 to 10, preferably from 2 to 7;
i represents, independently for each occurrence, an integer from 0 to 5, preferably from 0 to 2; and
p and n, individually for each occurrence, represent integers from 0 to 10, preferably from 0 to 5.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Cy′ represents a substituted or unsubstituted bicyclic or heterocyclic ring system, preferably both bicyclic and heteroaryl, such as benzothiophene, benzofuran, benzopyrrole, benzopyridine, etc. In certain embodiments, Cy′ is directly attached to X. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N or NR₂.

In certain embodiments, X is selected from —C(═O)—, —C(═S)—, and —S(O₂)—.

In certain embodiments, NR₂ represents a primary amine or a secondary or tertiary amine substituted with one or two lower alkyl groups, aryl groups, or aralkyl groups, respectively, preferably a primary or secondary amine.

In certain embodiments, R represents H or lower alkyl, e.g., H or Me.

In certain embodiments, R₁ and R₂ represent, independently and as valency permits, from 0-5 substituents on the ring to which it is attached, selected from halogen, lower alkyl, lower alkenyl, carbonyl, thiocarbonyl, ketone, aldehyde, amino, acylamino, cyano, nitro, hydroxyl, sulfonyl, sulfoxido, sulfate, sulfonate, sulfamoyl, sulfonamido, —(CH₂)palkyl, —(CH₂)palkenyl, —(CH₂)palkynyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above.

In certain embodiments, compounds useful in the present invention may be represented by general formula (VI): 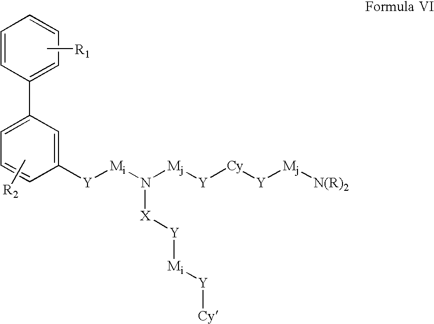
wherein, as valence and stability permit,
Cy′ represents a substituted or unsubstituted aryl or heteroaryl ring, including polycyclics;
Y, independently for each occurrence, is absent or represents —N(R)—, —O—, —S—, or —Se—;
X is selected from —C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;
R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;
Cy represents substituted or unsubstituted aryl, heterocyclyl, heteroaryl, or cycloalkyl, including polycyclic groups;
R₁ and R₂ represent, independently and as valency permits, from 0-5 substituents on the ring to which it is attached, selected from halogen, lower alkyl, lower alkenyl, aryl, heteroaryl, carbonyl, thiocarbonyl, ketone, aldehyde, amino, acylamino, amido, amidino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above;
i represents, independently for each occurrence, an integer from 0 to 5, preferably from 0 to 2; and
n and p, individually for each occurrence, represent integers from 0 to 10, preferably from 0 to 5.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Cy′ represents a substituted or unsubstituted bicyclic or heteroaryl ring system, preferably both bicyclic and heteroaryl, e.g., benzothiophene, benzofuran, benzopyrrole, benzopyridyl, etc. In certain embodiments, Cy′ is directly attached to X. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N or NR₂.

In certain embodiments, X is selected from —C(═O)—, —C(═S)—, and —S(O₂)—.

In certain embodiments, NR₂ represents a primary amine or a secondary or tertiary amine substituted with one or two lower alkyl groups, aryl groups, or aralkyl groups, respectively, preferably a primary amine.

In certain embodiments, R represents H or lower alkyl, e.g., H or Me.

In certain embodiments, Cy represents a substituted or unsubstituted non-aromatic carbocyclic or heterocyclic ring, i.e., including at least one sp³ hybridized atom, and preferably a plurality of sp hybridized atoms. In certain embodiments, Cy is directly attached to N and/or to NR₂. In certain embodiments, Cy is a 5- to 7-membered ring. In embodiments wherein Cy is a six-membered ring directly attached to N and bears an amino substituent at the 4 position of the ring relative to N, the N and amine substituents may be disposed trans on the ring.

In certain embodiments, R₁ and R₂ represent, independently and as valency permits, from 0-5 substituents on the ring to which it is attached, selected from halogen, lower alkyl, lower alkenyl, carbonyl, thiocarbonyl, ketone, aldehyde, amino, acylamino, cyano, nitro, hydroxyl, sulfonyl, sulfoxido, sulfate, sulfonate, sulfamoyl, sulfonamido, —(CH₂)palkyl, —(CH₂)palkenyl, —(CH₂)palkynyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above.

In certain embodiments, a subject compound has the structure of Formula VII: 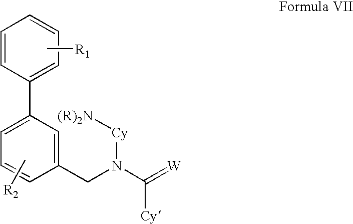
wherein, as valence and stability permit,
Cy represents a substituted or unsubstituted heterocyclyl or cycloalkyl;
Cy′ is a substituted or unsubstituted aryl or heteroaryl ring, including polycyclics;
W is O or S;
R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;
R₁ and R₂ represent, independently and as valency permits, from 0-5 substituents on the ring to which it is attached, selected from halogen, lower alkyl, lower alkenyl, aryl, heteroaryl, carbonyl, thiocarbonyl, ketone, aldehyde, amino, acylamino, amido, amidino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above;
n and p, individually for each occurrence, represent integers from 0 to 10, preferably from 0 to 5.

In certain embodiments, Cy′ represents a substituted or unsubstituted bicyclic or heteroaryl ring system, preferably both bicyclic and heteroaryl, e.g., benzothiophene, benzofuran, benzopyrrole, benzopyridyl, etc. In certain other embodiments, Cy′ represents an aryl or heteroaryl ring substituted at least with a substituted or unsubstituted aryl or heteroaryl ring, i.e., to form a biaryl ring system.

In certain embodiments, NR₂ represents a primary amine or a secondary or tertiary amine substituted with one or two lower alkyl groups, aryl groups, or aralkyl groups, respectively, preferably a primary or secondary amine.

In certain embodiments, Cy represents a substituted or unsubstituted saturated carbocyclic or heterocyclic ring, i.e., composed of a plurality of sp³ hybridized atoms. In certain embodiments, Cy is a 5- to 7-membered ring. In embodiments wherein Cy is a six-membered ring directly attached to N and bears an amino substituent at the 4 position of the ring relative to N, the N and amine substituents may be disposed trans on the ring.

In certain embodiments, R₁ and R₂ represent, independently and as valency permits, from 0-5 substituents on the ring to which it is attached, selected from halogen, lower alkyl, lower alkenyl, carbonyl, thiocarbonyl, ketone, aldehyde, amino, acylamino, cyano, nitro, hydroxyl, sulfonyl, sulfoxido, sulfate, sulfonate, sulfamoyl, sulfonamido, —(CH₂)palkyl, —(CH₂)palkenyl, —(CH₂)palkynyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above.

In certain embodiments, a subject compound has a structure of Formula VIII: 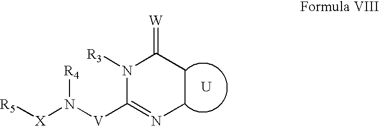
wherein, as valence and stability permit,
U represents a substituted or unsubstituted aryl or heteroaryl ring fused to the nitrogen-containing ring;
V represents a lower alkylene group, such as methylene, 1,2-ethylene, 1,1-ethylene, 1,1-propylene, 1,2-propylene, 1,3-propylene, etc.;
W represents S or O, preferably 0;
X represents C═O, C═S, or SO₂;
R₃ represents substituted or unsubstituted aryl, heteroaryl, lower alkyl, lower alkenyl, lower alkynyl, carbocyclyl, carbocyclylalkyl, heterocyclyl, heterocyclylalkyl, aralkyl, or heteroaralkyl;
R₄ represents substituted or unsubstituted aralkyl or lower alkyl, such as phenethyl, benzyl, or aminoalkyl, etc.;
R₅ represents substituted or unsubstituted aryl, heteroaryl, aralkyl, or heteroaralkyl, including polycyclic aromatic or heteroaromatic groups.

In certain embodiments, U represents a phenyl ring fused to the nitrogen-containing ring.

In certain embodiments, R₃ is selected from substituted or unsubstituted aryl, heteroaryl, lower alkyl, lower alkenyl, aralkyl, and heteroaralkyl.

In certain embodiments, R₄ is an unsubstituted lower alkyl group, or is a lower alkyl group substituted with a secondary or tertiary amine.

In certain embodiments, R₅ is selected from substituted or unsubstituted phenyl or naphthyl, or is a diarylalkyl group, such as 2,2-diphenylethyl, diphenylmethyl, etc.

In certain embodiments, subject compounds include compounds represented by general formula (IX): 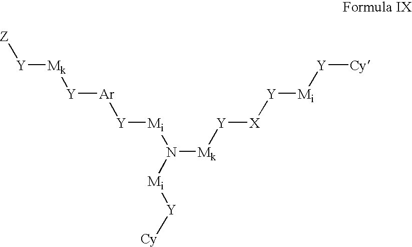
wherein, as valence and stability permit,
Ar represents a substituted or unsubstituted aryl or heteroaryl ring;
Z is absent or represents a substituted or unsubstituted aryl, carbocyclyl, heterocyclyl, or heteroaryl ring, or a lower alkyl, nitro, cyano, or halogen substituent;
Y, independently for each occurrence, is absent or represents —N(R)—, —O—, —S—, or —Se—, provided that if Z is not a ring, then Y attached to Z is absent;
X is selected from —C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;
R represents, independently for each occurrence, H or substituted or unsubstituted aryl, heterocyclyl, carbocyclyl, heteroaryl, aralkyl, heteroaralkyl, heterocyclylalkyl, carbocyclylalkyl, alkynyl, alkenyl, or alkyl, or two R taken together may form a 4- to 8-membered ring, e.g., with N;
Cy and Cy′ independently represent substituted or unsubstituted aryl, heterocyclyl, heteroaryl, or cycloalkyl, including polycyclic groups;
i represents, independently for each occurrence, an integer from 0 to 5, preferably from 0 to 2; and
k represents an integer from 0 to 3, preferably from 0 to 2.

In certain embodiments, M represents, independently for each occurrence, a substituted or unsubstituted methylene group, such as —CH₂—, —CHF—, —CHOH—, —CH(Me)—, —C(═O)—, etc. In certain embodiments, i represents 0 for all occurrences except in the sequence N-Mᵢ-Y—Ar, where i represents 1.

In certain embodiments, Ar and X independently represent substituted or unsubstituted aryl or heteroaryl rings, e.g., unsubstituted or substituted with one or more groups optionally including heteroatoms such as O, N, and S. In certain embodiments, Ar represents a phenyl ring. In certain embodiments, at least one of Ar represents a heteroaryl ring, e.g., a pyridyl, thiazolyl, thienyl, pyrimidyl, furanyl, etc. In certain embodiments, the occurrences of Y attached to Ar are disposed in a meta and/or 1,3-relationship.

In certain embodiments, Y is absent from all positions. In certain embodiments, the only present occurrence of Y is attached to Mk. In embodiments wherein Y is present in a position, i or k preferably represents 2 in an adjacent M_{i/k} if i/k=0 would result in two occurrences of Y being directly attached to each other, or an occurrence of Y being directly attached to N. In certain embodiments, where two occurrences of Y are attached to M, at least one such occurrence of Y is absent. In certain embodiments, no more than two occurrences of Y are present.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuran, benzopyrrole, benzopyridine, etc. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system. In certain embodiments, Cy′ represents a benzo(b)thien-2-yl, preferably a 3-chloro-benzo(b)thien-2-yl, 3-fluoro-benzo(b)thien-2-yl, or 3-methyl-benzo(b)thien-2-yl, e.g., wherein the benzo ring is substituted with from 1-4 substituents selected from halogen, nitro, cyano, methyl (e.g., including halomethyl, such as CHCl₂ and CF₃), and ethyl (e.g., including haloethyl, such as CH₂CCl₃, C₂F₅, etc.), preferably from halogen and methyl (e.g., including halomethyl, such as CHCl₂ and CF₃). In certain such embodiments Cy′ represents a 3-chloro-benzo(b)thien-2-yl, 3-fluoro-benzo(b)thien-2-yl, or 3-methyl-benzo(b)thien-2-yl wherein the benzo ring is substituted with fluoro at the 4-position (peri to the 3-substituent on the thienyl ring) and, optionally, at the 7-position (‘peri’ to the S of the thienyl ring).

In certain embodiments, X is selected from —C(═O)—, —C(═S)—, and —S(O₂)—.

In certain embodiments, Cy represents a substituted or unsubstituted non-aromatic carbocyclic or heterocyclic ring, i.e., including at least one sp³ hybridized atom, and preferably a plurality of sp³ hybridized atoms. In certain embodiments, Cy includes an amine within the atoms of the ring or on a substituent of the ring, e.g., Cy is pyridyl, imidazolyl, pyrrolyl, piperidyl, pyrrolidyl, piperazyl, etc., and/or bears an amino substituent. In certain embodiments, Cy is a 5- to 7-membered ring. In certain embodiments, Cy is directly attached to N. In embodiments wherein Cy is a six-membered ring directly attached to N and bears an amino substituent at the 4 position of the ring relative to N, the N and amine substituents may be disposed trans on the ring.

In certain embodiments, substituents on Ar or Z, where Z is an aryl or heteroaryl ring, are selected from halogen, lower alkyl, lower alkenyl, aryl, heteroaryl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein n and p, individually for each occurrence, represent integers from 0 to 10, preferably from 0 to 5.

In certain embodiments, Z is directly attached to Ar, or attached to Ar through a chain of one or two atoms. In certain embodiments, Z-Y-M, taken together, is absent.

In certain embodiments, compounds useful in the present invention may be represented by general formula (X): 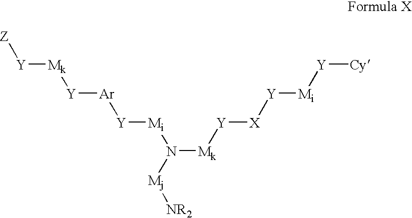
wherein, as valence and stability permit,
Ar represents a substituted or unsubstituted aryl or heteroaryl ring;
Z is absent or represents a substituted or unsubstituted aryl, carbocyclyl, heterocyclyl, or heteroaryl ring, or a lower alkyl, nitro, cyano, or halogen substituent;
Y, independently for each occurrence, is absent or represents —N(R)—, —O—, —S—, or —Se—, provided that if Z is not a ring, then Y attached to Z is absent;
X is selected from —C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
R represents, independently for each occurrence, H or substituted or unsubstituted aryl, heterocyclyl, carbocyclyl, heteroaryl, aralkyl, heteroaralkyl, heterocyclylalkyl, carbocyclylalkyl, alkynyl, alkenyl, or alkyl, or two R taken together may form a 4- to 8-membered ring, e.g., with N;
Cy′ represents a substituted or unsubstituted aryl, heterocyclyl, heteroaryl, or cycloalkyl, including polycyclic groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne, wherein some or all occurrences of M in Mⱼ form all or part of a cyclic structure;
j represents, independently for each occurrence, an integer from 2 to 10, preferably from 2 to 7;
i represents, independently for each occurrence, an integer from 0 to 5, preferably from 0 to 2; and
k represents an integer from 0 to 3, preferably from 0 to 2.

In certain embodiments, NR₂ represents a primary amine or a secondary or tertiary amine substituted with one or two lower alkyl groups, aryl groups, or aralkyl groups, respectively, preferably a primary or secondary amine.

In certain embodiments, M represents, independently for each occurrence, a substituted or unsubstituted methylene group, such as —CH₂—, —CHF—, —CHOH—, —CH(Me)—, —C(═O)—, etc. In certain embodiments, i represents 0 for all occurrences except in the sequence N-Mᵢ-Y—Ar, where i represents 1.

In certain embodiments, Ar and X independently represent substituted or unsubstituted aryl or heteroaryl rings, e.g., unsubstituted or substituted with one or more groups optionally including heteroatoms such as O, N, and S. In certain embodiments, Ar represents a phenyl ring. In certain embodiments, Ar represents a heteroaryl ring, e.g., a pyridyl, thiazolyl, thienyl, pyrimidyl, furanyl, etc. In certain embodiments, the occurrences of Y attached to Ar are disposed in a meta and/or 1,3-relationship.

In certain embodiments, Y is absent from all positions. In certain embodiments, the only present occurrence of Y is attached to Mₖ. In embodiments wherein Y is present in a position, i or k preferably represents 2 in an adjacent M_{i/k} if i/k=0 would result in two occurrences of Y being directly attached to each other, or an occurrence of Y being directly attached to N. In certain embodiments, where two occurrences of Y are attached to M, at least one such occurrence of Y is absent. In certain embodiments, no more than two occurrences of Y are present.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuran, benzopyrrole, benzopyridine, etc. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system. In certain embodiments, Cy′ represents a benzo(b)thien-2-yl, preferably a 3-chloro-benzo(b)thien-2-yl, 3-fluoro-benzo(b)thien-2-yl, or 3-methyl-benzo(b)thien-2-yl, e.g., wherein the benzo ring is substituted with from 1-4 substituents selected from halogen, nitro, cyano, methyl (e.g., including halomethyl, such as CHCl₂ and CF₃), and ethyl (e.g., including haloethyl, such as CH₂CCl₃, C₂F₅, etc.), preferably from halogen and methyl (e.g., including halomethyl, such as CHCl₂ and CF₃). In certain such embodiments Cy′ represents a 3-chloro-benzo(b)thien-2-yl, 3-fluoro-benzo(b)thien-2-yl, or 3-methyl-benzo(b)thien-2-yl wherein the benzo ring is substituted with fluoro at the 4-position (peri to the 3-substituent on the thienyl ring) and, optionally, at the 7-position (‘peri’ to the S of the thienyl ring).

In certain embodiments, X is selected from —C(═O)—, —C(═S)—, and —S(O₂)—.

In certain embodiments, substituents on Ar or Z, where Z is an aryl or heteroaryl ring, are selected from halogen, lower alkyl, lower alkenyl, aryl, heteroaryl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚ-lower alkyl, —(CH₂)ₚNR-lower alkenyl, —NR(CH₂)ₙR, and protected forms of the above, wherein n and p, individually for each occurrence, represent integers from 0 to 10, preferably from 0 to 5.

In certain embodiments, Z is directly attached to Ar, or attached to Ar through a chain of one or two atoms. In certain embodiments, Z-Y-M, taken together, is absent.

In certain embodiments, compounds useful in the present invention may be represented by general formula (XI): 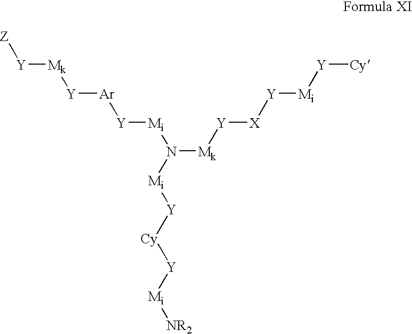
wherein, as valence and stability permit,
Ar represents a substituted or unsubstituted aryl or heteroaryl ring;
Z is absent or represents a substituted or unsubstituted aryl, carbocyclyl, heterocyclyl, or heteroaryl ring, or a lower alkyl, nitro, cyano, or halogen substituent;
Y, independently for each occurrence, is absent or represents —N(R)—, —O—, —S—, or —Se—, provided that if Z is not a ring, then Y attached to Z is absent;
X is selected from —C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;
R represents, independently for each occurrence, H or substituted or unsubstituted aryl, heterocyclyl, carbocyclyl, heteroaryl, aralkyl, heteroaralkyl, heterocyclylalkyl, carbocyclylalkyl, alkynyl, alkenyl, or alkyl, or two R taken together may form a 4- to 8-membered ring, e.g., with N;
Cy and Cy′ independently represent substituted or unsubstituted aryl, heterocyclyl, heteroaryl, or cycloalkyl, including polycyclic groups;
i represents, independently for each occurrence, an integer from 0 to 5, preferably from 0 to 2; and
k represents an integer from 0 to 3, preferably from 0 to 2.

In certain embodiments, NR₂ represents a primary amine or a secondary or tertiary amine substituted with one or two lower alkyl groups, aryl groups, or aralkyl groups, respectively, preferably a primary or secondary amine.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Ar and Z independently represent substituted or unsubstituted aryl or heteroaryl rings, e.g., unsubstituted or substituted with one or more groups optionally including heteroatoms such as O, N, and S. In certain embodiments, at least one of Ar and Z represents a phenyl ring. In certain embodiments, at least one of Ar and Z represents a heteroaryl ring, e.g., a pyridyl, thiazolyl, thienyl, pyrimidyl, furanyl, etc. In certain embodiments, the occurrences of Y attached to Ar are disposed in a meta and/or 1,3-relationship.

In certain embodiments, Y is absent from all positions. In certain embodiments, the only present occurrence of Y is attached to Mₖ. In embodiments wherein Y is present in a position, i or k preferably represents 2 in an adjacent M_{i/k} if i/k=0 would result in two occurrences of Y being directly attached to each other, or an occurrence of Y being directly attached to N. In certain embodiments, where two occurrences of Y are attached to M, at least one such occurrence of Y is absent. In certain embodiments, no more than two occurrences of Y are present.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuran, benzopyrrole, benzopyridine, etc. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system. In certain embodiments, Cy′ represents a benzo(b)thien-2-yl, preferably a 3-chloro-benzo(b)thien-2-yl, 3-fluoro-benzo(b)thien-2-yl, or 3-methyl-benzo(b)thien-2-yl, e.g., wherein the benzo ring is substituted with from 1-4 substituents selected from halogen, nitro, cyano, methyl (e.g., including halomethyl, such as CHCl₂ and CF₃), and ethyl (e.g., including haloethyl, such as CH₂CCl₃, C₂F₅, etc.), preferably from halogen and methyl (e.g., including halomethyl, such as CHCl₂ and CF₃). In certain such embodiments Cy′ represents a 3-chloro-benzo(b)thien-2-yl, 3-fluoro-benzo(b)thien-2-yl, or 3-methyl-benzo(b)thien-2-yl wherein the benzo ring is substituted with fluoro at the 4-position (peri to the 3-substituent on the thienyl ring) and, optionally, at the 7-position (‘peri’ to the S of the thienyl ring).

In certain embodiments, Cy represents a substituted or unsubstituted non-aromatic carbocyclic or heterocyclic ring, i.e., including at least one sp³ hybridized atom, and preferably a plurality of sp³ hybridized atoms. In certain embodiments, Cy is a 5- to 7-membered ring. In certain embodiments, Cy is directly attached to N and/or to NR₂. In embodiments wherein Cy is a six-membered ring directly attached to N and bears an amino substituent at the 4 position of the ring relative to N, the N and amine substituents may be disposed trans on the ring.

In certain embodiments, X is selected from —C(═O)—, —C(═S)—, and —S(O₂)—.

In certain embodiments, substituents on Ar or Z, where Z is an aryl or heteroaryl ring, are selected from halogen, lower alkyl, lower alkenyl, aryl, heteroaryl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein n and p, individually for each occurrence, represent integers from 0 to 10, preferably from 0 to 5.

In certain embodiments, Z is directly attached to Ar, or attached to Ar through a chain of one or two atoms. In certain embodiments, Z-Y-M, taken together, is absent.

In certain embodiments, compounds useful in the present invention may be represented by general formula (XII): 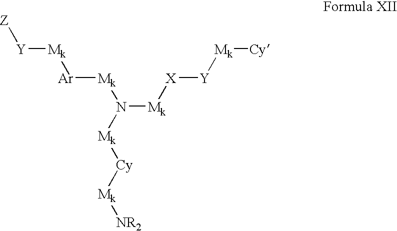
wherein, as valence and stability permit,
Ar represents a substituted or unsubstituted aryl or heteroaryl ring;
Z is absent or represents a substituted or unsubstituted aryl, carbocyclyl, heterocyclyl, or heteroaryl ring, or a lower alkyl, nitro, cyano, or halogen substituent;
Y, independently for each occurrence, is absent or represents —N(R)—, —O—, —S—, or —Se—, provided that if Z is not a ring, then Y attached to Z is absent;
X is selected from —C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;
R, independently for each occurrence, represents H or substituted or unsubstituted aryl, heterocyclyl, carbocyclyl, heteroaryl, aralkyl, heteroaralkyl, heterocyclylalkyl, carbocyclylalkyl, alkynyl, alkenyl, or alkyl;
Cy and Cy′ independently represent substituted or unsubstituted aryl, heterocyclyl, heteroaryl, or cycloalkyl, including polycyclic groups; and
k represents an integer from 0 to 1.

In certain embodiments, NR₂ represents a primary amine or a secondary or tertiary amine substituted with one or two lower alkyl groups, respectively, preferably a primary or secondary amine, most preferably a secondary amine.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Y is absent from all positions. In certain embodiments, where Y is adjacent to Mk, either Y is absent or k=0. In certain embodiments, for at least one occurrence of Mₖ attached to Cy, k=0, optionally for both occurrences. In certain embodiments, for Mₖ attached to Ar and N, k=1.

In certain embodiments, Ar and Z independently represent substituted or unsubstituted aryl or heteroaryl rings, e.g., unsubstituted or substituted with one or more groups optionally including heteroatoms such as O, N, and S. In certain embodiments, at least one of Ar and Z represents a phenyl ring. In certain embodiments, at least one of Ar and Z represents a heteroaryl ring, e.g., a pyridyl, thiazolyl, thienyl, pyrimidyl, furanyl, etc. In certain embodiments, the occurrences of Mₖ attached to Ar are disposed in a meta and/or 1,3-relationship.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuran, benzopyrrole, benzopyridine, etc. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system. In certain embodiments, Cy′ represents a benzo(b)thien-2-yl, preferably a 3-chloro-benzo(b)thien-2-yl, 3-fluoro-benzo(b)thien-2-yl, or 3-methyl-benzo(b)thien-2-yl, e.g., wherein the benzo ring is substituted with from 14 substituents selected from halogen, nitro, cyano, methyl (e.g., including halomethyl, such as CHCl₂ and CF₃), and ethyl (e.g., including haloethyl, such as CH₂CCl₃, C₂F₅, etc.), preferably from halogen and methyl (e.g., including halomethyl, such as CHCl₂ and CF₃). In certain such embodiments Cy′ represents a 3-chloro-benzo(b)thien-2-yl, 3-fluoro-benzo(b)thien-2-yl, or 3-methyl-benzo(b)thien-2-yl wherein the benzo ring is substituted with fluoro at the 4-position (peri to the 3-substituent on the thienyl ring) and, optionally, at the 7-position (‘peri’ to the S of the thienyl ring).

In certain embodiments, Cy represents a substituted or unsubstituted non-aromatic carbocyclic or heterocyclic ring, i.e., including at least one sp hybridized atom, and preferably a plurality of sp hybridized atoms. In certain embodiments, Cy is a 5- to 7-membered ring. In certain embodiments, Cy is directly attached to N and/or to NR₂. In embodiments wherein Cy is a six-membered ring directly attached to N and bears an amino substituent at the 4 position of the ring relative to N, the N and amino substituents may be disposed trans on the ring.

In certain embodiments, X is selected from —C(═O)—, —C(═S)—, and —S(O₂)—.

In certain embodiments, substituents on Ar or Z, where Z is an aryl or heteroaryl ring, are selected from halogen, lower alkyl, lower alkenyl, aryl, heteroaryl, carbonyl, thiocarbonyl, ketone, aldehyde, amino, acylamino, cyano, nitro, hydroxyl, sulfonyl, sulfoxido, sulfate, sulfonate, sulfamoyl, sulfonamido, —(CH₂)palkyl, —(CH₂)palkenyl, —(CH₂)ₚalkynyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein n and p, individually for each occurrence, represent integers from 0 to 10, preferably from 0 to 5.

In certain embodiments, Z is directly attached to Ar, or attached to Ar through a chain of one or two atoms. In certain embodiments, Z-Y-M, taken together, is absent.

Antibodies

In one embodiment of the invention described herein, the agent to stimulate the Hh signaling pathway is an antibody or fragment thereof.

One embodiment of the invention is an antibody raised against an inhibitor of a Hh polypeptide that binds to a Hh polypeptide in competition with a Patched protein, a proposed Hh receptor. Certain antibodies against such an inhibitor mimics the region of a Hh polypeptide which binds to the inhibitor, which may be a region that binds to the Hh receptor. Consequently, such antibodies bind to a Patched protein and elicit similar kind of response in a cell as a Hh polypeptide does.

Another embodiment of the invention is an anti-idiotypic antibody. An anti-idiotypic antibody is raised against a primary antibody. Certain anti-idiotypic antibodies mimic the internal image of the epitope for the primary antibody, thereby also mimicking the activity of the antigen against which the primary antibody has been raised. See, for example, Ma, J. et al., (2002) Japan. J Cancer Res. 93(1):78-84; Depraetere, H. et al., (2000) Eur. J. Biochem. 267(8): 2260-7; Rajeshwari, K. and Karande, A. A., (1999) Immunol. Invest. 28(2-3):103-14. Anti-idiotypic antibodies against an antibody that is specific to the site of a Hh polypeptide involved in functional binding to its receptor, mirror the structure of such a site on a Hh polypeptide. Therefore, such anti-idiotypic antibodies also bind to a receptor for a Hh polypeptide and elicit biologically relevant responses. Anti-idiotypic antibodies are produced in a similar manner to producing any antibody.

Antibodies useful in the present invention may be monoclonal or polyclonal antibodies. As used herein, “monoclonal antibody,” also designated as mAb, is used to describe antibody molecules whose primary sequences are essentially identical and which exhibit the same antigenic specificity. Monoclonal antibodies may be produced by hybridoma, recombinant, transgenic or other techniques known to one skilled in the art. In addition, methods exist to produce monoclonal antibodies in transgenic animals or plants (Pollock et al., J. Immunol. Methods, 231:147, 1999; Russell, Curr. Top. Microbiol. Immunol. 240:119, 1999).

In one embodiment, the portion of the antibody comprises a light chain of the antibody. As used herein, “light chain” means the smaller polypeptide of an antibody molecule composed of one variable domain (VL) and one constant domain (CL), or fragments thereof. In one embodiment, the portion of the antibody comprises a heavy chain of the antibody. As used herein, “heavy chain” means the larger polypeptide of an antibody molecule composed of one variable domain (VH) and three or four constant domains (CH1, CH2, CH3, and CH4), or fragments thereof. In one embodiment, the portion of the antibody comprises a Fab portion of the antibody. As used herein, “Fab” means a monovalent antigen binding fragment of an immunoglobulin that consists of one light chain and part of a heavy chain. In one embodiment, the portion of the antibody comprises a F(ab′)₂ portion of the antibody. As used herein, “F(ab′)2 fragment” means a bivalent antigen binding fragment of an immunoglobulin that consists of both light chains and part of both heavy chains. Fab and F(ab′)2 can be obtained by brief pepsin digestion or recombinant methods. In one embodiment, the portion of the antibody comprises one or more CDR domains of the antibody. As used herein, “CDR” or “complementarity determining region” means a highly variable sequence of amino acids in the variable domain of an antibody, which directly interacts with the epitope of the antigen. Variable domains of an antibody also contains framework regions (FRs), which maintain the tertiary structure of the paratope (see, in general, Clark, 1986; Roitt, 1991). In both the heavy chain Fd fragment and the light chain of IgG immunoglobulins, there are four framework regions (FR₁ through FR₄) separated respectively by three complementarity determining regions (CDR₁ through CDR₃). The CDRs, and in particular the CDR₃ regions, and more particularly the heavy chain CDR₃, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

The antibody may be a human or nonhuman antibody. The nonhuman antibody may be “humanized” by recombinant methods to reduce its immunogenicity in man. Methods for humanizing antibodies are known to those skilled in the art. As used herein, “humanized” describes antibodies wherein some, most or all of the amino acids outside the CDR regions are replaced with corresponding amino acids derived from human immunoglobulin molecules. In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody would retain a similar antigenic specificity as the original antibody.

Methods of humanization include, but are not limited to, those described in U.S. Pat. Nos. 4,816,567, 5,225,539, 5,585,089, 5,693,761, 5,693,762 and 5,859,205, which are hereby incorporated by reference. One of ordinary skill in the art will be familiar with other methods for antibody humanization.

Using certain methods of humanization, the affinity and/or specificity of binding of the antibody may be increased using methods of “directed evolution”, as described by Wu et al., J. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

RNA Interference

In one embodiment the Hh agonists are RNA interference (RNAi) molecules. RNAi constructs comprise double stranded RNA that can specifically block expression of a target gene. Accordingly, RNAi constructs that specifically block expression of a gene that negatively regulates the Hh signaling pathway can act as an agonist of the Hh signaling pathway. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation; however, the biochemical mechanisms are currently an active area of research. Despite some uncertainty regarding the mechanism of action, RNAi provides a useful method of inhibiting gene expression in vitro or in vivo.

In preferred embodiments, hh RNAi agonists of the invention are siRNA, either transcribed from a DNA vector encoding a short hairpin (stem-loop) siRNA, a synthetic siRNA, or longer dsRNA which can be further processed to shorter siRNA (such as 21-23 nucleotides), encoding sequences that interfere with the expression of negative control elements of the Hh signaling pathway, such as Patched or Gli-3.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 base pairs, or 1 in 10 base pairs, or 1 in 20 base pairs, or 1 in 50 base pairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Exemplary Targets of RNAi

The genes listed below are negative regulators of the Hh signaling pathway. A hh RNAi agonist inhibiting a negative regulator will be useful to up-regulate the Hh signaling, for example, in conditions involving hypoactivity of Hh signaling, or when it is desirable to upregulate Hh pathway signaling.

TABLE 2
Negative Regulators of Hedgehog Signaling

| Drosophila | |
|---|---|
| (Acc. No.) | Other Species (Acc. No.) |
| Ptc (M28999) | Human PTC1 (U59464); human PTC2 (AF091501); mouse Ptc1 (U46155); rat |
| | Ptc1 (AF079162); Xenopus Ptc1 (AF302765); chicken Ptc1 (U40074); |
| | zebrafish Ptc1 (X98883); Japanese firebelly newt Ptc1 (AB000848); mouse |
| | Ptc2 (AB010833); chicken Ptc2 (AF409095); Xenopus Ptc2 (AB037688); |
| | zebrafish Ptc2 (AJ007742); Japanese firebelly newt Ptc2 (AB000846) |
| Cos2 | Human homolog (AY237538); rat homolog (XM_218828); mouse homolog |
| (AF019250) | (XM_133575); Anopheles gambiae str. PEST homolog (XM_309818). |
| Su(fu) | Human SUFU (NM_016169); mouse homolog (AJ131692); rat homolog |
| (NM_080502) | (XM_219957); chicken homolog (AF487888); Anopheles gambiae str. PEST |
| | homolog (XM_321114); zebrafish homolog (BC045348). |
| Sgg (X70862) | Human GSK3α (L33801); mouse GSK3α (AF156099); rat GSK3α (X53428); |
| | zebrafish GSK3α (AB032265); Xenopus GSK3α (U31862). |
| Pka-C1 | Human PKA-C1 (X07767, M34181, M34182); rat homolog (X57986); mouse |
| (AY069425) | homolog (BC003238); sheep homolog (AF238979); bovine homolog |
| | (X67154); pig homolog (X05998); rabbit homolog (AF367428;); hamster |
| | homolog (M63311); Xenopus homolog (AJ413219). |
| CK1α | Human homolog (X80693); mouse homolog (BC019740); rat homolog |
| (AΨ069346) | (U77582); chicken homolog (AF042862); sheep homolog (AB050945); bovine |
| | homolog (AB050944); pig homolog (F22872). |
| Slmb | Human homolog (AF101784; AF176022); mouse homolog (AF391190); |
| (AF032878) | Xenopus (M98268); chicken (AF113946). |

* Nucleotide sequence accession numbers from the public databases are listed in “( ).”

Patched inhibits a second membrane-bound protein, Smoothened, in the absence of Hh polypeptide. The association of Patched and Smoothened enables an intracellular high-molecular-weight protein complex, which includes the kinesin-related molecule Costal2 (Cos2), a serine-threonine protein kinase Fused (Fu), and the protein Suppressor of Fused [Su(fu)], to promote the proteolytic processing of full-length Cubitus interruptus (Ci 155), thereby generating a transcriptional repressor Ci75. Although not yet proven to interact directly with this high-molecular weight complex, protein kinase A (PKA), glycogen synthase kinase 3 (GSK3), and casein kinase 1α (CK1α) also modify Ci to regulate its cleavage. This process also depends on Slimb. Binding of Hh to Ptc relieves inhibition of Smo and, by an unknown mechanism, Smo suppresses the Ci-processing activity of the cytoplasmic complex. Unprocessed Ci 155 then translocates to the nucleus, where it activates the expression of specific target genes.

Sporadic tumors in humans demonstrated a loss of both functional alleles of patched. Of twelve tumors in which patched mutations were identified with a single strand conformational polymorphism screening assay, nine had chromosomal deletion of the second allele and the other three had inactivating mutations in both alleles (Gailani, supra). Most of the identified mutations resulted in premature stop codons or frame shifts (Lench, N. J. et al. (1997) Hum. Genet. 100(5-6): 497-502). In addition, there are several identified mutations that are point mutations leading to amino acid substitutions in either extracellular or cytoplasmic domains. The alterations did not occur in the corresponding germ line DNA. An example of Drosophila patched gene is represented in SEQ ID No:23.

The involvement of patched in the inhibition of gene expression and the occurrence of frequent allelic deletions of patched in BCC support a tumor suppressor function for this gene. Its role in the regulation of gene families known to be involved in cell signaling and intercellular communication provides a possible mechanism of tumor suppression.

Recently, Lum et al. (Science (2003) 299: 2039-2045) identified several additional members of the Hh signaling pathway. Using both in vitro and in vivo assays, these authors identified four genes whose products were not previously recognized as having specific roles in Hh signaling: Among the four, CK1α is a negative regulator, while other three are positive regulators. CK1α is a positive regulator of Ci cleavage, a process that generates its repressor form (Price and Kalderon (2002) Cell 108: 823-835, FIG. 1). Thus CK1α is a negative regulator of Hh signaling.

All Hh signaling pathway genes in various species can be routinely obtained from public and proprietary databases, such as GenBank, EMBL, FlyBase, to name but a few. In certain organisms, such as human and Drosophila, the whole genome is sequenced, and sequence comparison programs, such as the BLAST series of programs offered online at the NCBI website can be used to retrieve the most updated sequences of any known Hh signaling pathway genes. The following table list several representative members of the known Hh signaling pathway genes in various species. It is by no means exhaustive, and should not be viewed as limiting in any sense. Rather, it serves as a useful starting point for an exhaustive search, which a skilled artisan would be able to perform these searches using routine biotechniques. Some genes may have several different database entries with different accession numbers, but are nonetheless same or almost the same in sequence. Regardless, only one entry for each gene is provided in the table above.

Exemplary RNAi Constructs

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNA are double stranded, and may include short overhangs at each end. Preferably, the overhangs are 1-6 nucleotides in length at the 3′ end. It is known in the art that the siRNAs can be chemically synthesized, or derived from a longer double-stranded RNA or a hairpin RNA. The siRNAs have significant sequence similarity to a target RNA so that the siRNAs can pair to the target RNA and result in sequence-specific degradation of the target RNA through an RNA interference mechanism. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine is tolerated and does not affect the efficiency of RNAi. The absence of a 2′ hydroxyl significantly enhances the nuclease resistance of the overhang in tissue culture medium and may be beneficial in vivo.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

In certain embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (i.e., hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al. (2002) Genes Dev., 16:948-58; McCaffrey et al. (2002) Nature,418:38-9; McManus et al., (2002) RNA, 8:842-50; Yu et al. (2002) Proc. Natl. Acad. Sci. USA, 99:6047-52). Preferably, such hairpin RNAs are engineered in cells or in an animal to ensure continuous and stable suppression of a desired gene. It is known in the art that siRNAs can be produced by processing a hairpin RNA in the cell.

Preparation of RNAi Constructs

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc. Natl. Acad. Sci. USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides. The double-stranded structure may be formed by a single self-complementary RNA strand or two complementary RNA strands. RNA duplex formation may be initiated either inside or outside the cell.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleofide can be introduced by in vitro enzymatic or organic synthesis.

Methods of chemically modifying RNA molecules can be adapted for modifying RNAi constructs (see, for example, Heidenreich et al. (1997) Nucleic Acids Res., 25:776-780; Wilson et al. (1994) J. Mol. Recog., 7:89-98; Chen et al. (1995) Nucleic Acids Res., 23:2661-2668; Hirschbein et al. (1997) Antisense Nucleic Acid Drug Dev. 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, α-configuration).

Delivery of RNAi Constructs

The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional (or convergent) transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell. Also see Tran et al., (2003) BMC Biotechnology 3: 21 (incorporated herein by reference).

Compositions

One aspect of the present invention provides pharmaceutical compositions comprising an agonist of the Hh signaling pathways. The pharmaceutical compositions comprise a Hh polypeptide or its functional equivalent, or an agonist of Hh activity. The pharmaceutical compositions may also comprise an antagonist of the negative feedback system or of repressive elements of the Hh signaling pathway. The pharmaceutical compositions may further comprise additional therapeutic agents, such as neuronal growth factors or neurotrophic factors.

In still another aspect, the invention relates to a method for preparing a pharmaceutical composition, comprising combining a Hh agonist, optionally an additional pharmaceutically active component, and a pharmaceutically acceptable excipient in a composition for simultaneous administration of the drugs.

This invention provides such agents described herein, and the pharmaceutical compositions may additionally comprise pharmaceutically acceptable carriers. Pharmaceutically acceptable carriers are well known to those skilled in the art. Such pharmaceutically acceptable carriers may include but are not limited to a diluent, an aerosol, a topical carrier, an aqueous solution, a non-aqueous solution or a solid carrier. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

For embodiments wherein the agents are polypeptides and antibodies, the pharmaceutical composition may be formulated for administration with a biologically acceptable medium, such as water, buffered saline, polyol (for example, glycerol, propylene glycol, liquid polyethylene glycol and the like) or suitable mixtures thereof. In preferred embodiments, the polypeptide is dispersed in lipid formulations, such as micelles, which closely resemble the lipid composition of natural cell membranes to which the protein is to be delivered. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the Hh signaling pathway agonist, its use in the pharmaceutical preparation of the invention is contemplated. Suitable vehicles and their formulation inclusive of various proteins are described, for example, in the book Remington's Pharmaceutical Sciences (Remington's Pharmaceutical Sciences. Mack Publishing Company, Easton, Pa., USA 1985).

For such administration, penetrants appropriate to the barrier to be permeated are used in the formulation with the polypeptide. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the proteins of the invention are formulated into ointments, salves, gels, or creams as generally known in the art.

In accordance with the subject method, expression constructs of the subject polypeptides (and endothelialization polypeptide as appropriate) may be administered in any biologically effective carrier, e.g. any formulation or composition capable of effectively transfecting cells in vivo with a recombinant fusion gene. Approaches include insertion of the subject fusion gene in viral vectors including recombinant retroviruses, adenovirus, adeno-associated virus, and herpes simplex virus-1, or recombinant bacterial or eukaryotic plasmids. Viral vectors can be used to transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically.

The optimum concentration of the agent(s) in the chosen medium can be determined empirically, according to procedures well known to medicinal chemists.

Gene Therapy

One aspect of the present invention provides for methods of treatment of various behavioral and emotional disorders using gene therapy. As used herein, “gene therapy” refers to a therapeutic introduction of nucleic acid into a subject cell so that the nucleic acid may be expressed, resulting in alleviation of ailment.

In another preferred embodiment, the invention feature a nucleic acid which encodes a polypeptide that modulates, e.g., mimics or antagonizes, the biological activity of a Hh polypeptide, which nucleic acid comprises all or a portion of the nucleotide sequence of the coding region of a gene identical or homologous to the nucleotide sequence designated by one of SEQ ID No:1, SEQ ID No:2, SEQ ID No:3, SEQ ID No:4, SEQ ID No:5, SEQ ID No:6, SEQ ID No:7, SEQ ID No:8, or SEQ ID No:9. Preferably, the nucleic acid comprises a Hh-encoding portion that hybridizes under stringent conditions to a coding portion of one or more of the nucleic acids designated by SEQ ID No:1-9.

The term equivalent is understood to include nucleotide sequences encoding functionally equivalent Hh polypeptides or functionally equivalent peptides having an activity of a vertebrate Hh polypeptide such as described herein. Equivalent nucleotide sequences will include sequences that differ by one or more nucleotide substitutions, additions or deletions, such as allelic variants; and will, therefore, include sequences that differ from the nucleotide sequence of the vertebrate hh cDNAs shown in SEQ ID Nos: 1-9 due to the degeneracy of the genetic code. Equivalents will also include nucleotide sequences that hybridize under stringent conditions (i.e., equivalent to about 20-27° C. below the melting temperature (Tm) of the DNA duplex formed in about 1M salt) to the nucleotide sequences represented in one or more of SEQ ID Nos:1-9. In one embodiment, equivalents will further include nucleic acid sequences derived from and evolutionarily related to, a nucleotide sequences shown in any of SEQ ID Nos: 1-9.

In yet a further preferred embodiment, the nucleic acid hybridizes under stringent conditions to a nucleic acid probe corresponding to at least 12 consecutive nucleotides of either sense or antisense sequence of one or more of SEQ ID Nos: 1-9; though preferably to at least 20 consecutive nucleotides; and more preferably to at least 40, 50 or 75 consecutive nucleotides of either sense or antisense sequence of one or more of SEQ ID Nos: 1-9.

The term “equivalent” is understood to include nucleotide sequences encoding functionally equivalent Hh polypeptides or functionally equivalent peptides having an activity of a vertebrate Hh polypeptide such as described herein. Equivalent nucleotide sequences will include sequences that differ by one or more nucleotide substitutions, additions or deletions, such as allelic variants; and will, therefore, include sequences that differ from the nucleotide sequence of the vertebrate hh cDNAs shown in SEQ ID Nos: 1-9 due to the degeneracy of the genetic code. Equivalents will also include nucleotide sequences that hybridize under stringent conditions (i.e., equivalent to about 20-27° C. below the melting temperature (Tm) of the DNA duplex formed in about 1M salt) to the nucleotide sequences represented in one or more of SEQ ID Nos: 1-9. In one embodiment, equivalents will further include nucleic acid sequences derived from and evolutionarily related to, a nucleotide sequences shown in any of SEQ ID Nos: 1-9.

Preferred nucleic acids for use in gene therapy of the present invention encode a vertebrate Hh polypeptide comprising an amino acid sequence at least 60% homologous, more preferably 70% homologous and most preferably 80% homologous with an amino acid sequence selected from SEQ ID Nos: 10-18. Nucleic acids which encode polypeptides at least about 90%, more preferably at least about 95%, and most preferably at least about 98-99% homology with an amino acid sequence represented in one of SEQ ID Nos: 10-18 are also within the scope of the invention. In one embodiment, the nucleic acid is a cDNA encoding a peptide having at least one activity of the subject vertebrate Shh polypeptide. Preferably, the nucleic acid includes all or a portion of the nucleotide sequence corresponding to the coding region of SEQ ID Nos: 1-9.

With respect to functionally equivalent fragments of sonic clones, a preferred nucleic acid encodes a polypeptide including a Hh portion having molecular weight of approximately 19 kDa and which polypeptide can modulate, e.g., mimic or antagonize, a Hh biological activity. Preferably, the polypeptide encoded by the nucleic acid comprises an amino acid sequence identical or homologous to an amino acid sequence designated in one of SEQ ID No:10, SEQ ID No:11, SEQ ID No:12, SEQ ID No:13, SEQ ID No:14, SEQ ID No:15, SEQ ID No:16, SEQ ID No:17, or SEQ ID No:18. More preferably, the polypeptide comprises an amino acid sequence designated in SEQ ID No:21.

A preferred nucleic acid encodes a Hh polypeptide comprising an amino acid sequence represented by the formula A-B wherein, A represents all or the portion of the amino acid sequence designated by residues 1-168 of SEQ ID No:21; and B represents at least one amino acid residue of the amino acid sequence designated by residues 169-221 of SEQ ID No:21; wherein A and B together represent a contiguous polypeptide sequence designated by SEQ ID No:21. Preferably, B can represent at least five, ten or twenty amino acid residues of the amino acid sequence designated by residues 169-221 of SEQ ID No:21.

To further illustrate, another preferred nucleic acid encodes a polypeptide comprising an amino acid sequence represented by the formula A-B, wherein A represents all or the portion of the amino acid sequence designated by residues 24-193 of SEQ ID No:15; and B represents at least one amino acid residue of the amino acid sequence designated by residues 194-250 of SEQ ID No:15; wherein A and B together represent a contiguous polypeptide sequence designated in SEQ ID No:15, and the polypeptide modulates, e.g., agonizes or antagonizes, the biological activity of a Hh polypeptide.

Yet another preferred nucleic acid encodes a polypeptide comprising an amino acid sequence represented by the formula A-B, wherein A represents all or the portion, e.g., 25, 50, 75 or 100 residues, of the amino acid sequence designated by residues 25-193, or analogous residues thereof, of a vertebrate Hh polypeptide identical or homologous to SEQ ID No:13; and B represents at least one amino acid residue of the amino acid sequence designated by residues 194-250, or analogous residues thereof, of a vertebrate Hh polypeptide identical or homologous to SEQ ID No:13; wherein A and B together represent a contiguous polypeptide sequence designated in SEQ ID No:13.

Another preferred nucleic acid encodes a polypeptide comprising an amino acid sequence represented by the formula A-B, wherein A represents all or the portion, e.g., 25, 50, 75 or 100 residues, of the amino acid sequence designated by residues 23-193 of SEQ ID No:11; and B represents at least one amino acid residue of the amino acid sequence designated by residues 194-250 of SEQ ID No:11; wherein A and B together represent a contiguous polypeptide sequence designated in SEQ ID No:11, and the polypeptide modulates, e.g., agonizes or antagonizes, the biological activity of a Hh polypeptide.

Another preferred nucleic acid encodes a polypeptide comprising an amino acid sequence represented by the formula A-B, wherein A represents all or the portion, e.g., 25, 50, 75 or 100 residues, of the amino acid sequence designated by residues 28-197 of SEQ ID No:12; and B represents at least one amino acid residue of the amino acid sequence designated by residues 198-250 of SEQ ID No:12; wherein A and B together represent a contiguous polypeptide sequence designated in SEQ ID No:12, and the polypeptide modulates, e.g., agonizes or antagonizes, the biological activity of a Hh polypeptide.

Yet another preferred nucleic acid encodes a polypeptide comprising an amino acid sequence represented by the formula A-B, wherein A represents all or the portion, e.g., 25, 50 or 75 residues, of the amino acid sequence designated by residues 1-98, or analogous residues thereof, of a vertebrate Hh polypeptide identical or homologous to SEQ ID No:18; and B represents at least one amino acid residue of the amino acid sequence designated by residues 99-150, or analogous residues thereof, of a vertebrate Hh polypeptide identical or homologous to SEQ ID No:18; wherein A and B together represent a contiguous polypeptide sequence designated in SEQ ID No:18.

Another aspect of the invention provides a nucleic acid which hybridizes under high or low stringency conditions to a nucleic acid represented by one of SEQ ID Nos: 1-9. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C.

Nucleic acids, having a sequence that differs from the nucleotide sequences shown in one of SEQ ID No:1, SEQ ID No:2, SEQ ID No:3, SEQ ID No:4, SEQ ID No:5, SEQ ID No:6, SEQ ID No:7, SEQ ID No:8, or SEQ ID No:9, due to degeneracy in the genetic code are also within the scope of the invention. Such nucleic acids encode functionally equivalent peptides (i.e., a peptide having a biological activity of a vertebrate hh polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of a vertebrate hh polypeptide. However, it is expected that DNA sequence polymorphisms that do lead to changes in the amino acid sequences of the subject hh polypeptides will exist among vertebrates. One skilled in the art will appreciate that these variations in one or more nucleotides (up to about 3-5% of the nucleotides) of the nucleic acids encoding polypeptides having an activity of a vertebrate hh polypeptide may exist among individuals of a given species due to natural allelic variation. As used herein, a hh gene fragment refers to a nucleic acid having fewer nucleotides than the nucleotide sequence encoding the entire mature form of a vertebrate Hh polypeptide yet which (preferably) encodes a polypeptide which retains some biological activity of the full length protein.

In an illustrative embodiment, alignment of exons 1, 2 and a portion of exon 3 encoded sequences (e.g. the N-terminal approximately 221 residues of the mature protein) of each of the Shh clones produces a degenerate set of Shh polypeptides represented by the general formula:

C-G-P-G-R-G-X(1)-G-X(2)-R-R-H-P-K-K-L-T-P-L-A-Y-K-Q-F-I-P-N-V-A-E-K-T-L-G-A-S-G-R-Y-E-G-K-I-X(3)-R-N-S-E-R-F-K-E-L-T-P-N-Y-N-P-D-I-I-F-K-D-E-E-N-T-G-A-D-R-L-M-T-Q-R-C-K-D-K-L-N-X(4)-L-A-I-S-V-M-N-X(5)-W-P-G-V-X(6)-L-R-V-T-E-G-W-D-E-D-G-H-H-X(7)-E-E-S-L-H-Y-E-G-R-A-V-D-I-T-T-S-D-R-D-X(8)-S-K-Y-G-X(9)-L-X(10)-R-L-A-V-E-A-G-F-D-W-V-Y-Y-E-S-K-A-H-I-H-C-S-V-K-A-E-N-S-V-A-A-K-S-G-G-C-F-P-G-S-A-X(11)-V-X(12)-L-X(13)-X(14)-G-G-X(15)-K-X-(16)-V-K-D-L-X(17)-P-G-D-X(18)-V-L-A-A-D-X(19)-X(20)-G-X(21)-L-X(22)-X(23)-S-D-F-X(24)-X(25)-F-X(26)-D-R (SEQ ID No: 21),
wherein each of the degenerate positions “X” can be an amino acid which occurs in that position in one of the human, mouse, chicken or zebrafish Shh clones, or, to expand the library, each X can also be selected from amongst amino acid residue which would be conservative substitutions for the amino acids which appear naturally in each of those positions. For instance, Xaa(1) represents Gly, Ala, Val, Leu, Ile, Phe, Tyr or Trp; Xaa(2) represents Arg, His or Lys; Xaa(3) represents Gly, Ala, Val, Leu, Ile, Ser or Thr; Xaa(4) represents Gly, Ala, Val, Leu, Ile, Ser or Thr; Xaa(5) represents Lys, Arg, His, Asn or Gln; Xaa(6) represents Lys, Arg or His; Xaa(7) represents Ser, Thr, Tyr, Trp or Phe; Xaa(8) represents Lys, Arg or His; Xaa(9) represents Met, Cys, Ser or Thr; Xaa(10) represents Gly, Ala, Val, Leu, Ile, Ser or Thr; Xaa(11) represents Leu, Val, Met, Thr or Ser; Xaa(12) represents His, Phe, Tyr, Ser, Thr, Met or Cys; Xaa(13) represents Gln, Asn, Glu, or Asp; Xaa(14) represents His, Phe, Tyr, Thr, Gln, Asn, Glu or Asp; Xaa(15) represents Gln, Asn, Glu, Asp, Thr, Ser, Met or Cys; Xaa(16) represents Ala, Gly, Cys, Leu, Val or Met; Xaa(17) represents Arg, Lys, Met, Ile, Asn, Asp, Glu, Gln, Ser, Thr or Cys; Xaa(18) represents Arg, Lys, Met or Ile; Xaa(19) represents Ala, Gly, Cys, Asp, Glu, Gln, Asn, Ser, Thr or Met; Xaa(20) represents Ala, Gly, Cys, Asp, Asn, Glu or Gln; Xaa(21) represents Arg, Lys, Met, Ile, Asn, Asp, Glu or Gln; Xaa(22) represent Leu, Val, Met or Ile; Xaa(23) represents Phe, Tyr, Thr, His or Trp; Xaa(24) represents Ile, Val, Leu or Met; Xaa(25) represents Met, Cys, Ile, Leu, Val, Thr or Ser; Xaa(26) represents Leu, Val, Met, Thr or Ser. In an even more expansive library, each X can be selected from any amino acid.

In similar fashion, alignment of each of the human, mouse, chicken and zebrafish hh clones (FIG. 5B), can provide a degenerate polypeptide sequence represented by the general formula:

C-G-P-G-R-G-X(1)-X(2)-X(3)-R-R-X(4)-X(5)-X(6)-P-K-X(7)-L-X(8)-P-L-X(9)-Y-K-Q-F-X(10)-P-X(11)-X(12)-X(13)-E-X(14)-T-L-G-A-S-G-X(15)-X(16)-E-G-X(17)-X(18)-X(19)-R-X(20)-S-E-R-F-X(21)-X(22)-L-T-P-N-Y-N-P-D-I-I-F-K-D-E-E-N-X(23)-G-A-D-R-L-M-T-X(24)-R-C-K-X(25)-X(26)-X(27)-N-X(28)-L-A-I-S-V-M-N-X(29)-W-P-G-V-X(30)-L-R-V-T-E-G-X(31)-D-E-D-G-H-H-X(32)-X(33)-X(34)-S-L-H-Y-E-G-R-A-X(35)-D-I-T-T-S-D-R-D-X(36)-X(37)-K-Y-G-X(38)-L-X(39)-R-L-A-V-E-A-G-F-D-W-V-Y-Y-E-S-X(40)-X(41)-H-X(42)-H-X(43)-S-V-K-X(44)-X(45) (SEQ ID No: 22),
wherein, as above, each of the degenerate positions “X” can be an amino acid which occurs in a corresponding position in one of the wild-type clones, and may also include amino acid residue which would be conservative substitutions, or each X can be any amino acid residue. In an exemplary embodiment, Xaa(1) represents Gly, Ala, Val, Leu, Ile, Pro, Phe or Tyr; Xaa(2) represents Gly, Ala, Val, Leu or Ile; Xaa(3) represents Gly, Ala, Val, Leu, Ile, Lys, His or Arg; Xaa(4) represents Lys, Arg or His; Xaa(5) represents Phe, Trp, Tyr or an amino acid gap; Xaa(6) represents Gly, Ala, Val, Leu, Ile or an amino acid gap; Xaa(7) represents Asn, Gln, His, Arg or Lys; Xaa(8) represents Gly, Ala, Val, Leu, Ile, Ser or Thr; Xaa(9) represents Gly, Ala, Val, Leu, Ile, Ser or Thr; Xaa(10) represents Gly, Ala, Val, Leu, Ile, Ser or Thr; Xaa(11) represents Ser, Thr, Gln or Asn; Xaa(12) represents Met, Cys, Gly, Ala, Val, Leu, Ile, Ser or Thr; Xaa(13) represents Gly, Ala, Val, Leu, Ile or Pro; Xaa(14) represents Arg, His or Lys; Xaa(15) represents Gly, Ala, Val, Leu, Ile, Pro, Arg, His or Lys; Xaa(16) represents Gly, Ala, Val, Leu, Ile, Phe or Tyr; Xaa(17) represents Arg, His or Lys; Xaa(18) represents Gly, Ala, Val, Leu, Ile, Ser or Thr; Xaa(19) represents Thr or Ser; Xaa(20) represents Gly, Ala, Val, Leu, Ile, Asn or Gln; Xaa(21) represents Arg, His or Lys; Xaa(22) represents Asp or Glu; Xaa(23) represents Ser or Thr; Xaa(24) represents Glu, Asp, Gln or Asn; Xaa(25) represents Glu or Asp; Xaa(26) represents Arg, His or Lys; Xaa(27) represents Gly, Ala, Val, Leu or Ile; Xaa(28) represents Gly, Ala, Val, Leu, Ile, Thr or Ser; Xaa(29) represents Met, Cys, Gln, Asn, Arg, Lys or His; Xaa(30) represents Arg, His or Lys; Xaa(31) represents Trp, Phe, Tyr, Arg, His or Lys; Xaa(32) represents Gly, Ala, Val, Leu, Ile, Ser, Thr, Tyr or Phe; Xaa(33) represents Gln, Asn, Asp or Glu; Xaa(34) represents Asp or Glu; Xaa(35) represents Gly, Ala, Val, Leu, or Ile; Xaa(36) represents Arg, His or Lys; Xaa(37) represents Asn, Gln, Thr or Ser; Xaa(38) represents Gly, Ala, Val, Leu, Ile, Ser, Thr, Met or Cys; Xaa(39) represents Gly, Ala, Val, Leu, Ile, Thr or Ser; Xaa(40) represents Arg, His or Lys; Xaa(41) represents Asn, Gln, Gly, Ala, Val, Leu or Ile; Xaa(42) represents Gly, Ala, Val, Leu or Ile; Xaa(43) represents Gly, Ala, Val, Leu, Ile, Ser, Thr or Cys; Xaa(44) represents Gly, Ala, Val, Leu, Ile, Thr or Ser; and Xaa(45) represents Asp or Glu.

The functional equivalent polypeptides can be selected from these sequences using protocols well known in the art to screen a combinatorial expression library.

A preferred approach for in vivo introduction of nucleic acid encoding one of the subject polypeptides into a cell is by use of a viral vector containing nucleic acid, e.g. a cDNA, encoding the gene product. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a CKI polypeptide, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

In choosing retroviral vectors as a gene delivery system for the subject proteins, it is important to note that a prerequisite for the successful infection of target cells by most retroviruses, and therefore of stable introduction of the recombinant gene, is that the target cells must be dividing. Such limitation on infection can be beneficial when the tissue surrounding the target cells does not undergo extensive cell division and is therefore refractory to infection with retroviral vectors.

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al. (1989) Proc. Nat. Acad. Sci. USA 86:9079-9083; Julan et al. (1992) J. Gen. Virol. 73:3251-3255; and Goud et al. (1983) Virology 163:251-254); or coupling cell surface ligands to the viral env proteins (Neda et al. (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Moreover, use of retroviral gene delivery can be further enhanced by the use of tissue- or cell-specific transcriptional regulatory sequences which control expression of the fusion gene of the retroviral vector.

Another viral vector system useful for delivery of the subject polypeptides is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics Microbiol. Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see, for example, Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al. (1988) BioTechniques 6:616; Rosenfeld et al. (1991) Science 252:431-434; and Rosenfeld et al. (1992) Cell 68:143-155). Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses can be advantageous in certain circumstances in that they are not capable of infecting non-dividing cells and can be used to infect a wide variety of cell types, including endothelial cells (Lemarchand et al. (1992) Proc. Natl. Acad. Sci. USA 89:6482-6486), and smooth muscle cells (Quantin et al. (1992) Proc. Natl. Acad. Sci. USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the inserted fusion gene can be under control of, for example, the E1A promoter, the major late promoter (MLP) and associated leader sequences, the E3 promoter, or exogenously added promoter sequences.

Other viral vector systems that may have application in gene therapy have been derived from herpes virus, Vaccinia virus, and several RNA viruses. In particular, herpes virus vectors may provide a unique strategy for persistent expression of the subject fusion proteins in cells of the central nervous system and ocular tissue (Pepose et al. (1994) Invest. Ophthalmol. Vis. Sci. 35:2662-2666)

In addition to viral transfer methods, such as those illustrated above, non-viral methods can also be employed to cause expression of the subject proteins in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the gene by the targeted cell. Exemplary gene delivery systems of this type include liposomal derived systems, poly-lysine conjugates, and artificial viral envelopes.

In a representative embodiment, a gene encoding one of the subject proteins can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075). For example, lipofection of neuroglioma cells can be carried out using liposomes tagged with monoclonal antibodies against glioma-associated antigen (Mizuno et al. (1992) Neurol. Med. Chir. 32:873-876).

In yet another illustrative embodiment, the gene delivery system comprises an antibody or cell surface ligand which is cross-linked with a gene binding agent such as poly-lysine (see, for example, PCT publications WO93/04701, WO92/22635, WO92/20316, WO92/19749, and WO92/06180). For example, the subject gene construct can be used to transfect hepatocytic cells in vivo using a soluble polynucleotide carrier comprising an asialoglycoprotein conjugated to a polycation, e.g. poly-lysine (see U.S. Pat. No. 5,166,320). It will also be appreciated that effective delivery of the subject nucleic acid constructs via receptor-mediated endocytosis can be improved using agents which enhance escape of the gene from the endosomal structures. For instance, whole adenovirus or fusogenic peptides of the influenza HA gene product can be used as part of the delivery system to induce efficient disruption of DNA-containing endosomes (Mulligan et al. (1993) Science 260-926; Wagner et al. (1992) Proc. Nat. Acad. Sci. USA 89:7934; and Christiano et al. (1993) Proc. Nat. Acad. Sci. USA 90:2122).

In clinical settings, the gene delivery systems can be introduced into a patient by any of a number of methods, each of which is familiar in the art. For instance, a pharmaceutical preparation of the gene delivery system can be introduced systemically, e.g. by intravenous injection, and specific transduction of the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) Proc. Nat. Acad. Sci. USA 91: 3054-3057).

Moreover, the pharmaceutical preparation can consist essentially of the gene delivery system in an acceptable diluent, or can comprise a slow release matrix in which the gene delivery vehicle is imbedded. Alternatively, where the complete gene delivery system can be produced in tact from recombinant cells, e.g., retroviral packages, the pharmaceutical preparation can comprise one or more cells which produce the gene delivery system. In the case of the latter, methods of introducing the viral packaging cells may be provided by, for example, rechargeable or biodegradable devices. Various slow release polymeric devices have been developed and tested in vivo in recent years for the controlled delivery of drugs, including proteinaceous biopharmaceuticals, and can be adapted for release of viral particles through the manipulation of the polymer composition and form. A variety of biocompatible polymers (including hydrogels), including both biodegradable and non-degradable polymers, can be used to form an implant for the sustained release of the viral particles by cells implanted at a particular target site. Such embodiments of the present invention can be used for the delivery of an exogenously purified virus, which has been incorporated in the polymeric device, or for the delivery of viral particles produced by a cell encapsulated in the polymeric device.

Method of Carrying Out Pharmaceutical Business

In another aspect, the invention relates to a method for conducting a pharmaceutical business, by manufacturing a preparation of a Hh agonist and optionally an additional pharmaceutically active component or a kit including separate formulations of each, and marketing to healthcare providers the benefits of using the preparation or kit in the treatment of behavioral and/or emotional/cognitive disorders.

In yet another aspect, the invention provides a method for conducting a pharmaceutical business, by providing a distribution network for selling the combinatorial preparations and kits, and providing instruction material to patients or physicians for using such preparation or kit in the treatment of behavioral and/or emotional/cognitive disorders.

In still a further aspect, the invention relates to a method for conducting a pharmaceutical business, by determining an appropriate formulation and dosage of a Hh agonist, optionally an additional pharmaceutically active component to be co-administered in the treatment of behavioral and/or emotional/cognitive disorders, conducting therapeutic profiling of identified formulations for efficacy and toxicity in animals, and providing a distribution network for selling a preparation as having an acceptable therapeutic profile. In certain embodiments, the method further includes an additional step of providing a sales group for marketing the preparation to healthcare providers.

In yet another aspect, the invention provides a method for conducting a pharmaceutical business by determining an appropriate formulation and dosage of a Hh agonist, optionally an additional pharmaceutically active component to be co-administered in the treatment of behavioral and/or emotional/cognitive disorders, and licensing, to a third party, the rights for further development and sale of the formulation.

In certain embodiments, the disorders to be treated are movement disorders, including ataxia, corticobasal ganglionic degeneration (CBGD), dyskinesia, dystonia, tremors, hereditary spastic paraplegia, Huntington's disease, multiple sclerosis, multiple system atrophy, myoclonus, Parkinson's disease, progressive supranuclear palsy, restless legs syndrome, Rett syndrome, spasticity, Sydenham's chorea, other choreas, athetosis, ballism, stereotypy, tardive dyskinesia/dystonia, tics, Tourette's syndrome, olivopontocerebellar atrophy (OPCA), diffuse Lewy body disease, hemibalismus, hemi-facial spasm, restless leg syndrome, Wilson's disease, stiff man syndrome, akinetic mutism, psychomotor retardation, painful legs moving toes syndrome, a gait disorder, a drug-induced movement disorder, or other movement disorder.

In other embodiments, the emotional or cognitive disorders are attention deficit disorder (ADD), attention deficit hyperactivity disorder (ADHD), cognitive disorders such as dementias (including age-related or senile dementia, HIV-associated dementia, AIDS dementia complex (ADC), dementia due to HIV encephalopathy, Parkinson's disease, Alzheimer's disease, head trauma, Huntington's disease, Pick's disease, Creutzfeldt-Jakob disease, Anterior Communicating Artery Syndrome, hypoxia, post cardiac surgery, Downs syndrome and stroke) and memory impairment such as due to toxicant exposure or brain injury, age-associated memory impairment, mild cognitive impairment, epilepsy, or mental retardation in children.

In certain embodiments, the disorders are autistic disorders.

In still other embodiments, the behavioral disorders are dyssomnias, parasomnias, sleep disorders associated with medical or psychiatric conditions, or other sleep disorders. In certain preferred embodiments, the dyssomnias are selected from intrinsic sleep disorders, extrinsic sleep disorders, and circadian rhythm sleep disorders. Examples of intrinsic sleep disorders include psychophysiological insomnia, sleep state misperception, idiopathic insomnia, narcolepsy, recurrent hypersomnia, idiopathic hypersomnia, posttraumatic hypersomnia, obstructive sleep apnea syndrome, central sleep apnea syndrome, central alveolar hypoventilation, periodic limb movement disorder, restless leg syndrome (RLS), etc. Examples of extrinsic sleep disorders include inadequate sleep hygiene, environmental sleep disorder, altitude insomnia, adjustment sleep disorder, insufficient sleep syndrome, limit-setting sleep disorder, sleep-onset association disorder, food allergy insomnia, nocturnal eating/drinking syndrome, hypnotic-dependent sleep disorder, stimulant-dependent sleep disorder, alcohol-dependent sleep disorder, toxin-induced sleep disorder, etc. Examples of circadian rhythm sleep disorders include time-zone change (jet lag) syndrome, shift-work sleep disorder, irregular sleep/wake pattern, delayed sleep-phase syndrome, advanced sleep-phase syndrome, non-24-hour sleep/wake disorder, etc.

In certain embodiments, the disorder to be treated is ADHD, and the additional pharmaceutically active component is a dopamine re-uptake inhibitor. In other embodiments, the additional pharmaceutically active component is selected from

In certain embodiments, the treatment is prophylactic treatment to be administered to patients who have been diagnosed as having or at risk of developing the exemplary disorders of above.

Any of the embodiments of the methods for conducting a pharmaceutical business may be adapted, in place of the treatment for memory and cognition disorders, for enhancement of memory and cognition in a subject exhibiting normal range of memory and cognitive function.

All publications and patents cited herein are hereby incorporated by reference in their entirety.

Equivalents

Those skilled in the art will recognize, or be able to ascertain using no more than routine experimentation, many equivalents to the specific embodiments of the invention described herein. Such equivalents are intended to be encompassed by the following claims.

1. A method of treating depression, comprising administering to a patient exhibiting symptoms of depression an agonist of Hh signaling, thereby ameliorating some or all of the symptoms.

2. The method of claim 1, wherein said method ameliorates memory impairment or cognitive impairment associated with depression.

3. A method for prophylactic treatment of a patient at risk of developing depression, comprising administering to said patient an agonist of Hh signaling, thereby preventing depression.

4. A method of enhancing cognitive function in a mammal, comprising administering to the mammal an agonist of a Hh signaling pathway, thereby enhancing cognitive function of the mammal.

5. The method of claim 4, wherein said mammal exhibits cognitive function in a statistically normal range prior to the administration of said agonist.

6. The method of claim 4, wherein said mammal has a deficiency in cognitive function as a result of causes other than Alzheimer's disease prior to the administration of said agonist.

7. The method of claim 4, wherein said mammal does not exhibit hallmarks of Alzheimer's disease in addition to a deficiency in cognitive function.

8. A method of prophylactic treatment of a patient at risk of developing a deficiency in cognition, comprising administering to said mammal an agonist of a Hh signaling pathway, thereby preventing deficiency in cognitive function.

9. A method of enhancing memory in a mammal, comprising administering to the mammal an agonist of a Hh signaling pathway, thereby enhancing memory in the mammal.

10. The method of claim 9, wherein said mammal exhibits memory function in a statistically normal range prior to the administration of said agonist.

11. The method of claim 9, wherein said mammal has a deficiency in memory function as a result of causes other than Alzheimer's disease prior to the administration of said agonist.

12. The method of claim 9, wherein said mammal does not exhibit hallmarks of Alzheimer's disease in addition to a deficiency in memory.

13. A method of prophylactic treatment of a patient at risk of developing a deficiency in memory, comprising administering to said mammal an agonist of a Hh signaling pathway, thereby preventing memory deficiency.

14. A method of treating an emotional disorder characterized by abnormal activity of a central nervous system of a mammal, comprising administering to the mammal an agonist of a Hh signaling pathway, thereby treating the emotional disorder.

15. The method of claim 14, wherein said disorder is depression, anxiety disorder, panic disorder, obsessive compulsive disorders, social anxiety/phobic disorder, or posttraumatic stress syndrome.

16. A method for prophylactic treatment of an emotional disorder characterized by abnormal activity of a central nervous system of a mammal, comprising administering to the mammal an agonist of a Hh signaling pathway, thereby preventing the emotional disorder.

17. The method of claim 16, wherein said disorder is depression anxiety disorder, panic disorder, obsessive compulsive disorders, social anxiety/phobic disorder, or posttraumatic stress syndrome.

18. A method of treating non-Alzheimer's dementia or ADHD, comprising administering to the mammal an agonist of a Hh signaling pathway, thereby treating the behavioral disorder.

19. A method for prophylactic treatment of non-Alzheimer's dementia or ADHD, comprising administering to the mammal an agonist of a Hh signaling pathway, thereby preventing the behavioral disorder.

20. The method of claim 1, wherein the agonist functionally binds to a receptor complex which comprises a Patched receptor for a Hh polypeptide.

21. The method of claim 20, wherein the agonist functionally binds to a Smoothened protein.

22. The method of claim 1, wherein the agonist inhibits a negative feedback or repressive factor within a Hh signaling pathway.

23. The method of claim 22, wherein the agonist interferes with the repressor function of a Patched protein, thereby activating a Hh signaling pathway.

24. The method of claim 23, wherein the agonist disrupts functional interactions between a Patched protein and a Smoothened protein.

25. The method of claim 20, wherein said agent is a small organic molecule.

26. The method of claim 20, wherein said agonist is a functional equivalent of a polypeptide comprising full or partial sequence of any of SEQ ID NOs: 10 to 18.

27. The method of claim 20, wherein said agonist is a polypeptide comprising an amino acid sequence at least 80% identical to any of SEQ ID NOs: 10 to 18, or a fragment thereof, wherein said amino acid sequence or fragment thereof binds to a patched receptor.

28. The method of claim 27, wherein said agonist is a 19 kDa N-terminal fragment of any of SEQ ID NOs: 10 to 18.

29. The method of claim 27, wherein said agonist is a polypeptide at least 70% identical to any of SEQ ID NOs: 10 to 18.

30. The method of claim 20, wherein said polypeptide comprises SEQ ID NO: 15 or a fragment thereof that binds to patched.

31. The method of claim 20, wherein the agonist is an anti-idiotypic antibody which activates Hh signaling pathway.

32. The method of claim 1, wherein the agonist is a vector comprising a nucleic acid that, when expressed, activates Hh signaling.

33. The method of claim 32, wherein said vector comprises a nucleotide sequence encoding a polypeptide which comprises an amino acid sequence at least 70% identical to any of SEQ ID NOs: 10-18.

34. The method of claim 33, wherein said polypeptide comprises an amino acid sequence of SEQ ID NO: 15.

35. The method of claim 32, wherein said vector contains a polynucleotide that hybridizes under conditions comprising 6.0× sodium chloride/sodium citrate (SSC), washing at about 45° C., followed by a wash of 2.0×SSC at 50° C. to a second polynucleotide comprising any of SEQ ID NOs: 1 to 9.

36. The method of claim 22, wherein said agonist is an RNAi construct.

37. The method of claim 36, wherein said vector comprises an RNAi construct and a promoter operatively linked to said RNAi construct, wherein said RNAi construct inhibits a negative feedback or repressive factor within a Hh signaling pathway.

38. The method of claim 36, wherein said RNAi construct is an siRNA.

39. The method of claim 36, wherein said RNAi construct inhibits a gene listed in Table 2.

40. The method of claim 36, wherein said RNAi construct inhibits Gli-3.

41. The method of claim 39, wherein said RNAi construct inhibits patched.

42. The method of claim 22, wherein said agonist is a small organic molecule.

43. The method of claim 25, wherein a pharmaceutical composition comprising said agonist is administered orally.

44. The method of claim 1, wherein a pharmaceutical composition comprising said agonist is administered parenterally.

45. The method of claim 1, wherein a pharmaceutical composition comprising said agonist is administered by injection into a target site.

46. The method of claim 1, further comprising administering an additional pharmaceutically active component.

47. The method of claim 46, wherein said additional pharmaceutically active component is a dopamine reuptake inhibitor.

48-64. (canceled)

65. The method of claim 1, wherein the Hh agonist is a compound having a structure of any of Formulae I-XII.


SEQUENCE LISTING (23 sequences)

SEQ ID NO: 1 - DNA, 1277 bp, Gallus gallus
  CDS (1)...(1275)
atggtcgaaatgctgctgttgacaagaattctcttggtgggcttcatctgcgctcttttagtctcctctgggctgacttgtggaccaggcaggggcattggaaaaaggaggcaccccaaaaagctgaccccgttagcctataagcagtttattcccaatgtggcagagaagaccctaggggccagtggaagatatgaagggaagatcacaagaaactccgagagatttaaagaactaaccccaaattacaaccctgacattatttttaaggatgaagagaacacgggagctgacagactgatgactcagcgctgcaaggacaagctgaatgccctggcgatctcggtgatgaaccagtggcccggggtgaagctgcgggtgaccgagggctgggacgaggatggccatcactccgaggaatcgctgcactacgagggtcgcgccgtggacatcaccacgtcggatcgggaccgcagcaagtacggaatgctggcccgcctcgccgtcgaggccggcttcgactgggtctactacgagtccaaggcgcacatccactgctccgtcaaagcagaaaactcagtggcagcgaaatcaggaggctgcttccctggctcagccacagtgcacctggagcatggaggcaccaagctggtgaaggacctgagccctggggaccgcgtgctggctgctgacgcggacggccggctgctctacagtgacttcctcaccttcctcgaccggatggacagctcccgaaagctcttctacgtcatcgagacgcggcagccccgggcccggctgctactgacggcggcccacctgctctttgtggccccccagcacaaccagtcggaggccacagggtccaccagtggccaggcgctcttcgccagcaacgtgaagcctggccaacgtgtctatgtgctgggcgagggcgggcagcagctgctgccggcgtctgtccacagcgtctcattgcgggaggaggcgtccggagcctacgccccactcaccgcccagggcaccatcctcatcaaccgggtgttggcctcctgctacgccgtcatcgaggagcacagttgggcccattgggccttcgcaccattccgcttggctcaggggctgctggccgccctctgcccagatggggccatccctactgccgccaccaccaccactggcatccattggtactcacggctcctctaccgcatcggcagctgggtgctggatggtgacgcgctgcatccgctgggcatggtggcaccggccagctg

SEQ ID NO: 2 - DNA, 1190 bp, Mus musculus
  CDS (1)...(1190)
atggctctgccggccagtctgttgcccctgtgctgcttggcactcttggcactatctgcccagagctgcgggccgggccgaggaccggttggccggcggcgttatgtgcgcaagcaacttgtgcctctgctatacaagcagtttgtgcccagtatgcccgagcggaccctgggcgcgagtgggccagcggaggggagggtaacaagggggtcggagcgcttccgggacctcgtacccaactacaaccccgacataatcttcaaggatgaggagaacagcggcgcagaccgcctgatgacagagcgttgcaaagagcgggtgaacgctctagccatcgcggtgatgaacatgtggcccggagtacgcctacgtgtgactgaaggctgggacgaggacggccaccacgcacaggattcactccactacgaaggccgtgccttggacatcaccacgtctgaccgtgaccgtaataagtatggtttgttggcgcgcctagctgtggaagccggattcgactgggtctactacgagtcccgcaaccacatccacgtatcggtcaaagctgataactcactggcggtccgagccggaggctgctttccgggaaatgccacggtgcgcttgcggagcggcgaacggaaggggctgagggaactacatcgtggtgactgggtactggccgctgatgcagcgggccgagtggtacccacgccagtgctgctcttcctggaccgggatctgcagcgccgcgcctcgttcgtggctgtggagaccgagcggcctccgcgcaaactgttgctcacaccctggcatctggtgttcgctgctcgcgggccagcgcctgctccaggtgactttgcaccggtgttcgcgcgccgcttacgtgctggcgactcggtgctggctcccggcggggacgcgctccagccggcgcgcgtagcccgcgtggcgcgcgaggaagccgtgggcgtgttcgcaccgctcactgcgcacgggacgctgctggtcaacgacgtcctcgcctcctgctacgcggttctagagagtcaccagtgggcccaccgcgccttcgcccctttgcggctgctgcacgcgctcggggctctgctccctgggggtgcagtccagccgactggcatgcattggtactctcgcctcctttaccgcttggccgaggagttaatgggctg

SEQ ID NO: 3 - DNA, 1281 bp, Mus musculus
  CDS (1)...(1233)
atgtctcccgcctggctccggccccgactgcggttctgtctgttcctgctgctgctgcttctggtgccggcggcgcggggctgcgggccgggccgggtggtgggcagccgccggaggccgcctcgcaagctcgtgcctcttgcctacaagcagttcagccccaacgtgccggagaagaccctgggcgccagcgggcgctacgaaggcaagatcgcgcgcagctctgagcgcttcaaagagctcacccccaactacaatcccgacatcatcttcaaggacgaggagaacacgggtgccgaccgcctcatgacccagcgctgcaaggaccgtctgaactcactggccatctctgtcatgaaccagtggcctggtgtgaaactgcgggtgaccgaaggccgggatgaagatggccatcactcagaggagtctttacactatgagggccgcgcggtggatatcaccacctcagaccgtgaccgaaataagtatggactgctggcgcgcttagcagtggaggccggcttcgactgggtgtattacgagtccaaggcccacgtgcattgctctgtcaagtctgagcattcggccgctgccaagacaggtggctgctttcctgccggagcccaggtgcgcctagagaacggggagcgtgtggccctgtcagctgtaaagccaggagaccgggtgctggccatgggggaggatgggacccccaccttcagtgatgtgcttattttcctggaccgcgagccaaaccggctgagagctttccaggtcatcgagactcaggatcctccgcgtcggctggcgctcacgcctgcccacctgctcttcattgcggacaatcatacagaaccagcagcccacttccgggccacatttgccagccatgtgcaaccaggccaatatgtgctggtatcaggggtaccaggcctccagcctgctcgggtggcagctgtctccacccacgtggcccttgggtcctatgctcctctcacaaggcatgggacacttgtggtggaggatgtggtggcctcctgctttgcagctgtggctgaccaccatctggctcagttggccttctggcccctgcgactgtttcccagtttggcatggggcagctggaccccaagtgagggtgttcactcctaccctcagatgctctaccgcctggggcgtctcttgctagaagagagcaccttccatccactgggcatgtctggggcaggaagctgaagggactctaaccactgccctcctggaactgctgtgcgtggatcc

SEQ ID NO: 4 - DNA, 1313 bp, Mus musculus
  CDS (1)...(1313)
atgctgctgctgctggccagatgttttctggtgatccttgcttcctcgctgctggtgtgccccgggctggcctgtgggcccggcagggggtttggaaagaggcggcaccccaaaaagctgacccctttagcctacaagcagtttattcccaacgtagccgagaagaccctaggggccagcggcagatatgaagggaagatcacaagaaactccgaacgatttaaggaactcacccccaattacaaccccgacatcatatttaaggatgaggaaaacacgggagcagaccggctgatgactcagaggtgcaaagacaagttaaatgccttggccatctctgtgatgaaccagtggcctggagtgaggctgcgagtgaccgagggctgggatgaggacggccatcattcagaggagtctctacactatgagggtcgagcagtggacatcaccacgtccgaccgggaccgcagcaagtacggcatgctggctcgcctggctgtggaagcaggtttcgactgggtctactatgaatccaaagctcacatccactgttctgtgaaagcagagaactccgtggcggccaaatccggcggctgtttcccgggatccgccaccgtgcacctggagcagggcggcaccaagctggtgaaggacttacgtcccggagaccgcgtgctggcggctgacgaccagggccggctgctgtacagcgacttcctcaccttcctggaccgcgacgaaggcgccaagaaggtcttctacgtgatcgagacgctggagccgcgcgagcgcctgctgctcaccgccgcgcacctgctcttcgtggcgccgcacaacgactcggggcccacgcccgggccaagcgcgctctttgccagccgcgtgcgccccgggcagcgcgtgtacgtggtggctgaacgcggcggggaccgccggctgctgcccgccgcggtgcacagcgtgacgctgcgagaggaggaggcgggcgcgtacgcgccgctcacggcgcacggcaccattctcatcaaccgggtgctcgcctcgtgctacgctgtcatcgaggagcacagctgggcacaccgggccttcgcgcctttccgcctggcgcacgcgctgctggccgcgctggcacccgcccgcacggacggcgggggcgggggcagcatccctgcagcgcaatctgcaacggaagcgaggggcgcggagccgactgcgggcatccactggtactcgcagctgctctaccacattggcacctggctgttggacagcgagaccatgcatcccttgggaatggcggtcaagtccagctg

SEQ ID NO: 5 - DNA, 1256 bp, Brachydanio rerio
  CDS (1)...(1256)
atgcggcttttgacgagagtgctgctggtgtctcttctcactctgtccttggtggtgtccggactggcctgcggtcctggcagaggctacggcagaagaagacatccgaagaagctgacacctctcgcctacaagcagttcatacctaatgtcgcggagaagaccttaggggccagcggcagatacgagggcaagataacgcgcaattcggagagatttaaagaacttactccaaattacaatcccgacattatctttaaggatgaggagaacacgggagcggacaggctcatgacacagagatgcaaagacaagctgaactcgctggccatctctgtaatgaaccactggccaggggttaagctgcgtgtgacagagggctgggatgaggacggtcaccattttgaagaatcactccactacgagggaagagctgttgatattaccacctctgaccgagacaagagcaaatacgggacactgtctcgcctagctgtggaggctggatttgactgggtctattacgagtccaaagcccacattcattgctctgtcaaagcagaaaattcggttgctgcgaaatctgggggctgtttcccaggttcggctctggtctcgctccaggacggaggacagaaggccgtgaaggacctgaaccccggagacaaggtgctggcggcagacagcgcgggaaacctggtgttcagcgacttcatcatgttcacagaccgagactccacgacgcgacgtgtgttttacgtcatagaaacgcaagaacccgttgaaaagatcaccctcaccgccgctcacctcctttttgtcctcgacaactcaacggaagatctccacaccatgaccgccgcgtatgccagcagtgtcagagccggacaaaaggtgatggttgttgatgatagcggtcagcttaaatctgtcatcgtgcagcggatatacacggaggagcagcggggctcgttcgcaccagtgactgcacatgggaccattgtggtcgacagaatactggcgtcctgttacgccgtaatagaggaccaggggcttgcgcatttggccttcgcgcccgccaggctctattattacgtgtcatcattcctgtcccccaaaactccagcagtcggtccaatgcgactttacaacaggagggggtccactggtactccaggctcctgtcatcaaatgggaacgtggcttttggacagcaacatgcttcatcctttggggatgtcagtaaactcaagctg

SEQ ID NO: 6 - DNA, 1425 bp, Homo sapiens
  CDS (1)...(1425)
  misc_feature 1387, 1388, 1389: n = A,T,C or G
atgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagcnnnagccggggggccgggggaggggcgcgggagggggcc

SEQ ID NO: 7 - DNA, 1622 bp, Homo sapiens
  CDS (51)...(1283)
catcagcccaccaggagacctcgcccgccgctcccccgggctccccggccatgtctcccgcccggctccggccccgactgcacttctgcctggtcctgttgctgctgctggtggtgcccgcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagccccacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccgcgtcgtggtgtagtcatagagctgcaagctgagctggcgaggggatggttgttgacccctctctcctagagaccttgaggctggcacggcgactcccaactcagcctgctctcactacgagttttcatactctgcctcccccattgggagggcccattccc

SEQ ID NO: 8 - DNA, 1190 bp, Homo sapiens
  CDS (1)...(1190)
atggctctcctgaccaatctactgcccttgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgcaaggagagggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggctg

SEQ ID NO: 9 - DNA, 1251 bp, Brachydanio rerio
  CDS (1)...(1248)
atggacgtaaggctgcatctgaagcaatttgctttactgtgttttatcagcttgcttctgacgccttgtggattagcctgtggtcctggtagaggttatggaaaacgaagacacccaaagaaattaaccccgttggcttacaagcaattcatccccaacgttgctgagaaaacgcttggagccagcggcaaatacgaaggcaaaatcacaaggaattcagagagatttaaagagctgattccgaattataatcccgatatcatctttaaggacgaggaaaacacaaacgctgacaggctgatgaccaagcgctgtaaggacaagttaaattcgttggccatatccgtcatgaaccactggcccggcgtgaaactgcgcgtcactgaaggctgggatgaggatggtcaccatttagaagaatctttgcactatgagggacgggcagtggacatcactacctcagacagggataaaagcaagtatgggatgctatccaggcttgcagtggaggcaggattcgactgggtctattatgaatctaaagcccacatacactgctctgtcaaagcagaaaattcagtggctgctaaatcaggaggatgttttcctgggtctgggacggtgacacttggtgatgggacgaggaaacccatcaaagatcttaaagtgggcgaccgggttttggctgcagacgagaagggaaatgtcttaataagcgactttattatgtttatagaccacgatccgacaacgagaaggcaattcatcgtcatcgagacgtcagaacctttcaccaagctcaccctcactgccgcgcacctagttttcgttggaaactcttcagcagcttcgggtataacagcaacatttgccagcaacgtgaagcctggagatacagttttagtgtgggaagacacatgcgagagcctcaagagcgttacagtgaaaaggatttacactgaggagcacgagggctcttttgcgccagtcaccgcgcacggaaccataatagtggatcaggtgttggcatcgtgctacgcggtcattgagaaccacaaatgggcacattgggcttttgcgccggtcaggttgtgtcacaagctgatgacgtggctttttccggctcgtgaatcaaacgtcaattttcaggaggatggtatccactggtactcaaatatgctgtttcacatcggctcttggctgctggacagagactctttccatccactcgggattttacacttaagttga

SEQ ID NO: 10 - protein, 425 aa, Gallus gallus
MVEMLLLTRILLVGFICALLVSSGLTCGPGRGIGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEHGGTKLVKDLSPGDRVLAADADGRLLYSDFLTFLDRMDSSRKLFYVIETRQPRARLLLTAAHLLFVAPQHNQSEATGSTSGQALFASNVKPGQRVYVLGEGGQQLLPASVHSVSLREEASGAYAPLTAQGTILINRVLASCYAVIEEHSWAHWAFAPFRLAQGLLAALCPDGAIPTAATTTTGIHWYSRLLYRIGSWVLDGDALHPLGMVAPAS

SEQ ID NO: 11 - protein, 396 aa, Mus musculus [sequence omitted]

SEQ ID NO: 12 - protein, 411 aa, Mus musculus
MSPAWLRPRLRFCLFLLLLLLVPAARGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGRDEDGHHSEESLHYEGRAVDITTSDRDRNKYGLLARLAVEAGFDWVYYESKAHVHCSVKSEHSAAAKTGGCFPAGAQVRLENGERVALSAVKPGDRVLAMGEDGTPTFSDVLIFLDREPNRLRAFQVIETQDPPRRLALTPAHLLFIADNHTEPAAHFRATFASHVQPGQYVLVSGVPGLQPARVAAVSTHVALGSYAPLTRHGTLVVEDVVASCFAAVADHHLAQLAFWPLRLFPSLAWGSWTPSEGVHSYPQMLYRLGRLLLEESTFHPLGMSGAGS

SEQ ID NO: 13 - protein, 437 aa, Mus musculus
MLLLLARCFLVILASSLLVCPGLACGPGRGFGKRRHPKKLTPLAYKQFIPNVAEKTLGASGRYEGKITRNSERFKELTPNYNPDIIFKDEENTGADRLMTQRCKDKLNALAISVMNQWPGVRLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLRPGDRVLAADDQGRLLYSDFLTFLDRDEGAKKVFYVIETLEPRERLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS

SEQ ID NO: 14 - protein, 418 aa, Brachydanio rerio
MRLLTRVLLVSLLTLSLVVSGLACGPGRGYGRRRHPKKLTPLAYKQFIPNVAEKTLGASGRYEGKITRNSERFKELTPNYNPDIIFKDEENTGADRLMTQRCKDKLNSLAISVMNHWPGVKLRVTEGWDEDGHHFEESLHYEGRAVDITTSDRDKSKYGTLSRLAVEAGFDWVYYESKAHIHCSVKAENSVAAKSGGCFPGSALVSLQDGGQKAVKDLNPGDKVLAADSAGNLVFSDFIMFTDRDSTTRRVFYVIETQEPVEKITLTAAHLLFVLDNSTEDLHTMTAAYASSVRAGQKVMVVDDSGQLKSVIVQRIYTEEQRGSFAPVTAHGTIVVDRILASCYAVIEDQGLAHLAFAPARLYYYVSSFLSPKTPAVGPMRLYNRRGSTGTPGSCHQMGTWLLDSNMLHPLGMSVNSS

SEQ ID NO: 15 - protein, 475 aa, Homo sapiens
  VARIANT 463: Xaa = Any Amino Acid
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSSXSRGAGGGAREGA

SEQ ID NO: 16 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 17 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 18 - protein, 416 aa, Brachydanio rerio
MDVRLHLKQFALLCFISLLLTPCGLACGPGRGYGKRRHPKKLTPLAYKQFIPNVAEKTLGASGKYEGKITRNSERFKELIPNYNPDIIFKDEENTNADRLMTKRCKDKLNSLAISVMNHWPGVKLRVTEGWDEDGHHLEESLHYEGRAVDITTSDRDKSKYGMLSRLAVEAGFDWVYYESKAHIHCSVKAENSVAAKSGGCFPGSGTVTLGDGTRKPIKDLKVGDRVLAADEKGNVLISDFIMFIDHDPTTRRQFIVIETSEPFTKLTLTAAHLVFVGNSSAASGITATFASNVKPGDTVLVWEDTCESLKSVTVKRIYTEEHEGSFAPVTAHGTIIVDQVLASCYAVIENHKWAHWAFAPVRLCHKLMTWLFPARESNVNFQEDGIHWYSNMLFHIGSWLLDRDSFHPLGILHLS

SEQ ID NO: 19 - DNA, 1416 bp, Drosophila melanogaster
  CDS (1)...(1413)
atggataaccacagctcagtgccttgggccagtgccgccagtgtcacctgtctctccctgggatgccaaatgccacagttccagttccagttccagctccaaatccgcagcgagctccatctccgcaagcccgcaagaagaacgcaaacgatgcgccacattgcgcatacgcagcgttgcctcagcaggctgacctctctggtggccctgctgctgatcgtcttgccgatggtctttagcccggctcacagctgcggtcctggccgaggattgggtcgtcatagggcgcgcaacctgtatccgctggtcctcaagcagacaattcccaatctatccgagtacacgaacagcgcctccggacctctggagggtgtgatccgtcgggattcgcccaaattcaaggacctcgtgcccaactacaacagggacatccttttccgtgacgaggaaggcaccggagcggatggcttgatgagcaagcgctgcaaggagaagctaaacgtgctggcctactcggtgatgaacgaatggcccggcatccggctgctggtcaccgagagctgggacgaggactaccatcacggccaggagtcgctccactacgagggccgagcggtgaccattgccacctccgatcgcgaccagtccaaatacggcatgctcgctcgcctggccgtcgaggctggattcgattgggtctcctacgtcagcaggcgccacatctactgctccgtcaagtcagattcgtcgatcagttcccacgtgcacggctgcttcacgccggagagcacagcgctgctggagagtggagtccggaagccgctcggcgagctctctatcggagatcgtgttttgagcatgaccgccaacggacaggccgtctacagcgaagtgatcctcttcatggaccgcaacctcgagcagatgcaaaactttgtgcagctgcacacggacggtggagcagtgctcacggtgacgccggctcacctggttagcgtttggcagccggagagccagaagctcacgtttgtgtttgcgcatcgcatcgaggagaagaaccaggtgctcgtacgggatgtggagacgggcgagctgaggccccagcgagtggtcaagttgggcagtgtgcgcagtaagggcgtggtcgcgccgctgacccgcgagggcaccattgtggtcaactcggtggccgccagttgctatgcggtgatcaacagtcagtcgctggcccactggggactggctcccatgcgcctgctgtccacgctggaggcgtggctgcccgccaaggagcagttgcacagttcgccgaaggtggtgagctcggcgcagcagcagaatggcatccattggtatgccaatgcgctctacaaggtcaaggactacgtgctgccgcagagctggcgccacgattga

SEQ ID NO: 20 - protein, 471 aa, Drosophila melanogaster
MDNHSSVPWASAASVTCLSLGCQMPQFQFQFQLQIRSELHLRKPARRTQTMRHIAHTQRCLSRLTSLVALLLIVLPMVFSPAHSCGPGRGLGRHRARNLYPLVLKQTIPNLSEYTNSASGPLEGVIRRDSPKFKDLVPNYNRDILFRDEEGTGADGLMSKRCKEKLNVLAYSVMNEWPGIRLLVTESWDEDYHHGQESLHYEGRAVTIATSDRDQSKYGMLARLAVEAGFDWVSYVSRRHIYCSVKSDSSISSHVHGCFTPESTALLESGVRKPLGELSIGDRVLSMTANGQAVYSEVILFMDRNLEQMQNFVQLHTDGGAVLTVTPAHLVSVWQPESQKLTFVFAHRIEEKNQVLVRDVETGELRPQRVVKLGSVRSKGVVAPLTREGTIVVNSVAASCYAVINSQSLAHWGLAPMRLLSTLEAWLPAKEQLHSSPKVVSSAQQQNGIHWYANALYKVKDYVLPQSWRHD

SEQ ID NO: 21 - protein, 221 aa, Artificial Sequence
  Degenerate Shh polypeptide general formula
CGPGRGXGXRRHPKKLTPLAYKQFIPNVAEKTLGASGRYEGKIXRNSERFKELTPNYNPDIIFKDEENTGADRLMTQRCKDKLNXLAISVMNXWPGVXLRVTEGWDEDGHHXEESLHYEGRAVDITTSDRDXSKYGXLXRLAVEAGFDWVYYESKAHIHCSVKAENSVAAKSGGCFPGSAXVXLXXGGXKXVKDLXPGDXVLAADXXGXLXXSDFXXFXDR

SEQ ID NO: 22 - protein, 167 aa, Artificial Sequence
  Degenerate hedgehog polypeptide general formula
CGPGRGXXXRRXXXPKXLXPLXYKQFXPXXXEXTLGASGXXEGXXXRXSERFXXLTPNYNPDIIFKDEENXGADRLMTXRCKXXXNXLAISVMNXWPGVXLRVTEGXDEDGHHXXXSLHYEGRAXDITTSDRDXXKYGXLXRLAVEAGFDWVYYESXXHXHXSVKXX

SEQ ID NO: 23 - DNA, 3900 bp, Drosophila melanogaster
  CDS (1)...(3897)
atggaccgcgacagcctcccacgcgttccggacacacacggcgatgtggtcgatgagaaattattctcggatctttacatacgcaccagctgggtggacgcccaagtggcgctcgatcagatagataagggcaaagcgcgtggcagccgcacggcgatctatctgcgatcagtattccagtcccacctcgaaaccctcggcagctccgtgcaaaagcacgcgggcaaggtgctattcgtggctatcctggtgctgagcaccttctgcgtcggcctgaagagcgcccagatccactccaaggtgcaccagctgtggatccaggagggcggcgggctggaggcggaactggcctacacacagaagacgatcggcgaggacgagtcggccacgcatcagctgctcattcagacgacccacgacccgaacgcctccgtcctgcatccgcaggcgctgcttgcccacctggaggtcctggtcaaggccaccgccgtcaaggtgcacctctacgacaccgaatgggggctgcgcgacatgtgcaacatgccgagcacgccctccttcgagggcatctactacatcgagcagatcctgcgccacctcattccgtgctcgatcatcacgccgctggactgtttctgggagggaagccagctgttgggtccggaatcagcggtcgttataccaggcctcaaccaacgactcctgtggaccacactgaatcccgcctctgtgatgcagtatatgaagcagaagatgtccgaggaaaagatcagcttcgacttcgagaccgtggagcagtacatgaagcgtgcggccattgcgagtggctacatggagaagccctgcctgaacccactgaatcccaattgcccggacacggcaccgaacaagaacagcacccagccgccggatgtgggagccatcctgtccggaggctgctacggttatgccgcgaagcacatgcactggccggaggagctgattgtgggcggagcgaagaggaaccgcagcggacacttgaggaaggcccaggccctgcagtcggtggtgcagctgatgaccgagaaggaaatgtacgaccagtggcaggacaactacaaggtgcaccatcttggatggacgcaggagaaggcagcggaggttttgaacgcctggcagcgcaacttttcgcgggaggtggaacagctgctacgtaaacagtcgagaattgccaccaactacgatatctacgtgttcagctcggctgcactggatgacatcctggccaagttctcccatcccagcgccttgtccattgtcatcggcgtggccgtcaccgttttgtatgccttctgcacgctcctccgctggagggaccccgtccgtggacagagcagtgtcggcgtggccggagttctgctcatgtgctttagtaccgccgccggattgggattgtcagccctgctcggtatcgttttcaatgccgccagcacccaggtggttccgtttttggcccttggtctgggcgtcgatcacatcttcatgctgaccgctgcctatgcggagagcaatcggcgggagcagaccaagctgattctcaagaaagtgggaccgagcatcctgttcagtgcctgcagcaccgcaggatccttctttgcggccgcctttattccggtgccggctttgaaggtattctgtctgcaggctgccatcgtaatgtgctccaatttggcagcggctctattggtttttccggccatgatttcgttggatctacggagacgtaccgccggcagggcggacatcttctgctgctgttttccggtgtggaaggaacagccgaaggtggcaccaccggtgctgccgctgaacaacaacaacgggcgcggggcccggcatccgaagagctgcaacaacaacagggtggcgctgcccgcccagaatcctctgctggaacagagggcagacatccctgggagcagtcactcactggcgtccttctctctggcaacattcgcctttcagcactacactcccttcctcatgcgcagctgggtgaagttcctgaccgttatgggtttcctggcggccctcatatccagcttgtatgcctccacgcgccttcaggatggcctggacattattgatctggtgcccaaggacagcaacgagcacaagttcctggatgctcaaactcggctctttggcttctacagcatgtatgcggttacccagggcaactttgaatatcccacccagcagcagttgctcagggactaccatgattcctttgtgcgggtgccacatgtgatcaagaatgataatggtggactgccggacttctggctgctgctcttcagcgagtggctgggtaatctgcaaaagatattcgacgaggaataccgcgacggacggctgaccaaggagtgctggttcccaaacgccagcagcgatgccatcctggcctacaagctaatcgtgcaaaccggccatgtggacaaccccgtggacaaggaactggtgctcaccaatcgcctggtcaacagcgatggcatcatcaaccaacgcgccttctacaactatctgtcggcatgggccaccaacgcgtcttcgcctacggagcttctcagggcaaattgtatccggaaccgcgccaacggagcttctcagggcaaattgtatccggaaccgcgccagtattttcaccaacccaacgagtacgatcttaagatacccaagagtctgccattggtctacgctcagatgcccttttacctccacggactaacagatacctcgcagatcaagaccctgataggtcatattcgcgacctgagcgtcaagtacgagggcttcggcctgcccaactatccatcgggcattcccttcatcttctgggagcagtacatgaccctgcgctcctcactggccatgatcctggcctgcgtgctactcgccgccctggtgctggtctccctgctcctgctctccgtttgggccgccgttctcgtgatcctcagcgttctggcctcgctggcccagatctttggggccatgactctgctgggcatcaaactctcggccattccggcagtcatactcatcctcagcgtgggcatgatgctgtgcttcaatgtgctgatatcactgggcttcatgacatccgttggcaaccgacagcgccgcgtccagctgagcatgcagatgtccctgggaccacttgtccacggcatgctgacctccggagtggccgtgttcatgctctccacgtcgccctttgagtttgtgatccggcacttctgctggcttctgctggtggtcttatgcgttggcgcctgcaacagccttttggtgttccccatcctactgagcatggtgggaccggaggcggagctggtgccgctggagcatccagaccgcatatccacgccctctccgctgcccgtgcgcagcagcaagagatcgggcaaatcctatgtggtgcagggatcgcgatcctcgcgaggcagctgccagaagtcgcatcaccaccaccacaaagaccttaatgatccatcgctgacgacgatcaccgaggagccgcagtcgtggaagtccagcaactcgtccatccagatgcccaatgattggacctaccagccgcgggaacagcgacccgcctcctacgcggccccgccccccgcctatcacaaggccgccgcccagcagcaccaccagcatcagggcccgcccacaacgcccccgccgcccttcccgacggcctatccgccggagctgcagagcatcgtggtgcagccggaggtgacggtggagacgacgcactcggacagcaacaccaccaaggtgacggccacggccaacatcaaggtggagctggccatgcccggcagggcggtgcgcagctataactttacgagttag